SEQUENCE LISTING (58 sequences)

SEQ ID NO: 1 - protein, 5 aa, synthetic construct
DYNMD

SEQ ID NO: 2 - protein, 5 aa, synthetic construct
SYNMD

SEQ ID NO: 3 - protein, 17 aa, synthetic construct
DINPHSGGPIYNQKFTG

SEQ ID NO: 4 - protein, 17 aa, synthetic construct
DINPHNGGPIYNQKFTG

SEQ ID NO: 5 - protein, 17 aa, synthetic construct
DINPHGGGPIYNQKFTG

SEQ ID NO: 6 - protein, 17 aa, synthetic construct
DINPHQGGPIYNQKFTG

SEQ ID NO: 7 - protein, 10 aa, synthetic construct
GELGHWYFDV

SEQ ID NO: 8 - protein, 10 aa, synthetic construct
GELAHWYFDV

SEQ ID NO: 9 - protein, 15 aa, synthetic construct
RASKSVSTSGYSYIH

SEQ ID NO: 10 - protein, 7 aa, synthetic construct
LASNLES

SEQ ID NO: 11 - protein, 7 aa, synthetic construct
LASNLDS

SEQ ID NO: 12 - protein, 7 aa, synthetic construct
LASNLAS

SEQ ID NO: 13 - protein, 9 aa, synthetic construct
QHSRDLPPT

SEQ ID NO: 14 - protein, 119 aa, synthetic construct
EVQLVQSGAEVKKPGASVKISCKASGYTFTDYNMDWVKQAPGQRLEWIGDINPHSGGPIYNQKFTGRATLTVDKSASTAYMELSSLRSEDTAVYYCARGELGHWYFDVWGQGTTVTVSS

SEQ ID NO: 15 - protein, 119 aa, synthetic construct
EVQLVQSGAEVKKPGASVKISCKASGYTFTDYNMDWVKQAPGQRLEWIGDINPHNGGPIYNQKFTGRATLTVDKSASTAYMELSSLRSEDTAVYYCARGELGHWYFDVWGQGTTVTVSS

SEQ ID NO: 16 - protein, 119 aa, synthetic construct
EVQLVQSGAEVKKPGASVKISCKASGYTFTSYNMDWVKQAPGQRLEWIGDINPHNGGPIYNQKFTGRATLTVDKSASTAYMELSSLRSEDTAVYYCARGELGHWYFDVWGQGTTVTVSS

SEQ ID NO: 17 - protein, 119 aa, synthetic construct
EVQLVQSGAEVKKPGASVKISCKASGYTFTDYNMDWVKQAPGQRLEWIGDINPHGGGPIYNQKFTGRATLTVDKSASTAYMELSSLRSEDTAVYYCARGELGHWYFDVWGQGTTVTVSS

SEQ ID NO: 18 - protein, 119 aa, synthetic construct
EVQLVQSGAEVKKPGASVKISCKASGYTFTDYNMDWVKQAPGQRLEWIGDINPHQGGPIYNQKFTGRATLTVDKSASTAYMELSSLRSEDTAVYYCARGELGHWYFDVWGQGTTVTVSS

SEQ ID NO: 19 - protein, 111 aa, synthetic construct
DIVLTQSPASLALSPGERATLSCRASKSVSTSGYSYIHWYQQKPGQAPRLLIYLASNLESGVPARFSGSGSGTDFTLTISSLEEEDFATYYCQHSRDLPPTFGQGTKLEIK

SEQ ID NO: 20 - protein, 111 aa, synthetic construct
DIVLTQSPASLALSPGERATLSCRASKSVSTSGYSYIHWYQQKPGQAPRLLIYLASNLDSGVPARFSGSGSGTDFTLTISSLEEEDFATYYCQHSRDLPPTFGQGTKLEIK

SEQ ID NO: 21 - protein, 111 aa, synthetic construct
DIVLTQSPASLALSPGERATLSCRASKSVSTSGYSYIHWYQQKPGQAPRLLIYLASNLASGVPARFSGSGSGTDFTLTISSLEEEDFATYYCQHSRDLPPTFGQGTKLEIK

SEQ ID NO: 22 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 24 - protein, 107 aa, synthetic construct [sequence omitted]

SEQ ID NO: 25 - protein, 107 aa, synthetic construct [sequence omitted]

SEQ ID NO: 26 - protein, 448 aa, synthetic construct
EVQLVQSGAEVKKPGASVKISCKASGYTFTDYNMDWVKQAPGQRLEWIGDINPHSGGPIYNQKFTGRATLTVDKSASTAYMELSSLRSEDTAVYYCARGELGHWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 27 - protein, 448 aa, synthetic construct
EVQLVQSGAEVKKPGASVKISCKASGYTFTDYNMDWVKQAPGQRLEWIGDINPHNGGPIYNQKFTGRATLTVDKSASTAYMELSSLRSEDTAVYYCARGELGHWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 28 - protein, 448 aa, synthetic construct
EVQLVQSGAEVKKPGASVKISCKASGYTFTSYNMDWVKQAPGQRLEWIGDINPHNGGPIYNQKFTGRATLTVDKSASTAYMELSSLRSEDTAVYYCARGELGHWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 29 - protein, 448 aa, synthetic construct
EVQLVQSGAEVKKPGASVKISCKASGYTFTDYNMDWVKQAPGQRLEWIGDINPHGGGPIYNQKFTGRATLTVDKSASTAYMELSSLRSEDTAVYYCARGELGHWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 30 - protein, 448 aa, synthetic construct
EVQLVQSGAEVKKPGASVKISCKASGYTFTDYNMDWVKQAPGQRLEWIGDINPHQGGPIYNQKFTGRATLTVDKSASTAYMELSSLRSEDTAVYYCARGELGHWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 31 - protein, 218 aa, synthetic construct
DIVLTQSPASLALSPGERATLSCRASKSVSTSGYSYIHWYQQKPGQAPRLLIYLASNLESGVPARFSGSGSGTDFTLTISSLEEEDFATYYCQHSRDLPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 32 - protein, 218 aa, synthetic construct
DIVLTQSPASLALSPGERATLSCRASKSVSTSGYSYIHWYQQKPGQAPRLLIYLASNLDSGVPARFSGSGSGTDFTLTISSLEEEDFATYYCQHSRDLPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - protein, 218 aa, synthetic construct
DIVLTQSPASLALSPGERATLSCRASKSVSTSGYSYIHWYQQKPGQAPRLLIYLASNLASGVPARFSGSGSGTDFTLTISSLEEEDFATYYCQHSRDLPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - DNA, 499 bp, synthetic construct
  source 1..499: mol_type = other DNA
gtttaaacggatctctagcgaattcgccgccaccatgggctggtcctgtatcatcctgttcctggtggccaccgctaccggcgtgcactccgacatcgtgctgacacagagccccgcaagcctggcactgtcccctggcgagagggccacactgagctgtagggcctccaagagcgtgagcacctccgggtactcctacatccactggtaccagcagaagcccgggcaggctcctagactgctgatctatctggcctccaatctggagagcggcgtgcctgcccgcttcagcggaagcggctccgggaccgatttcaccctgactatctccagcctggaggaggaggatttcgccacatactactgtcagcactccagggacctgcctccaacattcggccaggggaccaagctggagatcaagcgtactgtggctgcaccatctgtcttcatcttcccgccatctgatgagcaattgaaatctggaactgcctctgtt

SEQ ID NO: 35 - DNA, 506 bp, synthetic construct
  source 1..506: mol_type = other DNA
gtttaaacggatctctagcgaattcgccgccaccatgggctggtcctgtatcatcctgttcctggtggccaccgctaccggcgtgcactccgacatcgtgctgacacagagccccgcaagcctggcactgtcccctggcgagagggccacactgagctgtagggcctccaagagcgtgagcacctccgggtactcccaatctgtacatccactggtaccagcagaagcccgggcaggctcctagactgctgatctatctggcctccaatctggctagcggcgtgcctgcccgcttcagcggaagcggctccgggaccgatttcaccctgactatctccagcctggaggaggaggatttcgccacatactactgtcagcactccagggacctgcctccaacattcggccaggggaccaagctggagatcaagcgtactgtggctgcaccatctgtcttcatcttcccgccatctgatgagcaattgaaatctggaactgcctctgtt

SEQ ID NO: 36 - DNA, 499 bp, synthetic construct
  source 1..499: mol_type = other DNA
gtttaaacggatctctagcgaattcgccgccaccatgggctggtcctgtatcatcctgttcctggtggccaccgctaccggcgtgcactccgacatcgtgctgacacagagccccgcaagcctggcactgtcccctggcgagagggccacactgagctgtagggcctccaagagcgtgagcacctccgggtactcctacatccactggtaccagcagaagcccgggcaggctcctagactgctgatctatctggcctccaatctggatagcggcgtgcctgcccgcttcagcggaagcggctccgggaccgatttcaccctgactatctccagcctggaggaggaggatttcgccacatactactgtcagcactccagggacctgcctccaacattcggccaggggaccaagctggagatcaagcgtactgtggctgcaccatctgtcttcatcttcccgccatctgatgagcaattgaaatctggaactgcctctgtt

SEQ ID NO: 37 - DNA, 505 bp, synthetic construct
  source 1..505: mol_type = other DNA
gtttaaacggatctctagcgaattcgccgccaccatgggctggtcctgtatcatcctgttcctggtggccaccgctaccggcgtgcactccgaggtgcagctggtgcagagcggcgccgaggtgaagaagccaggcgccagcgtgaagatcagctgcaaggccagcggctacaccttcaccagctacaacatggactgggtgaagcaggccccaggccagcgcctggagtggatcggcgacatcaacccacacaacggcggcccaatctacaaccagaagttcaccggccgcgccaccctgaccgtggacaagagcgccagcaccgcctacatggagctgagcagcctgcgcagcgaggacaccgccgtgtactactgcgcccgcggcgagctgggccactggtacttcgacgtgtggggccagggcaccaccgtgaccgtgagcagcgcctccaccaagggcccatcggtcttcccgctagcaccctcctccaagagcacctct

SEQ ID NO: 38 - DNA, 505 bp, synthetic construct
  source 1..505: mol_type = other DNA
gtttaaacggatctctagcgaattcgccgccaccatgggctggtcctgtatcatcctgttcctggtggccaccgctaccggcgtgcactccgaggtgcagctggtgcagagcggcgccgaggtgaagaagccaggcgccagcgtgaagatcagctgcaaggccagcggctacaccttcaccgactacaacatggactgggtgaagcaggccccaggccagcgcctggagtggatcggcgacatcaacccacaccagggcggcccaatctacaaccagaagttcaccggccgcgccaccctgaccgtggacaagagcgccagcaccgcctacatggagctgagcagcctgcgcagcgaggacaccgccgtgtactactgcgcccgcggcgagctgggccactggtacttcgacgtgtggggccagggcaccaccgtgaccgtgagcagcgcctccaccaagggcccatcggtcttcccgctagcaccctcctccaagagcacctct

SEQ ID NO: 39 - DNA, 505 bp, synthetic construct
  source 1..505: mol_type = other DNA
gtttaaacggatctctagcgaattcgccgccaccatgggctggtcctgtatcatcctgttcctggtggccaccgctaccggcgtgcactccgaggtgcagctggtgcagagcggcgccgaggtgaagaagccaggcgccagcgtgaagatcagctgcaaggccagcggctacaccttcaccgactacaacatggactgggtgaagcaggccccaggccagcgcctggagtggatcggcgacatcaacccacacggcggcggcccaatctacaaccagaagttcaccggccgcgccaccctgaccgtggacaagagcgccagcaccgcctacatggagctgagcagcctgcgcagcgaggacaccgccgtgtactactgcgcccgcggcgagctgggccactggtacttcgacgtgtggggccagggcaccaccgtgaccgtgagcagcgcctccaccaagggcccatcggtcttcccgctagcaccctcctccaagagcacctct

SEQ ID NO: 40 - DNA, 505 bp, synthetic construct
  source 1..505: mol_type = other DNA
gtttaaacggatctctagcgaattcgccgccaccatgggctggtcctgtatcatcctgttcctggtggccaccgctaccggcgtgcactccgaggtgcagctggtgcagagcggcgccgaggtgaagaagccaggcgccagcgtgaagatcagctgcaaggccagcggctacaccttcaccgactacaacatggactgggtgaagcaggccccaggccagcgcctggagtggatcggcgacatcaacccacacagcggcggcccaatctacaaccagaagttcaccggccgcgccaccctgaccgtggacaagagcgccagcaccgcctacatggagctgagcagcctgcgcagcgaggacaccgccgtgtactactgcgcccgcggcgagctgggccactggtacttcgacgtgtggggccagggcaccaccgtgaccgtgagcagcgcctccaccaagggcccatcggtcttcccgctagcaccctcctccaagagcacctct

SEQ ID NO: 41 - DNA, 505 bp, synthetic construct
  source 1..505: mol_type = other DNA
gtttaaacggatctctagcgaattcgccgccaccatgggctggtcctgtatcatcctgttcctggtggccaccgctaccggcgtgcactccgaggtgcagctggtgcagagcggcgccgaggtgaagaagccaggcgccagcgtgaagatcagctgcaaggccagcggctacaccttcaccgactacaacatggactgggtgaagcaggccccaggccagcgcctggagtggatcggcgacatcaacccacacaacggcggcccaatctacaaccagaagttcaccggccgcgccaccctgaccgtggacaagagcgccagcaccgcctacatggagctgagcagcctgcgcagcgaggacaccgccgtgtactactgcgcccgcggcgagctgggccactggtacttcgacgtgtggggccagggcaccaccgtgaccgtgagcagcgcctccaccaagggcccatcggtcttcccgctagcaccctcctccaagagcacctct

SEQ ID NO: 42 - protein, 330 aa, synthetic construct; identical to SEQ ID NO: 22

SEQ ID NO: 43 - protein, 107 aa, synthetic construct [sequence omitted]

SEQ ID NO: 44 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 45 - protein, 107 aa, synthetic construct [sequence omitted]

SEQ ID NO: 46 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 107 aa, synthetic construct [sequence omitted]

SEQ ID NO: 48 - protein, 199 aa, Homo sapiens
MNCVCRLVLVVLSLWPDTAVAPGPPPGPPRVSPDPRAELDSTVLLTRSLLADTRQLAAQLRDKFPADGDHNLDSLPTLAMSAGALGALQLPGVLTRLRADLLSYLRHVQWLRRAGGSSLKTLEPELGTLQARLDRLLRRLQLLMSRLALPQPPPDPPAPPLAPPSSAWGGIRAAHAILGGLHLTLDWAVRGLLLLKTRL

SEQ ID NO: 49 - protein, 188 aa, Homo sapiens
PGPPPGPPRVSPDPRAELDSTVLLTRSLLADTRQLAAQLRDKFPADGDHNLDSLPTLAMSAGALGALQLPGVLTRLRADLLSYLRHVQWLRRAGGSSLKTLEPELGTLQARLDRLLRRLQLLMSRLALPQPPPDPPAPPLAPPSSAWGGIRAAHAILGGLHLTLDWAVRGLLLLKTRLGGSGHHHHHH

SEQ ID NO: 50 - protein, 5 aa, synthetic construct
  VARIANT 1: note = D or S
XYNMD

SEQ ID NO: 51 - protein, 17 aa, synthetic construct
  VARIANT 6: note = S, N, G, or Q
DINPHXGGPIYNQKFTG

SEQ ID NO: 52 - protein, 7 aa, synthetic construct
  VARIANT 6: note = E, D, or A
LASNLXS

SEQ ID NO: 53 - protein, 455 aa, synthetic construct
QVQLVESGGGLVKPGGSLRLSCAASGFTFSDYAMSWIRQAPGKGLEWVSSINIGATYIYYADSVKGRFTISRDNAKNSLYLQMNSLRAEDTAVYYCARPGSPYEYDKAYYSMAYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 214 aa, synthetic construct
DIQMTQSPSSLSASVGDRVTITCRASQDIKNYLNWYQQKPGKAPKLLIYYSSTLLSGVPSRFSGSGSGTDFTLTISSLQPEDFATYYCQQSITLPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 55 - protein, 446 aa, synthetic construct
QVTLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGAGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 56 - protein, 213 aa, synthetic construct
DIQMTQSPSTLSASVGDRVTITCKCQLSVGYMHWYQQKPGKAPKLLIYDTSKLASGVPSRFSGSGSGTEFTLTISSLQPDDFATYYCFQGSGYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 57 - protein, 449 aa, synthetic construct
EVQLVQSGAEVKKPGASVKISCKASGYTFTDYNMDWVKQAPGQRLEWIGDINPHSGGPIYNQKFTGRATLTVDKSASTAYMELSSLRSEDTAVYYCARGELGHWYFDVWGQGTTVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 58 - protein, 218 aa, synthetic construct
DIVLTQSPASLALSPGERATLSCRASKSVSTSGYSYIHWYQQKPGQAPRLLIYLASNLESGVPARFSGSGSGTDFTLTISSLEEEDFATYYCQHSRDLPPTFGQGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

(19) United States
(12) Patent Application Publication
(10) Pub. No.: US 2026/0242459 A1
(43) Pub. Date: Aug. 20, 2026

(54) ANTI-INTERLEUKIN-11 ANTIBODIES AND METHODS OF USE THEREOF

(71) Applicant: Boehringer Ingelheim International GmbH, Ingelheim am Rhein (DE)

(72) Inventors: Maureen Crames, Ridgefield, CT (US); Pankaj Gupta, Ridgefield, CT (US); Julie Jai Li Hawkins, Ridgefield, CT (US); Siqi Lin, Ridgefield, CT (US); Michael S. Marlow, Ridgefield, CT (US)

(73) Assignee: Boehringer Ingelheim International GmbH, Ingelheim am Rhein (DE)

(21) Appl. No.: 19/542,772

(22) Filed: Feb. 18, 2026

Related U.S. Application Data
(60) Provisional application No. 63/760,414, filed on Feb. 19, 2025.

(51) Int. Cl. C07K 16/24; A61K 39/00; A61P 1/16; C12N 15/63

(52) U.S. Cl. CPC C07K 16/244; A61P 1/16; C12N 15/63; A61K 2039/505; C07K 2317/14; C07K 2317/24; C07K 2317/565; C07K 2317/92

(57) ABSTRACT

The present disclosure relates to anti-interleukin-11 (IL-11) antibodies and antigen-binding fragments thereof including their use in the treatment or prevention of an IL-11 (interleukin 11) pathway disease or disorder.

### RELATED APPLICATIONS

This application claims the benefit of priority to U.S. Provisional Application No. 63/760,414, filed Feb. 19, 2025, which is hereby incorporated by reference in its entirety.

### SEQUENCE LISTING

The instant application contains a Sequence Listing which has been submitted electronically in XML format and is hereby incorporated by reference in its entirety. Said XML copy, created on Feb. 9, 2026, is named 09-0760-US-3_SL.xml and is 64,283 bytes in size.

### FIELD

The present disclosure generally relates to anti-interleukin-11 (IL-11) antibodies including their use for the treatment of a fibrotic disease or disorder such as idiopathic pulmonary fibrosis (IPF), progressive pulmonary fibrosis (PPF), or a liver disease or disorder such as metabolic dysfunction-associated steatohepatitis (MASH).

### BACKGROUND

IL-11 is a secreted cytokine and a member of the IL-6 cytokine family which also includes IL-27, IL-31, leukemia inhibitory factor (LIF), oncostatin M (OSM) and ciliary neurotrophic factor (CNTF) among others. IL-6 family cytokines induce signal transduction via a common signal-transducing receptor β-subunit, gp130, and a specific receptor α-subunit. In the case of IL-11, binding of this cytokine to its specific receptor α-subunit, IL-11Rα, induces gp130 homodimerization. Dimerization of gp130 activates the JAK/STAT signaling pathway and leads to the activation of signal transducer and activator of transcription (STAT) 3 (STAT3) and to a lesser extent, STAT1. IL-11 upregulation and subsequent activity via interaction with IL-11Rα and heterogenic complex gp130 has been identified as a pathologic axis in several diseases, including fibrosis of the lungs, heart, liver and skin. Excessive fibrosis is common in many disease conditions and is important in disease pathogenesis. Despite the large impact on human health, therapeutic and diagnostic approaches to fibrosis remain an unmet medical need.

### SUMMARY

The disclosure addresses the above need by providing therapeutic agents for modulating (e.g., reducing or neutralizing) IL-11 binding and/or activity. The present disclosure provides methods of modulating IL-11 binding and/or activity, and methods of treating IL-11-associated diseases or conditions using an antibody or antigen-binding fragment thereof as described herein.

The present disclosure provides methods of using anti-IL-11 antibodies or antigen-binding fragments thereof that bind IL-11 (e.g., specifically bind IL-11 such as human IL-11), or compositions comprising anti-IL-11 antibodies or antigen-binding fragments thereof, for use in the treatment and/or prevention of diseases or disorders including, for example, inflammatory disease, autoimmune disease, respiratory disease, infectious disease, pulmonary fibrosis (e.g., IPF or PPF), or liver disease (e.g., MASH).

In some aspects, the disclosure provides an anti-IL-11 antibody or an antigen-binding fragment thereof comprising:
a heavy chain variable region (VH) comprising the amino acid sequence of SEQ ID NO: 1 or SEQ ID NO: 2 (H-CDR1), the amino acid sequence of SEQ ID NO: 3, SEQ ID NO: 4, SEQ ID NO: 5, or SEQ ID NO: 6 (H-CDR2), and the amino acid sequence of SEQ ID NO: 7 (H-CDR3); and
a light chain variable region (VL) comprising the amino acid sequence of SEQ ID NO: 9 (L-CDR1), the amino acid sequence of SEQ ID NO: 10, SEQ ID NO: 11, or SEQ ID NO: 12 (L-CDR2), and the amino acid sequence of SEQ ID NO: 13 (L-CDR3).

In some aspects, the disclosure provides an anti-IL-11 antibody or an antigen-binding fragment thereof comprising:
a heavy chain variable region (VH) comprising the amino acid sequence of SEQ ID NO: 1 (H-CDR1), the amino acid sequence of SEQ ID NO: 3 (H-CDR2), and the amino acid sequence of SEQ ID NO: 7 (H-CDR3); and
a light chain variable region (VL) comprising the amino acid sequence of SEQ ID NO: 9 (L-CDR1), the amino acid sequence of SEQ ID NO: 10 (L-CDR2), and the amino acid sequence of SEQ ID NO: 13 (L-CDR3).

In some embodiments, the anti-IL-11 antibody or antigen-binding fragment thereof comprises a VH that has about 80%, about 85%, about 90%, about 95%, about 96%, about 97%, about 98%, about 99% or about 100% sequence identity to the amino acid sequence of SEQ ID NO: 14, 15, 16, 17, or 18.

In some embodiments, the anti-IL-11 antibody or antigen-binding fragment thereof comprises a VH having the amino acid sequence of SEQ ID NO: 14, 15, 16, 17, or 18.

In some embodiments, the anti-IL-11 antibody or antigen-binding fragment thereof comprises a VL that has about 80%, about 85%, about 90%, about 95%, about 96%, about 97%, about 98%, about 99% or about 100% sequence identity to the amino acid sequence of SEQ ID NO: 19, 20, or 21.

In some embodiments, the anti-IL-11 antibody or antigen-binding fragment thereof comprises a VL having the amino acid sequence of SEQ ID NO: 19, 20, or 21.

In some embodiments, the anti-IL-11 antibody or antigen-binding fragment thereof comprises:
a VH that has about 80%, about 85%, about 90%, about 95%, about 96%, about 97%, about 98%, about 99% or about 100% sequence identity to the amino acid sequence of SEQ ID NOs: 14, 15, 16, 17, or 18; and
a VL that has about 80%, about 85%, about 90%, about 95%, about 96%, about 97%, about 98%, about 99% or about 100% sequence identity to SEQ ID NOs: 19, 20, or 21.

In some embodiments, the anti-IL-11 antibody or antigen-binding fragment thereof comprises a VH comprising the amino acid sequence of SEQ ID NOs: 14, 15, 16, 17, or 18; and a VL comprising the amino acid sequence of SEQ ID NOs: 19, 20, or 21.

In some aspects, the disclosure provides an anti-IL-11 antibody or antigen-binding fragment thereof comprising:
a VH that has about 80%, about 85%, about 90%, about 95%, about 96%, about 97%, about 98%, about 99% or about 100% sequence identity to the amino acid sequence of SEQ ID NOs: 14, 15, 16, 17, or 18; and
a VL that has about 80%, about 85%, about 90%, about 95%, about 96%, about 97%, about 98%, about 99% or about 100% sequence identity to SEQ ID NOs: 19, 20, or 21;
wherein the VH comprises the amino acid sequence of SEQ ID NO: 1 or SEQ ID NO: 2 (H-CDR1), the amino acid sequence of SEQ ID NO: 3, SEQ ID NO: 4, SEQ ID NO: 5, or SEQ ID NO: 6 (H-CDR2), and the amino acid sequence of SEQ ID NO: 7 (H-CDR3); and
wherein the VL comprises the amino acid sequence of SEQ ID NO: 9 (L-CDR1), the amino acid sequence of SEQ ID NO: 10, SEQ ID NO: 11, or SEQ ID NO: 12 (L-CDR2), and the amino acid sequence of SEQ ID NO: 13 (L-CDR3).

In some embodiments, the anti-IL-11 antibody or antigen-binding fragment thereof comprises:
a) a VH comprising the amino acid sequence of SEQ ID NO: 14, and a VL comprising the amino acid sequence of SEQ ID NO: 19;
b) a VH comprising the amino acid sequence of SEQ ID NO: 15, and a VL comprising the amino acid sequence of SEQ ID NO: 19;
c) a VH comprising the amino acid sequence of SEQ ID NO: 15, and a VL comprising the amino acid sequence of SEQ ID NO: 21;
d) a VH comprising the amino acid sequence of SEQ ID NO: 16, and a VL comprising the amino acid sequence of SEQ ID NO: 20;
e) a VH comprising the amino acid sequence of SEQ ID NO: 16, and a VL comprising the amino acid sequence of SEQ ID NO: 19;
f) a VH comprising the amino acid sequence of SEQ ID NO: 16, and a VL comprising the amino acid sequence of SEQ ID NO: 21;
g) a VH comprising the amino acid sequence of SEQ ID NO: 17, and a VL comprising the amino acid sequence of SEQ ID NO: 20;
h) a VH comprising the amino acid sequence of SEQ ID NO: 17, and a VL comprising the amino acid sequence of SEQ ID NO: 19;
i) a VH comprising the amino acid sequence of SEQ ID NO: 17, and a VL comprising the amino acid sequence of SEQ ID NO: 21;
j) a VH comprising the amino acid sequence of SEQ ID NO: 18, and a VL comprising the amino acid sequence of SEQ ID NO: 20;
k) a VH comprising the amino acid sequence of SEQ ID NO: 18, and a VL comprising the amino acid sequence of SEQ ID NO: 19;
l) a VH comprising the amino acid sequence of SEQ ID NO: 18, and a VL comprising the amino acid sequence of SEQ ID NO: 21;
m) a VH comprising the amino acid sequence of SEQ ID NO: 14, and a VL comprising the amino acid sequence of SEQ ID NO: 20; or
n) a VH comprising the amino acid sequence of SEQ ID NO: 14, and a VL comprising the amino acid sequence of SEQ ID NO: 21.

In some embodiments, the anti-IL-11 antibody or antigen-binding fragment thereof comprises an IgG1 constant region. In some embodiments, the IgG1 constant region is a human IgG1 constant region.

In some embodiments, the anti-IL-11 antibody or antigen-binding fragment thereof comprises a heavy chain constant region (CH) that has about 80%, about 85%, about 90%, about 95%, about 96%, about 97%, about 98%, about 99% or about 100% sequence identity to the amino acid sequence of SEQ ID NO: 22.

In some embodiments, the anti-IL-11 antibody or antigen-binding fragment thereof comprises a CH comprising or consisting of the amino acid sequence of SEQ ID NO: 22.

In some embodiments, the anti-IL-11 antibody or antigen-binding fragment thereof comprises a light chain constant region (CL) that has about 80%, about 85%, about 90%, about 95%, about 96%, about 97%, about 98%, about 99% or about 100% sequence identity to the amino acid sequence of SEQ ID NO: 24.

In some embodiments, the anti-IL-11 antibody or antigen-binding fragment thereof comprises a CL comprising or consisting of the amino acid sequence of SEQ ID NO: 24.

In some embodiments, the anti-IL-11 antibody or antigen-binding fragment thereof comprises: a CH that has about 80%, about 85%, about 90%, about 95%, about 96%, about 97%, about 98%, about 99% or about 100% sequence identity to the amino acid sequence of SEQ ID NO: 22; and a CL that has about 80%, about 85%, about 90%, about 95%, about 96%, about 97%, about 98%, about 99% or about 100% sequence identity to the amino acid sequence of SEQ ID NO: 24.

In some embodiments, the anti-IL-11 antibody or antigen-binding fragment thereof comprises a CH comprising or consisting of the amino acid sequence of SEQ ID NO: 22 and a CL comprising or consisting of the amino acid sequence of SEQ ID NO: 24.

In some embodiments, the antibody or antigen-binding fragment thereof comprises from N- to C-terminus the VH domain and the CH region, wherein the VH domain is operably linked to the CH domain.

In some embodiments, the antibody or antigen-binding fragment thereof comprises from N- to C-terminus the VL domain and the CL region, wherein the VL domain is operably linked to the CL region.

In some embodiments, the antibody or antigen-binding fragment thereof comprises from N- to C-terminus the VH domain and the CH region, wherein the VH domain is operably linked to the CH region; and wherein the antibody or antigen-binding fragment thereof comprises from N- to C-terminus the VL domain and the CL region, wherein the VL domain is operably linked to the CL region.

In some embodiments, the anti-IL-11 antibody or antigen-binding fragment thereof comprises a heavy chain that comprises the amino acid sequence of SEQ ID NO: 26, 27, 28, 29, or 30.

In some embodiments, the anti-IL-11 antibody or antigen-binding fragment thereof comprises a light chain that comprises the amino acid sequence of SEQ ID NO: 31, 32, or 33.

In some embodiments, the anti-IL-11 antibody or antigen-binding fragment thereof comprises:
(i) a heavy chain that comprises the amino acid sequence of SEQ ID NO: 26, and a light chain that comprises the amino acid sequence of SEQ ID NO: 31;
(ii) a heavy chain that comprises the amino acid sequence of SEQ ID NO: 27, and a light chain that comprises the amino acid sequence of SEQ ID NO: 31;
(iii) a heavy chain that comprises the amino acid sequence of SEQ ID NO: 27, and a light chain that comprises the amino acid sequence of SEQ ID NO: 33;
(iv) a heavy chain that comprises the amino acid sequence of SEQ ID NO: 28, and a light chain that comprises the amino acid sequence of SEQ ID NO: 32;
(v) a heavy chain that comprises the amino acid sequence of SEQ ID NO: 28, and a light chain that comprises the amino acid sequence of SEQ ID NO: 31;
(vi) a heavy chain that comprises the amino acid sequence of SEQ ID NO: 28, and a light chain that comprises the amino acid sequence of SEQ ID NO: 33;
(vii) a heavy chain that comprises the amino acid sequence of SEQ ID NO: 29, and a light chain that comprises the amino acid sequence of SEQ ID NO: 32;
(viii) a heavy chain that comprises the amino acid sequence of SEQ ID NO: 29, and a light chain that comprises the amino acid sequence of SEQ ID NO: 31;
(xi) a heavy chain that comprises the amino acid sequence of SEQ ID NO: 29, and a light chain that comprises the amino acid sequence of SEQ ID NO: 33;
(x) a heavy chain that comprises the amino acid sequence of SEQ ID NO: 30, and a light chain that comprises the amino acid sequence of SEQ ID NO: 32;
(xi) a heavy chain that comprises the amino acid sequence of SEQ ID NO: 30, and a light chain that comprises the amino acid sequence of SEQ ID NO: 31;
(xii) a heavy chain that comprises the amino acid sequence of SEQ ID NO: 30, and a light chain that comprises the amino acid sequence of SEQ ID NO: 33;
(xiii) a heavy chain that comprises the amino acid sequence of SEQ ID NO: 26, and a light chain that comprises the amino acid sequence of SEQ ID NO: 32; or
(xiv) a heavy chain that comprises the amino acid sequence of SEQ ID NO: 26, and a light chain that comprises the amino acid sequence of SEQ ID NO: 33.

In some embodiments, the anti-IL-11 antibody or antigen-binding fragment thereof is a monoclonal antibody.

In some embodiments, the anti-IL-11 antibody or antigen-binding fragment thereof is humanized.

In some embodiments, the anti-IL-11 antibody or antigen-binding fragment thereof binds to human IL-11 at a K_{D}≤0.50 nM.

In some embodiments, the anti-IL-11 antibody or antigen-binding fragment thereof is for use as a medicament.

In some aspects, the disclosure provides a polynucleotide encoding the VH and/or the VL of the anti-IL-11 antibody or antigen-binding fragment thereof as disclosed herein.

In some aspects, the disclosure provides a polynucleotide encoding the heavy chain and/or the light chain of the anti-IL-11 antibody or antigen-binding fragment thereof as disclosed herein.

In some aspects, the disclosure provides a set of polynucleotides comprising a first polynucleotide and a second polynucleotide, wherein the first polynucleotide encodes the VH of the anti-IL-11 antibody or antigen-binding fragment thereof as disclosed herein, and wherein the second polynucleotide encodes the VL of the anti-IL-11 antibody or antigen-binding fragment thereof as disclosed herein.

In some aspects, the disclosure provides a set of polynucleotides comprising a first polynucleotide and a second polynucleotide, wherein the first polynucleotide encodes the heavy chain of the anti-IL-11 antibody or antigen-binding fragment thereof as disclosed herein, and wherein the second polynucleotide encodes the light chain of the anti-IL-11 antibody or antigen-binding fragment thereof as disclosed herein.

In some embodiments, the disclosure provides a vector comprising the polynucleotide or the set of polynucleotides encoding the anti-IL-11 antibody or antigen-binding fragment thereof as disclosed herein.

In some aspects, the disclosure provides a host cell comprising the polynucleotide, the set of polynucleotides, or the vector encoding the anti-IL-11 antibody or antigen-binding fragment thereof as disclosed herein. In further embodiments, the host cell is a mammalian cell.

In some aspects, the disclosure provides a method for the production of the anti-IL-11 antibody or antigen-binding fragment thereof as disclosed herein, comprising the steps of: (a) cultivating the host cell under conditions suitable for expression of the anti-IL-11 antibody or antigen-binding fragment thereof; and (b) recovering the anti-IL-11 antibody or antigen-binding fragment thereof. In further embodiments, the method comprises the step of purifying the anti-IL-11 antibody or antigen-binding fragment thereof. In further embodiments, the method further comprises the step of formulating the anti-IL-11 antibody or antigen-binding fragment thereof into a pharmaceutical composition.

In some aspects, the disclosure further provides a diagnostic kit or diagnostic method comprising the anti-human IL-11 antibody or antigen-binding fragment thereof as disclosed herein.

In some aspects, the disclosure provides a pharmaceutical composition comprising the anti-IL-11 antibody or antigen-binding fragment thereof as disclosed herein and a pharmaceutically acceptable carrier. In some embodiments, the pharmaceutical composition further comprises one or more additional therapeutic agents.

In some aspects, the disclosure provides a method of treating idiopathic pulmonary fibrosis (IPF) or progressive pulmonary fibrosis (PPF) in a subject in need thereof by administering to the subject a therapeutically effective amount of the anti-IL-11 antibody or antigen-binding fragment thereof as disclosed herein or a pharmaceutical composition comprising the anti-IL-11 antibody or antigen-binding fragment thereof as disclosed herein.

In some aspects, the disclosure further provides an anti-IL-11 antibody or antigen-binding fragment thereof as disclosed herein, or a pharmaceutical composition comprising an anti-IL-11 antibody or antigen-binding fragment thereof as disclosed herein, for use in treating idiopathic pulmonary fibrosis (IPF) or progressive pulmonary fibrosis (PPF).

In some aspects, the disclosure further provides use of the anti-IL-11 antibody or antigen-binding fragment as disclosed herein, or a pharmaceutical composition comprising the anti-IL-11 antibody or antigen-binding fragment as disclosed herein, in manufacture of a medicament for treating idiopathic pulmonary fibrosis (IPF) or progressive pulmonary fibrosis (PPF).

In some aspects, the anti-IL-11 antibody or antigen-binding fragment is administered intravenously. In some aspects, the anti-IL-11 antibody or antigen-binding fragment is administered subcutaneously.

In some aspects, the disclosure further provides an anti-human IL-11 antibody or antigen-binding fragment, wherein when bound to human IL-11, the antibody or antigen-binding fragment binds to amino acid residues R15, D19, L23, R26, S27 L29, A30, D31, R33, R40, N50, L57, A58, R111, H161, L162, D165, W166, V168, R169, L172, L173, K175, or T176 as set forth in SEQ ID NO: 49, and wherein the antibody blocks binding of IL-11 to IL-11Rα.

### BRIEF DESCRIPTION OF THE DRAWINGS

The foregoing summary, as well as the following detailed description of the disclosure, will be better understood when read in conjunction with the appended figures. For the purpose of illustrating the disclosure, shown in the figures are embodiments which are presently preferred. It should be understood, however, that the disclosure is not limited to the precise arrangements, examples and instrumentalities shown.

FIG. 1 shows a representative concentration response curve of p-STAT3 inhibition for Antibodies A to O in human lung carcinoma cells (HTB183) cells stimulated with 50 ng/ml of IL-11. IC₅₀ values for Antibodies A to O are also provided. Each point represents two replicates+/−SEM.

FIG. 2 shows a representative concentration response curve of p-STAT3 inhibition for Antibody N in HTB183 cells stimulated with 50 ng/ml of human IL-11. An IC₅₀ value for Antibody N is also provided. Each point represents two replicates+/−SEM.

FIGS. 3A-3B show a representative concentration response curve and a plot of IL-11 signaling inhibition in vitro (FIG. 3A) and in vivo (FIG. 3B) for Antibody N. FIG. 3A shows a representative response curve of inhibition of IL-11 signaling for Antibody N in human hepatic stellate cells (HHSCs), while FIG. 3B shows a plot of inhibition of IL-11 signaling in mouse liver.

FIGS. 4A-4F show line graphs of stability parameter measurements for Antibody N at long-term storage conditions for 36 months. The parameters measured were percent low molecular weight species (LMW) (FIG. 4A), percent high molecular weight species (HMW) (FIG. 4B), percent acidic peak group (APG) (FIG. 4C), percent main peak (MP) (FIG. 4D), percent basic peak group (BPG) (FIG. 4E), and potency (FIG. 4F).

FIGS. 5A-5B show plots of alpha-actin-2 (ACTA2) activation (FIG. 5A) and Matrix metalloproteinase 2 (MMP2) secretion (FIG. 5B) in primary HHSCs stimulated with 5 ng/ml of TGFβ1 and then treated with Antibody N. Antibody N significantly decreased ACTA2 activation (FIG. 5A) and MMP2 secretion (FIG. 5B) compared to a control antibody. A reduction of MMP2 secretion was seen even as the concentration of Antibody N decreased (FIG. 5B). Plots are mean±SEM.

FIG. 6 shows a heat map of gene expression after treatment of Antibody N for high fat methionine choline deficient (HFMCD) mouse livers stimulated with IL-11. Mice were fed a HFMCD (with 0.1% methionine) diet for 6 weeks to induce liver steatosis and fibrosis. Antibody N treatment was initiated 1 week after the initiation of the HFMCD diet and continued for 5 weeks. Bar graphs are mean±SEM.

FIGS. 7A-7B show bar graphs of the effect of Antibody P on mRNA expression of a liver injury marker, alanine transaminase (ALT) (FIG. 7A), and a liver inflammation marker, C—C motif ligand 2 (CCL2) (FIG. 7B), in a HFMCD mouse model. Mice were fed a HFMCD (with 0.1% methionine) diet for 6 weeks to induce liver steatosis and fibrosis. Antibody P treatment was initiated 1 week after the initiation of the HFMCD diet and continued for 5 weeks. Bar graphs are mean±SEM.

FIGS. 8A-8C show bar graphs of the effect of Antibody P on mRNA expression of a profibrotic marker, collagen, type III, alpha 1 (COL3A1) (FIG. 8A), percent area of alpha-smooth muscle actin (α-SMA) (FIG. 8B), and percent area of PicroSirius Red (PSR) histomorphometry (SRM) staining (FIG. 8C) in HFMCD mice. Bar graphs are mean±SEM.

FIG. 9 shows a plot of molecule radius over time for Antibody A and Antibody N.

### DETAILED DESCRIPTION

This disclosure relates to anti-IL-11 antibodies or antigen-binding fragments thereof. The anti-IL-11 antibodies and antigen-binding fragments thereof disclosed herein exhibit improved stability (e.g, thermostability and/or long-term stability) as compared to a parental antibody IL-11 antibody with no significant changes observed for purity and potency. The present disclosure addresses the need for treatments of conditions modulated by IL-11 mediated signaling. In some aspects, the anti-IL-11 antibodies or antigen-binding fragments thereof of the disclosure are for diagnostic and/or therapeutic use including, for example in a subject in need thereof such as a human.

### Definitions

The terms, “antibody”, and “anti-IL-11 antibody”, are used herein interchangeably and encompass monoclonal antibodies (including full length monoclonal antibodies), multispecific antibodies (e.g., bispecific antibodies), antibodies with minor modifications such as N- or C-terminal truncations and antibody fragments such as variable domains and other portions of antibodies that exhibit a desired biological activity, e.g., IL-11 binding.

The generalized structure of antibodies or immunoglobulin is well known to those of skill in the art, these molecules are heterotetrametric glycoproteins, typically of about 150,000 daltons, composed of two identical light (L) chains and two identical heavy (H) chains. Each light chain is covalently linked to a heavy chain by one disulfide bond to form a heterodimer, and the heterotrimeric molecule is formed through a covalent disulfide linkage between the two identical heavy chains of the heterodimers. Although the light and heavy chains are linked together by one disulfide bond, the number of disulfide linkages between the two heavy chains varies by immunoglobulin isotype. Each heavy and light chain also has regularly spaced intrachain disulfide bridges. Each heavy chain has at the amino-terminus a variable domain (VH=variable heavy chain), followed by three or four constant domains (CH1, CH2, CH3, and CH4), as well as a hinge region between CH1 and CH2. Each light chain has two domains, an amino-terminal variable domain (VL=variable light chain) and a carboxy-terminal constant domain (CL). The VL domain associates non-covalently with the VH domain, whereas the CL domain is commonly covalently linked to the CH1 domain via a disulfide bond. Particular amino acid residues are believed to form an interface between the light and heavy chain variable domains (Chothia et al., 1985, J. Mol. Biol. 186:651-663, Vargas-Madrazo E, Paz-García E. J Mol Recognit. 2003; 16(3): 113-120). The variable domains are also referred herein as variable regions, and the constant domains as constant regions.

Certain domains within the variable domains differ extensively between different antibodies i.e., are “hypervariable.” These hypervariable domains contain residues that are directly involved in the binding and specificity of each particular antibody for its specific antigenic determinant. Hypervariability, both in the light chain and the heavy chain variable domains, is concentrated in three segments known as complementarity determining regions (CDRs) or hypervariable loops (HVLs). CDRs are defined by sequence comparison in Kabat et al., 1991, In: Sequences of Proteins of Immunological Interest, 5th Ed. Public Health Service, National Institutes of Health, Bethesda, Md., whereas HVLs are structurally defined according to the three-dimensional structure of the variable domain, as described by Chothia and Lesk, 1987, J. Mol. Biol. 196:901-917. Where these two methods result in slightly different identifications of a CDR, the structural definition is preferred. As defined by Kabat, CDR-L1 is positioned at about residues 24-34, CDR-L2, at about residues 50-56, and CDR-L3, at about residues 89-97 in the light chain variable domain; CDR-H1 is positioned at about residues 31-35, CDR-H2 at about residues 50-65, and CDR-H3 at about residues 95-102 in the heavy chain variable domain. IMGT and NORTH provide alternative definitions of the CDRs (see, Lefranc MP. Unique database numbering system for immunogenetic analysis. Immunol Today (1997) 18:509; and North B, Lehmann A, Dunbrack R L J. A new clustering of antibody CDR loop conformations. J Mol Biol. (2011) 406:228-56). Additionally, CDRs may be defined per the Chemical Computing Group (CCG) numbering (Almagro et al., Proteins 2011; 79:3050-3066 and Maier et al, Proteins 2014; 82:1599-1610). The CDR1, CDR2, CDR3 of the heavy and light chains therefore define the unique and functional properties specific for a given antibody. Except where indicated otherwise, the present invention has been disclosed with reference to the CDRs defined according to Kabat, though it is to be understood that the invention could likewise be determined with reference to other CDR numbering schemes, such as but not limited to Chothia, CCG, IMGT or NORTH as disclosed herein.

The three CDRs within each of the heavy and light chains are separated by framework regions (FR), which contain sequences that tend to be less variable. From the amino terminus to the carboxy terminus of the heavy and light chain variable domains, the FRs and CDRs are arranged in the order: FR1, CDR1, FR2, CDR2, FR3, CDR3, and FR4. The largely β-sheet configuration of the FRs brings the CDRs within each of the chains into close proximity to each other as well as to the CDRs from the other chain. The resulting conformation contributes to the antigen binding site (see, e.g., Kabat et al., 1991, NIH Publ. No. 91-3242, Vol. I, pages 647-669), although not all CDR residues are necessarily directly involved in antigen binding. Those skilled in the art can routinely determine which residues comprise a particular CDR given the variable region amino acid sequence of the antibody. The CDR1, CDR2, CDR3 of the heavy and light chains therefore define the unique and functional properties specific for a given antibody.

FR residues and Ig constant domains are generally not directly involved in antigen binding but contribute to antigen binding and/or mediate antibody effector function. Some FR residues are thought to have a significant effect on antigen binding in at least three ways: by noncovalently binding directly to an epitope, by interacting with one or more CDR residues, and by affecting the interface between the heavy and light chains. The constant domains are not directly involved in antigen binding but mediate various Ig effector functions, such as participation of the antibody in antibody-dependent cellular cytotoxicity (ADCC), complement-dependent cytotoxicity (CDC) and antibody-dependent cellular phagocytosis (ADCP).

The light chains of vertebrate immunoglobulins are assigned to one of two clearly distinct classes, kappa (κ) and lambda (λ), based on the amino acid sequence of the constant domain. By comparison, the heavy chains of mammalian immunoglobulins are assigned to one of five major classes, according to the sequence of the constant domains: IgA, IgD, IgE, IgG, and IgM. IgG and IgA are further divided into subclasses (isotypes), e.g., IgG1, IgG2, IgG3, IgG4, IgA1, and IgA2, respectively. The heavy chain constant domains that correspond to the different classes of immunoglobulins are called α, δ, ε, γ, and μ, respectively. The subunit structures and three-dimensional configurations of the classes of native immunoglobulins are well known.

The term “monoclonal antibody” refers to an antibody obtained from a substantially homogenous population of antibody molecules, i.e., the individual antibodies comprising the population are identical except for possible well-known alterations such as removal of C-terminal lysine from the antibody heavy chain or post-translational modifications such as amino acid isomerization or deamidation, methionine oxidation or asparagine or glutamine deamidation that may be present. Monoclonal antibodies typically bind one antigenic epitope. A bispecific monoclonal antibody binds at least two distinct antigenic epitopes. A monoclonal antibody may be monospecific or multispecific such as bispecific, monovalent, bivalent or multivalent. It should be understood that monoclonal antibodies can be made by any technique or methodology known in the art; including e.g., the hybridoma method (Kohler et al., 1975, Nature 256:495), or recombinant DNA methods known in the art (see, e.g., U.S. Pat. No. 4,816,567), or methods of isolation of monoclonal recombinantly produced using phage antibody libraries, using techniques described in Clackson et al., 1991, Nature 352:624-628, and Marks et al., 1991, J. Mol. Biol. 222:581-597.

Chimeric antibodies consist of the heavy and light chain variable regions of an antibody from one species (e.g., a non-human mammal such as a mouse) and the heavy and light chain constant regions of another species (e.g., human) antibody and can be obtained by linking the DNA sequences encoding the variable regions of the antibody from the first species (e.g., mouse) to the DNA sequences for the constant regions of the antibody from the second (e.g. human) species and transforming a host with an expression vector containing the linked sequences to allow it to produce a chimeric antibody. Alternatively, the chimeric antibody also could be one in which one or more regions or domains of the heavy and/or light chain is identical with, homologous to, or a variant of the corresponding sequence in a monoclonal antibody from another immunoglobulin class or isotype, or from a consensus or germline sequence. Chimeric antibodies can include fragments of such antibodies, provided that the antibody fragment exhibits the desired biological activity of its parent antibody, for example binding to the same epitope (see, e.g., U.S. Pat. No. 4,816,567; and Morrison et al., 1984, Proc. Natl. Acad. Sci. USA 81:6851-6855).

The terms “antibody fragment”, “antigen-binding fragment”, “anti-IL-11 antibody fragment”, “anti-IL-11 antibody fragment”, “engineered anti-IL-11 antibody fragment” refer to a portion of a full-length anti-IL-11 antibody, in which a variable region or a functional capability is retained, for example, IL-11 binding. Examples of antibody fragments include, but are not limited to, a Fab, Fab′, F(ab′)₂, Fd, Fv, scFv and scFv-Fc fragment, a diabody, a linear antibody, a single-chain antibody, a minibody, a diabody formed from antibody fragments, and multispecific antibodies formed from antibody fragments.

Antibody fragments can be obtained for example by treating full-length antibodies treated with enzymes such as papain or pepsin to generate useful antibody fragments. Papain digestion is used to produce two identical antigen-binding antibody fragments called “Fab” fragments, each with a single antigen-binding site, and a residual “Fc” fragment. The Fab fragment also contains the constant domain of the light chain and the CH1 domain of the heavy chain. Pepsin treatment yields a F(ab′)₂ fragment that has two antigen-binding sites and is still capable of cross-linking antigen.

Another example of antibody fragments according to the disclosure are Fab′ fragments. Fab′ fragments differ from Fab fragments by the presence of additional residues including one or more cysteines from the antibody hinge region at the C-terminus of the CH1 domain. F(ab′)₂ antibody fragments are pairs of Fab′ fragments linked by cysteine residues in the hinge region. Other chemical couplings of antibody fragments are also known.

A “Fv” fragment contains a complete antigen-recognition and binding site consisting of a dimer of one heavy and one light chain variable domain in tight, non-covalent association. In this configuration, the three CDRs of each variable domain interact to define an antigen-biding site on the surface of the VH-VL dimer. Collectively, the six CDRs confer antigen-binding specificity to the antibody.

Antibody fragments may also include “single-chain Fv” or “scFv” fragments. A “single-chain Fv” or “scFv” antibody fragment is a single chain Fv variant comprising the VH and VL domains of an antibody where the domains are present in a single polypeptide chain. The single chain Fv is capable of recognizing and binding antigen. The scFv polypeptide may optionally also contain a polypeptide linker positioned between the VH and VL domains in order to facilitate formation of a desired three-dimensional structure for antigen binding by the scFv (see, e.g., Pluckthun, 1994, In The Pharmacology of Monoclonal Antibodies, Vol. 113, Rosenburg and Moore eds., Springer-Verlag, New York, pp. 269-315).

Antibody fragments may also form tandem Fd segments, which comprise a pair of tandem Fd segments (VH-CH1-VH-CH1) to form a pair of antigen binding regions. These “linear antibodies” can be bispecific or monospecific as described in, for example, Zapata et al. 1995, Protein Eng. 8(10): 1057-1062.

The term “human antibody” as used herein includes antibodies or fragments thereof derived from human germline immunoglobulin sequences. The term “human antibody” is not intended to include antibodies in which CDR sequences derived from the germline of another (mammalian) species, such as a mouse, rat or rabbit, have been grafted onto human framework sequences. Thus, as used herein, the term “human antibody” refers to an antibody or fragment thereof in which every part of the protein (e.g., CDR, framework, CL, CH domains (e.g., CH1, CH2, CH3), hinge, VL, VH) is substantially non-immunogenic in humans, with only minor sequence changes or variations as further described herein below.

Technologies for creating such a “human antibody” have been described and include without being limiting phage display or use of transgenic animals (www.Ablexis.com/technology-alivamab.php; WO 90/05144; D. Marks, H. R. Hoogenboom, T. P. Bonnert, J. McCafferty, A. D. Griffiths and G. Winter (1991) “By-passing immunization. Human antibodies from V-gene libraries displayed on phage.” J. Mol. Biol., 222, 581-597; Knappik et al., J. Mol. Biol. 296:57-86, 2000; S. Carmen and L. Jermutus, “Concepts in antibody phage display”. Briefings in Functional Genomics and Proteomics 2002 1(2): 189-203; Lonberg N, Huszar D. “Human antibodies from transgenic mice”. Int Rev Immunol. 1995; 13(1): 65-93; Brüggemann M, Taussig M J. “Production of human antibody repertoires in transgenic mice”. Curr Opin Biotechnol. 1997 August; 8(4): 455-8.).

Thus, a human antibody is distinct from e.g., a chimeric or humanized antibody. It is pointed out that a human antibody can be produced by a non-human animal or prokaryotic or eukaryotic cell that is capable of expressing functionally rearranged human immunoglobulin (e.g., heavy chain and/or light chain) genes.

In some aspects, an anti-IL-11 antibody of the disclosure is a humanized antibody or antibody fragment thereof. A humanized antibody or a humanized antibody fragment is a specific type of chimeric antibody which includes an immunoglobulin amino acid sequence variant, or fragment thereof, which is capable of binding to a predetermined antigen and which, comprises one or more FRs having substantially the amino acid sequence of a human immunoglobulin and one or more CDRs having substantially the amino acid sequence of a non-human immunoglobulin. This non-human amino acid sequence often referred to as an “import” sequence is typically taken from an “import” antibody domain, particularly a variable domain. In general, a humanized antibody includes at least the CDRs or HVLs of a non-human antibody, inserted between the FRs of a human heavy or light chain variable domain. Methods of humanization of antibodies are for example described by Almagro et al., (2008) Frontiers in Bioscience 13, 1619-1633, or in WO 2012/092374A2.

The chimeric, humanized or human antibodies or antigen-binding fragments thereof of the present disclosure may further be engineered. Such engineering includes without limitation the removal or exchange of undesired amino acids, for example to reduce immunogenicity in humans, or to avoid deamidation, undesirable charges or lipophilicity or non-specific binding. Such removal or exchange of undesired amino acids can, for example, be introduced by random or site-specific mutagenesis in vitro or by somatic mutation in vivo. Moreover, in connection with chimeric or humanized antibodies, it will be understood that certain mouse FR residues may be retained in an antibody or fragment thereof.

In some aspects, an anti-IL-11 antibody comprises substantially all of at least one, and typically two, variable domains (such as contained, for example, in Fab, Fab′, F(ab′)₂, and Fv fragments). In some aspects, an anti-IL-11 antibody also includes at least a portion of an immunoglobulin Fc region, typically that of a human immunoglobulin. Ordinarily, the antibody will contain both the light chain as well as at least the variable domain of a heavy chain. The antibody also may include one or more of the CH1, hinge, CH2, CH3, and/or CH4 regions of the heavy chain, as appropriate.

In some aspects, an anti-IL-11 antibody can be selected from any class of immunoglobulins, including IgM, IgG, IgD, IgA and IgE, and any isotype, including IgG1, IgG2, IgG3, IgG4, IgA1 and IgA2. An alternative anti-IL-11 antibody can comprise sequences from more than one immunoglobulin class or isotype, and selecting particular modified or unmodified constant domains to optimize desired effector functions is within the ordinary skill in the art.

For example, the Fc region of an antibody mediates its serum half-life and effector functions, such as complement-dependent cytotoxicity (CDC), antibody-dependent cellular cytotoxicity (ADCC) and antibody-dependent cell phagocytosis (ADCP). Fc engineering can be employed to optimize antibody properties suited to the pharmacology activity required of them. Where such cytotoxic activity is not desirable, such as targeting an immune cell in the treatment of cancer, the constant domain may be of isotype with reduced effector function, such as IgG4, and/or be modified with known modifications that reduce effector function. Where such cytotoxic activity is desirable, such as for destruction of a targeted tumor cell, the constant domain may be of isotype with increased effector function and/or be modified with known modifications to increase effector function. Several mutations are known to either reduce or increase effector function. See, e.g., “The future of antibodies as cancer drugs” Janice M Reichert, Eugen Dhimolea, Drug Discov Today (2012) Septmeber; 17(17-18): 954-63, “Antibody Drug Discovery” (Volume 4 of Molecular medicine and medicinal chemistry) Clive R. Wood, World Scientific, 2012; “FcγR requirements leading to successful immunotherapy” Immunol Rev. (2015) November; 268(1): 104-22.

In some aspects, the constant domain of an antibody of the present disclosure is IgG4Pro, which has one replacement mutation (Ser228Pro) that prevents Fab-arm exchanging. In some aspects, the constant domain of an antibody of the present disclosure is IgG1, which optionally has two mutations in the constant region, Leu234Ala and Leu235Ala to reduce effector function.

The FRs and CDRs, or HVLs, of an engineered anti-IL-11 antibody or antigen-binding fragment thereof need not correspond precisely to the parental sequences. For example, a parental sequence may be altered (e.g., mutagenized) by substitution, insertion or deletion such that the resulting amino acid residue is no longer identical to the original residue in the corresponding position in either parental sequence, but the antibody nevertheless retains the function of binding to IL-11. Such alteration typically will not be extensive and will be conservative alterations. Usually, at least 75% of the engineered antibody residues will correspond to those of the parental sequences, more often at least 90%, and most frequently greater than 95%, or greater than 98% or greater than 99%.

Immunoglobulin residues that affect the interface between heavy and light chain variable regions (“the VL-VH interface”) are those that affect the proximity or orientation of the two chains with respect to one another. Certain residues that may be involved in interchain interactions include VL residues 34, 36, 38, 44, 46, 87, 89, 91, 96, and 98 and VH residues 35, 37, 39, 45, 47, 91, 93, 95, 100, and 103 (utilizing the numbering system set forth in Kabat et al., Sequences of Proteins of Immunological Interest (National Institutes of Health, Bethesda, Md., 1987)). U.S. Pat. No. 6,407,213 also discusses that residues such as VL residues 43 and 85, and VH residues 43 and 60 also may be involved in this interaction. While these residues are indicated for human IgG only, they are applicable across species. Important antibody residues that are reasonably expected to be involved in interchain interactions are selected for substitution into the consensus sequence.

The terms “consensus sequence” and “consensus antibody” refer to an amino acid sequence which comprises the most frequently occurring amino acid residue at each location in all immunoglobulins of any particular class, isotype, or subunit structure, e.g., a human immunoglobulin variable domain. The consensus sequence may be based on immunoglobulins of a particular species or of many species. A “consensus” sequence, structure, or antibody is understood to encompass a consensus human sequence as described in certain embodiments, and to refer to an amino acid sequence which comprises the most frequently occurring amino acid residues at each location in all human immunoglobulins of any particular class, isotype, or subunit structure. Thus, the consensus sequence contains an amino acid sequence having at each position an amino acid that is present in one or more known immunoglobulins, but which may not exactly duplicate the entire amino acid sequence of any single immunoglobulin. The variable region consensus sequence is not obtained from any naturally produced antibody or immunoglobulin. Kabat et al., 1991, Sequences of Proteins of Immunological Interest, 5th Ed. Public Health Service, National Institutes of Health, Bethesda, Md., and variants thereof. The FRs of heavy and light chain consensus sequences, and variants thereof, provide useful sequences for the preparation of human or humanized anti-IL-11 antibodies. See, for example, U.S. Pat. Nos. 6,037,454 and 6,054,297.

An “isolated” antibody is one that has been identified and separated and/or recovered from a component of its natural environment or from a cell culture from which it was expressed. An isolated antibody or antibody fragment may have one or more co- or post-translational modifications that arise during production, purification, and/or storage of the antibody or antibody fragment. Contaminant components of the antibody's natural environment are those materials that may interfere with diagnostic or therapeutic uses of the antibody, and can be enzymes, hormones, or other proteinaceous or non-proteinaceous solutes. In some aspects, the antibody will be purified to at least greater than 95% isolation by weight of antibody, for example purified to at least greater than 95%, 96%, 97%, 98%, or 99%.

An isolated antibody includes an antibody in situ within recombinant cells in which it is produced, since at least one component of the antibody's natural environment will not be present. Ordinarily however, an isolated antibody will be prepared by at least one purification step in which the recombinant cellular material is removed.

“Multispecific” refers to a protein, such as an antibody, that specifically binds two or more distinct antigens or two or more distinct epitopes within the same antigen.

“Bispecific” refers to a protein, such as an antibody, that specifically binds two distinct antigens or two distinct epitopes within the same antigen.

In some embodiments, the antibody that specifically binds IL-11 or the antigen-binding fragment thereof of the disclosure is a bispecific antibody. In some embodiments, the antibody or the antigen-binding fragment thereof of the disclosure is a multispecific antibody. The monospecific antibodies that specifically bind IL-11 provided herein may be engineered into bispecific antibodies, which are also encompassed within the scope of the disclosure.

Full-length bispecific antibodies may be generated for example using Fab arm exchange (e.g., half-molecule exchange, exchanging one heavy chain-light chain pair) between two monospecific bivalent antibodies by introducing substitutions at the heavy chain CH3 interface in each half molecule to favor heterodimer formation of two antibody half molecules having distinct specificity either in vitro in cell-free environment or using co-expression. The Fab arm exchange reaction is the result of a disulfide-bond.

Bispecific antibodies may also be generated using designs such as the Triomab/Quadroma (Trion Pharma/Fresenius Biotech), Knob-in-Hole (Genentech), CrossMAbs (Roche) and the electrostatically-induced CH3 interaction (Chugai, Amgen, NovoNordisk, Oncomed), the LUZ-Y (Genentech), the Strand Exchange Engineered Domain body (SEEDbody) (EMD Serono), the Biclonic (Merus) and as DuoBody® Products (Genmab A/S).

As used herein, the terms “identical” or “percent identity,” in the context of two or more nucleic acids or polypeptide sequences, refer to two or more sequences or subsequences that are the same or have a specified percentage of nucleotides or amino acid residues that are the same, when compared and aligned for maximum correspondence. To determine the percent identity, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions/total # of positions (e.g., overlapping positions)×100). In some embodiments, the two sequences that are compared are the same length after gaps are introduced within the sequences, as appropriate (e.g., excluding additional sequence extending beyond the sequences being compared). For example, when variable region sequences are compared, the leader and/or constant domain sequences are not considered. For sequence comparisons between two sequences, a “corresponding” CDR refers to a CDR in the same location in both sequences (e.g., CDR-H1 of each sequence).

The determination of percent identity or percent similarity between two sequences can be accomplished using a mathematical algorithm. A preferred, non-limiting example of a mathematical algorithm utilized for the comparison of two sequences is the algorithm of Karlin and Altschul, 1990, Proc. Natl. Acad. Sci. USA 87:2264-2268, modified as in Karlin and Altschul, 1993, Proc. Natl. Acad. Sci. USA 90:5873-5877. Such an algorithm is incorporated into the NBLAST and XBLAST programs of Altschul et al., 1990, J. Mol. Biol. 215:403-410. BLAST nucleotide searches can be performed with the NBLAST program, score=100, wordlength=12, to obtain nucleotide sequences homologous to a nucleic acid encoding a protein of interest. BLAST protein searches can be performed with the XBLAST program, score=50, wordlength=3, to obtain amino acid sequences homologous to protein of interest. To obtain gapped alignments for comparison purposes, Gapped BLAST can be utilized as described in Altschul et al., 1997, Nucleic Acids Res. 25:3389-3402. Alternatively, PSI-Blast can be used to perform an iterated search which detects distant relationships between molecules (ld.). When utilizing BLAST, Gapped BLAST, and PSI-Blast programs, the default parameters of the respective programs (e.g., XBLAST and NBLAST) can be used. Another preferred, non-limiting example of a mathematical algorithm utilized for the comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Additional algorithms for sequence analysis are known in the art and include ADVANCE and ADAM as described in Torellis and Robotti, 1994, Comput. Appl. Biosci. 10:3-5; and FASTA described in Pearson and Lipman, 1988, Proc. Natl. Acad. Sci. USA 85:2444-8. Within FASTA, ktup is a control option that sets the sensitivity and speed of the search. If ktup=2, similar regions in the two sequences being compared are found by looking at pairs of aligned residues; if ktup=1, single aligned amino acids are examined. ktup can be set to 2 or 1 for protein sequences, or from 1 to 6 for DNA sequences. The default if ktup is not specified is 2 for proteins and 6 for DNA. Alternatively, protein sequence alignment may be carried out using the CLUSTAL W algorithm, as described by Higgins et al., 1996, Methods Enzymol. 266:383-402.

For diagnostic as well as therapeutic monitoring purposes, the antibodies or antigen-binding fragment thereof of the disclosure also may be conjugated to a label, either a label alone or a label and an additional second agent (prodrug, chemotherapeutic agent and the like). A label, as distinguished from the other second agents refers to an agent that is a detectable compound or composition and it may be conjugated directly or indirectly to an anti-IL-11 antibody or antigen-binding fragment thereof of the present disclosure. The label may itself be detectable (e.g., radioisotope labels or fluorescent labels) or, in the case of an enzymatic label, may catalyze chemical alteration of a substrate compound or composition that is detectable. Labeled anti-IL-11 antibodies or antigen-binding fragments thereof can be prepared and used in various applications including in vitro and in vivo diagnostics.

In various aspects of the present disclosure one or more domains of the anti-IL-11 antibodies or antigen-binding fragments thereof will be recombinantly expressed. Such recombinant expression may employ one or more control sequences, i.e., polynucleotide sequences necessary for expression of an operably linked coding sequence in a particular host organism. The control sequences suitable for use in prokaryotic cells include, for example, promoter, operator, and ribosome binding site sequences. Eukaryotic control sequences include, but are not limited to, promoters, polyadenylation signals, and enhancers. These control sequences can be utilized for expression and production of anti-IL-11 antibodies or antigen-binding fragments thereof in prokaryotic and eukaryotic host cells.

A nucleic acid sequence is “operably linked” when it is placed into a functional relationship with another nucleic acid sequence. For example, a nucleic acid presequence or secretory leader is operably linked to a nucleic acid encoding a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading frame. However, enhancers are optionally contiguous. Linking can be accomplished by ligation at convenient restriction sites. If such sites do not exist, synthetic oligonucleotide adaptors or linkers can be used.

As used herein, the expressions “cell”, “cell line”, and “cell culture” are used interchangeably and all such designations include the progeny thereof. Thus, “transformants” and “transformed cells” include the primary subject cell and cultures derived therefrom without regard for the number of transfers, which may for example have been transfected with one or more expression vectors encoding one or more amino acids sequences of an antibody or antigen-binding fragment thereof of the present disclosure.

The term “mammal” for purposes of treatment according to the disclosure refers to any animal classified as a mammal, including humans, domesticated and farm animals, and zoo, sports, or pet animals, such as dogs, horses, cats, cows, and the like. Preferably, the mammal is a human.

A “disorder”, as used herein, is any condition that would benefit from treatment with an anti-IL-11 antibody or antigen-binding fragment thereof described herein. This includes chronic and acute disorders or diseases including those pathological conditions that predispose the mammal to the disorder in question. Non-limiting examples of fibrosis include pulmonary diseases or disorders and liver diseases or disorders.

As used herein, the term “prevent” or “prevention” refers to prophylactic or preventative measures that prevent and/or slow the development of a targeted pathologic condition or disorder. Thus, those in need of prevention include those prone to have or susceptible to the disorder. In some embodiments, a disease or disorder (preferably a IL-11 pathway disorder) is successfully prevented according to the methods provided herein if the patient develops, transiently or permanently, e.g., fewer or less severe symptoms associated with the disease or disorder, or a later onset of symptoms associated with the disease or disorder, than a patient who has not been subject to the methods of the disclosure.

As used herein, the term “pulmonary diseases or disorders” refers to any disease or disorder that affects the pulmonary system.

As used herein, the term “liver disease” or “liver disorder” refers to any disease or disorder that affects the liver. In some embodiments, the disease or disorder is acute liver injury (ALI), acute liver failure, acute liver disease, chronic liver disease, liver damage, hepatitis (e.g., viral hepatitis, alcoholic hepatitis), steatotic liver disease (SLD), metabolic dysfunction-associated steatotic liver disease (MASLD), liver ischemia-reperfusion injury (IRI) (e.g., ‘warm’ ischemia-reperfusion (WIR)), radiation-induced liver disease (RILD), drug-induced liver injury (DILI), idiosyncratic drug-induced liver injury (IDILI), autoimmune liver injury, cholestatic liver disease, HIV-associated liver injury, or cancer.

The term “cirrhosis” refers to scarring (e.g., fibrosis) of the liver.

The term “metabolic dysfunction-associated steatotic liver disease” (MASLD; formerly non-alcoholic fatty liver disease (NAFLD)) refers to a condition which is one cause of a fatty liver, occurring when fat is deposited in the liver not due to excessive alcohol use. MASLD is related to insulin resistance and the metabolic syndrome and may respond to treatments originally developed for other insulin-resistant states (e.g., diabetes mellitus type 2) such as weight loss. MASLD can be sub-classified as MASH and nonalcoholic fatty liver (MASL; formerly non-alcoholic fatty liver (NAFL)). MASH is the more extreme form of MASLD and is regarded as a major cause of cirrhosis of the liver of unknown cause.

Diseases and disorders characterized by fibrosis in accordance with the present invention include but are not limited to: respiratory conditions such as pulmonary fibrosis, cystic fibrosis, idiopathic pulmonary fibrosis (IPF), progressive massive fibrosis, scleroderma, obliterative bronchiolitis, Hermansky-Pudlak syndrohivme, asbestosis, silicosis, chronic pulmonary hypertension, AIDS associated pulmonary hypertension, sarcoidosis, tumor stroma in lung disease, and asthma; chronic liver disease, primary biliary cirrhosis (PBC), schistosomal liver disease, liver cirrhosis, steatohepatitis, metabolic dysfunction-associated steatohepatitis (MASH), early-stage MASH, late-stage MASH, alcoholic steatohepatitis, steatosis, metabolic dysfunction-associated steatotic liver disease (MASLD), type 1 glycogen storage disease, alcohol-induced liver fibrosis, alcohol-induced cirrhosis, cystic fibrosis-associated liver disease (CFLD), alcohol related liver disease, alpha 1 antitrypsin deficiency, autoimmune hepatitis, benign liver tumors, hemochromatosis, hepatitis A, hepatitis B, hepatitis C, hepatocellular carcinoma, liver cysts, Wilson's disease, Alagille syndrome, biliary atresia, congestive hepatopathy, drug induced liver injury, focal nodular hyperplasia, ischemic hepatitis, lysosomal acid lipase deficiency, polycystic liver disease, progressive familial intrahepatic cholestasis, and veno-occlusive disease; pancreatic conditions such as chronic pancreatitis and pancreatic fibrosis; cardiovascular conditions such as hypertrophic cardiomyopathy, dilated cardiomyopathy (DCM), fibrosis of the atrium, atrial fibrillation, fibrosis of the ventricle, ventricular fibrillation, myocardial fibrosis, hepatotoxicity, Brugada syndrome, myocarditis, endomyocardial fibrosis, myocardial infarction, fibrotic vascular disease, hypertensive heart disease, arrhythmogenic right ventricular cardiomyopathy (ARVC), tubulointerstitial and glomerular fibrosis, atherosclerosis, varicose veins, cerebral infarcts; neurological conditions such as gliosis and Alzheimer's disease; muscular dystrophy such as Duchenne muscular dystrophy (DMD) or Becker's muscular dystrophy (BMD); gastrointestinal conditions such as Crohn's disease, microscopic colitis and primary sclerosing cholangitis (PSC); skin conditions such as scleroderma, nephrogenic systemic fibrosis and cutis keloid; arthrofibrosis; Dupuytren's contracture; mediastinal fibrosis; retroperitoneal fibrosis; myelofibrosis; Peyronie's disease; adhesive capsulitis; kidney disease (e.g., renal fibrosis, nephritic syndrome, Alport's syndrome, HIV associated nephropathy, polycystic kidney disease, Fabry's disease, diabetic nephropathy, chronic glomerulonephritis, nephritis associated with systemic lupus, kidney interstitial fibrosis (IF)); kidney injury e.g. acute kidney injury/renal failure; nephrotoxicity; progressive systemic sclerosis (PSS); chronic graft versus host disease; diseases/disorders of the eye and associated processes, such as Grave's opthalmopathy, epiretinal fibrosis (e.g., diabetic retinopathy (DR)), retinal fibrosis, idiopathic premacular fibrosis, corneal fibrosis, glaucoma, subretinal fibrosis (e.g. associated with macular degeneration (e.g., wet or dry age-related macular degeneration (AMD)), macular edema, drusen formation, choroidal neovascularization (CNV), post-surgical fibrosis (e.g. of the posterior capsule following cataract surgery, or of the bleb following trabeculectomy for glaucoma), conjunctival fibrosis, subconjunctival fibrosis; arthritis; fibrotic pre-neoplastic and fibrotic neoplastic disease; and fibrosis induced by chemical or environmental insult (e.g., cancer chemotherapy, pesticides, radiation/cancer radiotherapy).

The terms “specifically binds” or “specific binding” in the context of an antibody or antigen-binding fragment thereof, refers to an antibody or antigen-binding fragment thereof that associates more frequently, more rapidly, with greater duration, with greater affinity, with greater avidity or with some combination of the above, to an antigen or an epitope within the antigen than with an unrelated antigen. In certain embodiments, an antibody or antigen-binding fragment thereof specifically binds to an antigen or epitope within an antigen with a K_{D} of about 0.1 mM or less, preferably less than about 1 μM. Because of the sequence identity between homologous proteins in different species, or variants of a protein within a single species, specific binding can include an antibody or antigen-binding fragment thereof that recognizes a protein in more than one species (e.g., human IL-11 and mouse IL-11). It is understood that, in certain embodiments, an antibody or antigen-binding fragment thereof that specifically binds a first protein may or may not specifically bind a second protein. As such, “specific binding” does not necessarily require (although it can include) exclusive binding, i.e. binding to a single protein. Thus, an antibody or antigen-binding fragment thereof may, in certain embodiments, specifically bind more than one protein.

Methods for determining whether two molecules specifically bind a protein are described herein or a known in the art and include, for example, equilibrium dialysis, surface plasmon resonance, and the like. In some embodiments, specific binding is characterized by a KD of about 1×10⁻⁸ M (10 nM) or less according to the affinity binding method described in the Examples section herein. In some embodiments, specific binding is characterized by a KD of about 5×10⁻⁹ M (5 nM) or less according to the affinity binding method described in the Examples section herein.

The term “subcutaneous administration” refers to introduction of a drug, for example an anti-IL-11 antibody or antigen-binding fragment thereof of the disclosure, under the outer layers of the skin (called epidermis and dermis) of a subject such as an animal or human patient, preferable within a pocket between the skin and underlying tissue, by relatively slow, sustained delivery from a drug receptacle. Pinching or drawing the skin up and away from underlying tissue may create the pocket.

The term “subcutaneous infusion” refers to introduction of a drug, for example an anti-IL-11 antibody or antigen-binding fragment thereof of the disclosure, under the skin of a subject, preferably within a pocket between the skin and underlying tissue, by relatively slow, sustained delivery from a drug receptacle for a period of time including, but not limited to, 30 minutes or less, or 90 minutes or less. Optionally, the infusion may be made by subcutaneous implantation of a drug delivery pump implanted under the skin of the subject, wherein the pump delivers a predetermined amount of drug for a predetermined period of time, such as 30 minutes, 90 minutes, or a time period spanning the length of the treatment regimen.

The term “subcutaneous bolus” refers to drug administration beneath the skin of a subject, where bolus drug delivery is less than approximately 15 minutes; in some aspects, less than 5 minutes, and in some aspects, less than 60 seconds. In some aspects, administration is within a pocket between the skin and underlying tissue, where the pocket may be created by pinching or drawing the skin up and away from underlying tissue. For example, “subcutaneous bolus” refers to the administration of an anti-IL-11 antibody or antigen-binding fragment thereof of the disclosure to a subject in less than approximately 15 minutes; in some aspects, less than 5 minutes, and in some aspects, less than 60 seconds.

To “prevent” refers to prophylactic or preventative measures that prevent and/or slow the development of a targeted pathologic condition or disorder. Thus, those in need of prevention include those prone to have or susceptible to the disorder. In some embodiments, a disease or disorder (preferably a IL-11 pathway disorder) is successfully prevented according to the methods provided herein if the patient develops, transiently or permanently, e.g., fewer or less severe symptoms associated with the disease or disorder, or a later onset of symptoms associated with the disease or disorder, than a patient who has not been subject to the methods of the disclosure.

The term “therapeutically effective amount” is used to refer to an amount of an anti-IL-11 antibody or antigen-binding fragment thereof that relieves or ameliorates one or more of the symptoms of the disorder being treated. In doing so, it is that amount that has a beneficial patient outcome. Efficacy can be measured in conventional ways, depending on the condition to be treated.

The terms “treatment” and “therapy” and the like, as used herein, are meant to include therapeutic as well as prophylactic, or suppressive measures for a disease or disorder leading to any clinically desirable or beneficial effect, including but not limited to alleviation or relief of one or more symptoms, regression, slowing or cessation of progression of the disease or disorder. Thus, for example, the term treatment includes the administration of an anti-IL-11 antibody or antigen-binding fragment thereof prior to or following the onset of a symptom of a disease or disorder thereby preventing or removing one or more signs of the disease or disorder. As an example, the term includes the administration of an anti-IL-11 antibody or antigen-binding fragment thereof after clinical manifestation of the disease to combat the symptoms of the disease. Further, administration of an anti-IL-11 antibody or antigen-binding fragment thereof after onset and after clinical symptoms have developed where administration affects clinical parameters of the disease or disorder, such as the degree of tissue injury or the amount or extent of metastasis, whether or not the treatment leads to amelioration of the disease, comprises “treatment” or “therapy” as used herein. Moreover, as long as the compositions of the disclosure either alone or in combination with another therapeutic agent alleviate or ameliorate at least one symptom of a disorder being treated as compared to that symptom in the absence of use of the anti-IL-11 antibody or antigen-binding fragment thereof composition or antigen-binding fragment thereof, the result should be considered an effective treatment of the underlying disorder regardless of whether all the symptoms of the disorder are alleviated or not.

The term “package insert” is used to refer to instructions customarily included in commercial packages of therapeutic products, that contain information about the indications, usage, administration, contraindications and/or warnings concerning the use of such therapeutic products.

### Anti-IL-11 Antibodies

Described and disclosed herein are anti-IL-11 antibodies, in particular human anti-IL-11 antibodies, as well as compositions and articles of manufacture comprising anti-IL-11 antibodies of the present disclosure. Also described are antigen-binding fragments of an anti-IL-11 antibody. An anti-IL-11 antibody and an antigen-binding fragment thereof each include at least a portion that binds (e.g., specifically binds) to IL-11, or a fragment thereof, preferably human IL-11. The generation of anti-IL-11 antibodies and their characterization is described in the Examples.

In some embodiments, the anti-IL-11 antibodies or antigen-binding fragments thereof comprise or consist of a VH having a set of CDRs (HCDR1, HCDR2, and HCDR3) comprising or consisting of the amino acid sequences disclosed in Table 1. The anti-IL-11 antibodies or antigen-binding fragments thereof may alternatively comprise a set of HCDRs corresponding to those HCDRs in one or more of the anti-IL-11 antibody VHs described in Table 2 (e.g., the HCDRs of Antibody B).

In some embodiments, the anti-IL-11 antibodies comprise or consist of a VL having a set of CDRs (LCDR1, LCDR2, and LCDR3) comprising or consisting of the amino acid sequences disclosed in Table 1. The anti-IL-11 antibodies or antigen-binding fragments thereof may alternatively comprise a set of LCDRs corresponding to those LCDRs in one or more of the anti-IL-11 antibody VLs described in Table 3 (e.g., the LCDRs of Antibody B).

In some embodiments, the anti-IL-11 antibodies or antibody fragments thereof comprise a VH having a set of CDRs (HCDR1, HCDR2, and HCDR3) as disclosed in Table 1, and a VL having a set of CDRs (LCDR1, LCDR2, and LCDR3) as disclosed in Table 1.

TABLE 1
KABAT NOMENCLATURE

| | Antibodies A-O | | |
|---|---|---|---|
| | KABAT SEQ | SEQ ID NO. | |
| HCDR1-1 | DYNMD | 1 | |
| HCDR1-2 | SYNMD | 2 | |
| HCDR2-1 | DINPHSGGPIYNQKFTG | 3 | |
| HCDR2-2 | DINPHNGGPIYNQKFTG | 4 | |
| HCDR2-3 | DINPHGGGPIYNQKFTG | 5 | |
| HCDR2-4 | DINPHQGGPIYNQKFTG | 6 | |
| HCDR3 | GELGHWYFDV | 7 | |
| LCDR1 | RASKSVSTSGYSYIH | 9 | |
| LCDR2-1 | LASNLES | 10 | |
| LCDR2-2 | LASNLDS | 11 | |
| LCDR2-3 | LASNLAS | 12 | |
| LCDR3 | QHSRDLPPT | 13 | |
| HCDR1- | X₁YNMD | 50 | wherein X₁ = D |
| Consensus | | | or S |
| HCDR2- | DINPHX₁GGPIYNQKFTG | 51 | wherein X₁ = S, |
| Consensus | | | N, G, or Q |
| LCDR2- | LASNLX₁S | 52 | wherein X₁ = E, |
| Consensus | | | D, or A |

In some embodiments, the anti-IL-11 antibody or antigen-binding fragment thereof comprises a VH comprising a set of complementarity-determining regions CDR1, CDR2, and CDR3 selected from the group consisting of:
(i) CDR1: SEQ ID NO: 1, CDR2: SEQ ID NO: 4, CDR3: SEQ ID NO: 7;
(ii) CDR1: SEQ ID NO: 2, CDR2: SEQ ID NO: 4, CDR3: SEQ ID NO: 7;
(iii) CDR1: SEQ ID NO: 1, CDR2: SEQ ID NO: 5, CDR3: SEQ ID NO: 7;
(iv) CDR1: SEQ ID NO: 1, CDR2: SEQ ID NO: 6, CDR3: SEQ ID NO: 7; or
(v) CDR1: SEQ ID NO: 1, CDR2: SEQ ID NO: 3, CDR3: SEQ ID NO: 7.

In some embodiments, the anti-IL-11 antibody or antigen-binding fragment thereof comprises a VL comprising a set of complementarity-determining regions CDR1, CDR2, and CDR3 selected from the group consisting of:
(i) CDR1: SEQ ID NO: 9, CDR2: SEQ ID NO: 10, CDR3: SEQ ID NO: 13;
(ii) CDR1: SEQ ID NO: 9, CDR2: SEQ ID NO: 11, CDR3: SEQ ID NO: 13; or
(iii) CDR1: SEQ ID NO: 9, CDR2: SEQ ID NO: 12, CDR3: SEQ ID NO: 13.

In some embodiments, an anti-IL-11 antibody or antigen-binding fragment thereof comprises a combination of a VH and a VL each comprising a set of complementarity-determining regions CDR1, CDR2 and CDR3 selected from the group consisting of:
(i) VH: CDR1: SEQ ID NO: 1, CDR2: SEQ ID NO: 4, CDR3: SEQ ID NO: 7, VL: CDR1: SEQ ID NO: 9, CDR2: SEQ ID NO: 10, CDR3: SEQ ID NO: 13;
(ii) VH: CDR1: SEQ ID NO: 1, CDR2: SEQ ID NO: 4, CDR3: SEQ ID NO: 7, VL: CDR1: SEQ ID NO: 9, CDR2: SEQ ID NO: 12, CDR3: SEQ ID NO: 13;
(iii) VH: CDR1: SEQ ID NO: 2, CDR2: SEQ ID NO: 4, CDR3: SEQ ID NO: 7, VL: CDR1: SEQ ID NO: 9, CDR2: SEQ ID NO: 11, CDR3: SEQ ID NO: 13;
(iv) VH: CDR1: SEQ ID NO: 2, CDR2: SEQ ID NO: 4, CDR3: SEQ ID NO: 7, VL: CDR1: SEQ ID NO: 9, CDR2: SEQ ID NO: 10, CDR3: SEQ ID NO: 13;
(v) VH: CDR1: SEQ ID NO: 2, CDR2: SEQ ID NO: 4, CDR3: SEQ ID NO: 7, VL: CDR1: SEQ ID NO: 9, CDR2: SEQ ID NO: 12, CDR3: SEQ ID NO: 13;
(vi) VH: CDR1: SEQ ID NO: 1, CDR2: SEQ ID NO: 5, CDR3: SEQ ID NO: 7, VL: CDR1: SEQ ID NO: 9, CDR2: SEQ ID NO: 11, CDR3: SEQ ID NO: 13;
(vii) VH: CDR1: SEQ ID NO: 1, CDR2: SEQ ID NO: 5, CDR3: SEQ ID NO: 7, VL: CDR1: SEQ ID NO: 9, CDR2: SEQ ID NO: 10, CDR3: SEQ ID NO: 13;
(viii) VH: CDR1: SEQ ID NO: 1, CDR2: SEQ ID NO: 5, CDR3: SEQ ID NO: 7, VL: CDR1: SEQ ID NO: 9, CDR2: SEQ ID NO: 12, CDR3: SEQ ID NO: 13;
(ix) VH: CDR1: SEQ ID NO: 1, CDR2: SEQ ID NO: 6, CDR3: SEQ ID NO: 7, VL: CDR1: SEQ ID NO: 9, CDR2: SEQ ID NO: 11, CDR3: SEQ ID NO: 13;
(x) VH: CDR1: SEQ ID NO: 1, CDR2: SEQ ID NO: 6, CDR3: SEQ ID NO: 7, VL: CDR1: SEQ ID NO: 9, CDR2: SEQ ID NO: 10, CDR3: SEQ ID NO: 13;
(xi) VH: CDR1: SEQ ID NO: 1, CDR2: SEQ ID NO: 6, CDR3: SEQ ID NO: 7, VL: CDR1: SEQ ID NO: 9, CDR2: SEQ ID NO: 12, CDR3: SEQ ID NO: 13;
(xii) VH: CDR1: SEQ ID NO: 1, CDR2: SEQ ID NO: 3, CDR3: SEQ ID NO: 7, VL: CDR1: SEQ ID NO: 9, CDR2: SEQ ID NO: 11, CDR3: SEQ ID NO: 13;
(xiii) VH: CDR1: SEQ ID NO: 1, CDR2: SEQ ID NO: 3, CDR3: SEQ ID NO: 7, VL: CDR1: SEQ ID NO: 9, CDR2: SEQ ID NO: 10, CDR3: SEQ ID NO: 13; or
(xiv) VH: CDR1: SEQ ID NO: 1, CDR2: SEQ ID NO: 3, CDR3: SEQ ID NO: 7, VL: CDR1: SEQ ID NO: 9, CDR2: SEQ ID NO: 12, CDR3: SEQ ID NO: 13.

In some embodiments, the anti-IL-11 antibodies or antigen-binding fragments thereof comprises sets/combinations of complementarity determining region (CDR) sequences derived from the VH and/or VL domains of the anti-IL-11 antibodies or antigen-binding fragments disclosed herein.

Also provided by the present disclosure are anti-IL-11 antibodies or antigen-binding fragments thereof that comprise a heavy and/or a light chain variable region as set forth in Tables 2 and 3.

In some embodiments, the anti-IL-11 antibody or antigen-binding fragment thereof comprises or consists of a variable heavy chain sequence selected from the group consisting of SEQ ID NOs: 14, 15, 16, 17, and 18, and/or a variable light chain sequence selected from the group consisting of: SEQ ID NOs: 19, 20, and 21.

TABLE 2
Heavy Chain Variable Region (VH) Amino Acid Sequences

| Antibody | VH Sequence | SEQ ID NO. |
|---|---|---|
| Antibody A | EVQLVQSGAEVKKPGASVKISCKASGYTFTDYNMDW | 15 |
| Antibody B | VKQAPGQRLEWIGDINPHNGGPIYNQKFTGRATLTVD | |
| Antibody C | KSASTAYMELSSLRSEDTAVYYCARGELGHWYFDVW | |
| | GQGTTVTVSS | |
| Antibody D | EVQLVQSGAEVKKPGASVKISCKASGYTFTSYNMDW | 16 |
| Antibody E | VKQAPGQRLEWIGDINPHNGGPIYNQKFTGRATLTVD | |
| Antibody F | KSASTAYMELSSLRSEDTAVYYCARGELGHWYFDVW | |
| | GQGTTVTVSS | |
| Antibody G | EVQLVQSGAEVKKPGASVKISCKASGYTFTDYNMDW | 17 |
| Antibody H | VKQAPGQRLEWIGDINPHGGGPIYNQKFTGRATLTVD | |
| Antibody I | KSASTAYMELSSLRSEDTAVYYCARGELGHWYFDVW | |
| | GQGTTVTVSS | |
| Antibody J | EVQLVQSGAEVKKPGASVKISCKASGYTFTDYNMDW | 18 |
| Antibody K | VKQAPGQRLEWIGDINPHQGGPIYNQKFTGRATLTVD | |
| Antibody L | KSASTAYMELSSLRSEDTAVYYCARGELGHWYFDVW | |
| | GQGTTVTVSS | |
| Antibody M | EVQLVQSGAEVKKPGASVKISCKASGYTFTDYNMDW | 14 |
| Antibody N | VKQAPGQRLEWIGDINPHSGGPIYNQKFTGRATLTVD | |
| Antibody O | KSASTAYMELSSLRSEDTAVYYCARGELGHWYFDVW | |
| | GQGTTVTVSS | |

TABLE 3
Light Chain Variable Region Amino Acid Sequences

| Antibody | VL Sequence | SEQ ID NO. |
|---|---|---|
| Antibody A | DIVLTQSPASLALSPGERATLSCRASKSVSTSGYSYIH | 20 |
| Antibody D | WYQQKPGQAPRLLIYLASNLDSGVPARFSGSGSGTD | |
| Antibody G | FTLTISSLEEEDFATYYCQHSRDLPPTFGQGTKLEIK | |
| Antibody J | | |
| Antibody M | | |
| Antibody B | DIVLTQSPASLALSPGERATLSCRASKSVSTSGYSYIH | 19 |
| Antibody E | WYQQKPGQAPRLLIYLASNLESGVPARFSGSGSGTD | |
| Antibody H | FTLTISSLEEEDFATYYCQHSRDLPPTFGQGTKLEIK | |
| Antibody K | | |
| Antibody N | | |
| Antibody C | DIVLTQSPASLALSPGERATLSCRASKSVSTSGYSYIH | 21 |
| Antibody F | WYQQKPGQAPRLLIYLASNLASGVPARFSGSGSGTD | |
| Antibody I | FTLTISSLEEEDFATYYCQHSRDLPPTFGQGTKLEIK | |
| Antibody L | | |
| Antibody O | | |

The disclosure also provides anti-IL-11 antibodies and antigen-binding fragments thereof that comprise or consist of a combination of a light chain variable region and a heavy chain variable region as set forth in Table 4.

In some embodiments, the anti-IL-11 antibody or antigen-binding fragment thereof comprises or consists of:
a VH that has about 80%, about 85%, about 90%, about 95%, about 96%, about 97%, about 98%, about 99% or about 100% sequence identity to the amino acid sequence of SEQ ID NOs: 14, 15, 16, 17, or 18; and
a VL that has about 80%, about 85%, about 90%, about 95%, about 96%, about 97%, about 98%, about 99% or about 100% sequence identity to SEQ ID NOs: 19, 20, or 21.

In some embodiments, the anti-IL-11 antibody or antigen-binding fragment thereof comprises or consist of a pair of variable heavy chain and variable light chain sequences, selected from the following combinations:
(i) a variable heavy chain sequence that is 90%, 95%, or 99% identical to SEQ ID NO: 15, and a variable light chain sequence that is 90%, 95%, or 99% identical to SEQ ID NO: 19;
(ii) a variable heavy chain sequence that is 90%, 95%, or 99% identical to SEQ ID NO: 15, and a variable light chain sequence that is 90%, 95%, or 99% identical to SEQ ID NO: 21;
(iii) a variable heavy chain sequence that is 90%, 95%, or 99% identical to SEQ ID NO: 16, and a variable light chain sequence that is 90%, 95%, or 99% identical to SEQ ID NO: 20;
(iv) a variable heavy chain sequence that is 90%, 95%, or 99% identical to SEQ ID NO: 16, and a variable light chain sequence that is 90%, 95%, or 99% identical to SEQ ID NO: 19;
(v) a variable heavy chain sequence that is 90%, 95%, or 99% identical to SEQ ID NO: 16, and a variable light chain sequence that is 90%, 95%, or 99% identical to SEQ ID NO: 21;
(vi) a variable heavy chain sequence that is 90%, 95%, or 99% identical to SEQ ID NO: 17, and a variable light chain sequence that is 90%, 95%, or 99% identical to SEQ ID NO: 20;
(vii) a variable heavy chain sequence that is 90%, 95%, or 99% identical to SEQ ID NO: 17, and a variable light chain sequence that is 90%, 95%, or 99% identical to SEQ ID NO: 19;
(viii) a variable heavy chain sequence that is 90%, 95%, or 99% identical to SEQ ID NO: 17, and a variable light chain sequence that is 90%, 95%, or 99% identical to SEQ ID NO: 21;
(ix) a variable heavy chain sequence that is 90%, 95%, or 99% identical to SEQ ID NO: 18, and a variable light chain sequence that is 90%, 95%, or 99% identical to SEQ ID NO: 20;
(x) a variable heavy chain sequence that is 90%, 95%, or 99% identical to SEQ ID NO: 18, and a variable light chain sequence that is 90%, 95%, or 99% identical to SEQ ID NO: 19;
(xi) a variable heavy chain sequence that is 90%, 95%, or 99% identical to SEQ ID NO: 18, and a variable light chain sequence that is 90%, 95%, or 99% identical to SEQ ID NO: 21;
(xii) a variable heavy chain sequence that is 90%, 95%, or 99% identical to SEQ ID NO: 14, and a variable light chain sequence that is 90%, 95%, or 99% identical to SEQ ID NO: 20;
(xiii) a variable heavy chain sequence that is 90%, 95%, or 99% identical to SEQ ID NO: 14, and a variable light chain sequence that is 90%, 95%, or 99% identical to SEQ ID NO: 19; or
(xiv) a variable heavy chain sequence that is 90%, 95%, or 99% identical to SEQ ID NO: 14, and a variable light chain sequence that is 90%, 95%, or 99% identical to SEQ ID NO: 21.

In some embodiments, the anti-IL-11 antibody or antigen-binding fragment thereof comprises or consists of a pair of variable heavy chain and variable light chain sequences, selected from the following combinations:
(i) a variable heavy chain sequence comprising SEQ ID NO: 15, and a variable light chain sequence comprising SEQ ID NO: 19;
(ii) a variable heavy chain sequence comprising SEQ ID NO: 15, and a variable light chain sequence comprising SEQ ID NO: 21;
(iii) a variable heavy chain sequence comprising SEQ ID NO: 16, and a variable light chain sequence comprising SEQ ID NO: 20;
(iv) a variable heavy chain sequence comprising SEQ ID NO: 16, and a variable light chain sequence comprising SEQ ID NO: 19;
(v) a variable heavy chain sequence comprising SEQ ID NO: 16, and a variable light chain sequence comprising SEQ ID NO: 21;
(vi) a variable heavy chain sequence comprising SEQ ID NO: 17, and a variable light chain sequence comprising SEQ ID NO: 20;
(vii) a variable heavy chain sequence comprising SEQ ID NO: 17, and a variable light chain sequence comprising SEQ ID NO: 19;
(viii) a variable heavy chain sequence comprising SEQ ID NO: 17, and a variable light chain sequence comprising SEQ ID NO: 21;
(ix) a variable heavy chain sequence comprising SEQ ID NO: 18, and a variable light chain sequence comprising SEQ ID NO: 20;
(x) a variable heavy chain sequence comprising SEQ ID NO: 18, and a variable light chain sequence comprising SEQ ID NO: 19;
(xi) a variable heavy chain sequence comprising SEQ ID NO: 18, and a variable light chain sequence comprising SEQ ID NO: 21;
(xii) a variable heavy chain sequence comprising SEQ ID NO: 14, and a variable light chain sequence comprising SEQ ID NO: 20;
(xiii) a variable heavy chain sequence comprising SEQ ID NO: 14, and a variable light chain sequence comprising SEQ ID NO: 19; or
(xiv) a variable heavy chain sequence comprising SEQ ID NO: 14, and a variable light chain sequence comprising SEQ ID NO: 21. Such combinations of VHs and VLs are provided in Table 4.

Also provided is an anti-IL-11 antibody or antigen-binding fragment thereof that comprises or consists of:
a VH that has about 80%, about 85%, about 90%, about 95%, about 96%, about 97%, about 98%, about 99% or about 100% sequence identity to the amino acid sequence of SEQ ID NOs: 14, 15, 16, 17, or 18; and a VL that has about 80%, about 85%, about 90%, about 95%, about 96%, about 97%, about 98%, about 99% or about 100% sequence identity to SEQ ID NOs: 19, 20, or 21, respectively;
wherein the VH comprises the amino acid sequence of SEQ ID NO: 1 or SEQ ID NO: 2 (H-CDR1), the amino acid sequence of SEQ ID NO: 3, SEQ ID NO: 4, SEQ ID NO: 5, or SEQ ID NO: 6 (H-CDR2), and the amino acid sequence of SEQ ID NO: 7 (H-CDR3); and
wherein the VL comprises the amino acid sequence of SEQ ID NO: 9 (L-CDR1), the amino acid sequence of SEQ ID NO: 10, SEQ ID NO: 11, or SEQ ID NO: 12 (L-CDR2), and the amino acid sequence of SEQ ID NO: 13 (L-CDR3).

TABLE 4
Heavy Chain Variable Region (VH) and Light Chain Variable Region (VL) Amino
Acid Sequences

| | | SEQ | | SEQ |
|---|---|---|---|---|
| Antibody | VH SEQUENCE | ID NO. | VL SEQUENCE | ID NO. |
| A | EVQLVQSGAEVKKPGASVKI | 15 | DIVLTQSPASLALSPGERATLS | 20 |
| | SCKASGYTFTDYNMDWVKQ | | CRASKSVSTSGYSYIHWYQQ | |
| | APGQRLEWIGDINPHNGGPI | | KPGQAPRLLIYLASNLDSGVP | |
| | YNQKFTGRATLTVDKSASTA | | ARFSGSGSGTDFTLTISSLEEE | |
| | YMELSSLRSEDTAVYYCARG | | DFATYYCQHSRDLPPTFGQG | |
| | ELGHWYFDVWGQGTTVTVS | | TKLEIK | |
| | S | | | |
| B | EVQLVQSGAEVKKPGASVKI | 15 | DIVLTQSPASLALSPGERATLS | 19 |
| | SCKASGYTFTDYNMDWVKQ | | CRASKSVSTSGYSYIHWYQQ | |
| | APGQRLEWIGDINPHNGGPI | | KPGQAPRLLIYLASNLESGVP | |
| | YNQKFTGRATLTVDKSASTA | | ARFSGSGSGTDFTLTISSLEEE | |
| | YMELSSLRSEDTAVYYCARG | | DFATYYCQHSRDLPPTFGQG | |
| | ELGHWYFDVWGQGTTVTVS | | TKLEIK | |
| | S | | | |
| C | EVQLVQSGAEVKKPGASVKI | 15 | DIVLTQSPASLALSPGERATLS | 21 |
| | SCKASGYTFTDYNMDWVKQ | | CRASKSVSTSGYSYIHWYQQ | |
| | APGQRLEWIGDINPHNGGPI | | KPGQAPRLLIYLASNLASGVP | |
| | YNQKFTGRATLTVDKSASTA | | ARFSGSGSGTDFTLTISSLEEE | |
| | YMELSSLRSEDTAVYYCARG | | DFATYYCQHSRDLPPTFGQG | |
| | ELGHWYFDVWGQGTTVTVS | | TKLEIK | |
| | S | | | |
| D | EVQLVQSGAEVKKPGASVKI | 16 | DIVLTQSPASLALSPGERATLS | 20 |
| | SCKASGYTFTSYNMDWVKQ | | CRASKSVSTSGYSYIHWYQQ | |
| | APGQRLEWIGDINPHNGGPI | | KPGQAPRLLIYLASNLDSGVP | |
| | YNQKFTGRATLTVDKSASTA | | ARFSGSGSGTDFTLTISSLEEE | |
| | YMELSSLRSEDTAVYYCARG | | DFATYYCQHSRDLPPTFGQG | |
| | ELGHWYFDVWGQGTTVTVS | | TKLEIK | |
| | S | | | |
| E | EVQLVQSGAEVKKPGASVKI | 16 | DIVLTQSPASLALSPGERATLS | 19 |
| | SCKASGYTFTSYNMDWVKQ | | CRASKSVSTSGYSYIHWYQQ | |
| | APGQRLEWIGDINPHNGGPI | | KPGQAPRLLIYLASNLESGVP | |
| | YNQKFTGRATLTVDKSASTA | | ARFSGSGSGTDFTLTISSLEEE | |
| | YMELSSLRSEDTAVYYCARG | | DFATYYCQHSRDLPPTFGQG | |
| | ELGHWYFDVWGQGTTVTVS | | TKLEIK | |
| | S | | | |
| F | EVQLVQSGAEVKKPGASVKI | 16 | DIVLTQSPASLALSPGERATLS | 21 |
| | SCKASGYTFTSYNMDWVKQ | | CRASKSVSTSGYSYIHWYQQ | |
| | APGQRLEWIGDINPHNGGPI | | KPGQAPRLLIYLASNLASGVP | |
| | YNQKFTGRATLTVDKSASTA | | ARFSGSGSGTDFTLTISSLEEE | |
| | YMELSSLRSEDTAVYYCARG | | DFATYYCQHSRDLPPTFGQG | |
| | ELGHWYFDVWGQGTTVTVS | | TKLEIK | |
| | S | | | |
| G | EVQLVQSGAEVKKPGASVKI | 17 | DIVLTQSPASLALSPGERATLS | 20 |
| | SCKASGYTFTDYNMDWVKQ | | CRASKSVSTSGYSYIHWYQQ | |
| | APGQRLEWIGDINPHGGGPI | | KPGQAPRLLIYLASNLDSGVP | |
| | YNQKFTGRATLTVDKSASTA | | ARFSGSGSGTDFTLTISSLEEE | |
| | YMELSSLRSEDTAVYYCARG | | DFATYYCQHSRDLPPTFGQG | |
| | ELGHWYFDVWGQGTTVTVS | | TKLEIK | |
| | S | | | |
| H | EVQLVQSGAEVKKPGASVKI | 17 | DIVLTQSPASLALSPGERATLS | 19 |
| | SCKASGYTFTDYNMDWVKQ | | CRASKSVSTSGYSYIHWYQQ | |
| | APGQRLEWIGDINPHGGGPI | | KPGQAPRLLIYLASNLESGVP | |
| | YNQKFTGRATLTVDKSASTA | | ARFSGSGSGTDFTLTISSLEEE | |
| | YMELSSLRSEDTAVYYCARG | | DFATYYCQHSRDLPPTFGQG | |
| | ELGHWYFDVWGQGTTVTVS | | TKLEIK | |
| | S | | | |
| I | EVQLVQSGAEVKKPGASVKI | 17 | DIVLTQSPASLALSPGERATLS | 21 |
| | SCKASGYTFTDYNMDWVKQ | | CRASKSVSTSGYSYIHWYQQ | |
| | APGQRLEWIGDINPHGGGPI | | KPGQAPRLLIYLASNLASGVP | |
| | YNQKFTGRATLTVDKSASTA | | ARFSGSGSGTDFTLTISSLEEE | |
| | YMELSSLRSEDTAVYYCARG | | DFATYYCQHSRDLPPTFGQG | |
| | ELGHWYFDVWGQGTTVTVS | | TKLEIK | |
| | S | | | |
| J | EVQLVQSGAEVKKPGASVKI | 18 | DIVLTQSPASLALSPGERATLS | 20 |
| | SCKASGYTFTDYNMDWVKQ | | CRASKSVSTSGYSYIHWYQQ | |
| | APGQRLEWIGDINPHQGGPI | | KPGQAPRLLIYLASNLDSGVP | |
| | YNQKFTGRATLTVDKSASTA | | ARFSGSGSGTDFTLTISSLEEE | |
| | YMELSSLRSEDTAVYYCARG | | DFATYYCQHSRDLPPTFGQG | |
| | ELGHWYFDVWGQGTTVTVS | | TKLEIK | |
| | S | | | |
| K | EVQLVQSGAEVKKPGASVKI | 18 | DIVLTQSPASLALSPGERATLS | 19 |
| | SCKASGYTFTDYNMDWVKQ | | CRASKSVSTSGYSYIHWYQQ | |
| | APGQRLEWIGDINPHQGGPI | | KPGQAPRLLIYLASNLESGVP | |
| | YNQKFTGRATLTVDKSASTA | | ARFSGSGSGTDFTLTISSLEEE | |
| | YMELSSLRSEDTAVYYCARG | | DFATYYCQHSRDLPPTFGQG | |
| | ELGHWYFDVWGQGTTVTVS | | TKLEIK | |
| | S | | | |
| L | EVQLVQSGAEVKKPGASVKI | 18 | DIVLTQSPASLALSPGERATLS | 21 |
| | SCKASGYTFTDYNMDWVKQ | | CRASKSVSTSGYSYIHWYQQ | |
| | APGQRLEWIGDINPHQGGPI | | KPGQAPRLLIYLASNLASGVP | |
| | YNQKFTGRATLTVDKSASTA | | ARFSGSGSGTDFTLTISSLEEE | |
| | YMELSSLRSEDTAVYYCARG | | DFATYYCQHSRDLPPTFGQG | |
| | ELGHWYFDVWGQGTTVTVS | | TKLEIK | |
| | S | | | |
| M | EVQLVQSGAEVKKPGASVKI | 14 | DIVLTQSPASLALSPGERATLS | 20 |
| | SCKASGYTFTDYNMDWVKQ | | CRASKSVSTSGYSYIHWYQQ | |
| | APGQRLEWIGDINPHSGGPI | | KPGQAPRLLIYLASNLDSGVP | |
| | YNQKFTGRATLTVDKSASTA | | ARFSGSGSGTDFTLTISSLEEE | |
| | YMELSSLRSEDTAVYYCARG | | DFATYYCQHSRDLPPTFGQG | |
| | ELGHWYFDVWGQGTTVTVS | | TKLEIK | |
| | S | | | |
| N | EVQLVQSGAEVKKPGASVKI | 14 | DIVLTQSPASLALSPGERATLS | 19 |
| | SCKASGYTFTDYNMDWVKQ | | CRASKSVSTSGYSYIHWYQQ | |
| | APGQRLEWIGDINPHSGGPI | | KPGQAPRLLIYLASNLESGVP | |
| | YNQKFTGRATLTVDKSASTA | | ARFSGSGSGTDFTLTISSLEEE | |
| | YMELSSLRSEDTAVYYCARG | | DFATYYCQHSRDLPPTFGQG | |
| | ELGHWYFDVWGQGTTVTVS | | TKLEIK | |
| | S | | | |
| O | EVQLVQSGAEVKKPGASVKI | 14 | DIVLTQSPASLALSPGERATLS | 21 |
| | SCKASGYTFTDYNMDWVKQ | | CRASKSVSTSGYSYIHWYQQ | |
| | APGQRLEWIGDINPHSGGPI | | KPGQAPRLLIYLASNLASGVP | |
| | YNQKFTGRATLTVDKSASTA | | ARFSGSGSGTDFTLTISSLEEE | |
| | YMELSSLRSEDTAVYYCARG | | DFATYYCQHSRDLPPTFGQG | |
| | ELGHWYFDVWGQGTTVTVS | | TKLEIK | |
| | S | | | |

In some embodiments, a variable region of an anti-IL-11 antibody disclosed herein may be covalently attached at a C-terminal amino acid to at least one other antibody domain or a fragment thereof. Thus, for example, a VH domain that is present in the variable region domain may be linked to an immunoglobulin CH1 domain, or a fragment thereof. Similarly, a VL domain may be linked to a CK domain or a fragment thereof. In this way, for example, the antibody may be a Fab fragment wherein the antigen-binding domain contains associated VH and VL domains covalently linked at their C-termini to a CH1 and CK domain, respectively. The CH1 domain may be extended with further amino acids, for example, to provide a hinge region or a portion of a hinge region domain as found in a Fab fragment, or to provide further domains, such as antibody CH2 and CH3 domains. For example, anti-IL-11 antibodies of the present disclosure may comprise a heavy and/or light chain constant region as set forth in Table 5 below.

TABLE 5
Exemplary Heavy Chain and Light Chain Constant Region Sequences

| Antibody | Sequence | SEQ ID NO: |
|---|---|---|
| IgG1-HC | ASTKGPSVFPLAPSSKSTSGGTAALGCLVKDYFPEPVT | 42 |
| | VSWNSGALTSGVHTFPAVLQSSGLYSLSSVVTVPSSSL | |
| | GTQTYICNVNHKPSNTKVDKRVEPKSCDKTHTCPPCP | |
| | APELLGGPSVFLFPPKPKDTLMISRTPEVTCVVVDVSH | |
| | EDPEVKFNWYVDGVEVHNAKTKPREEQYNSTYRVVS | |
| | VLTVLHQDWLNGKEYKCKVSNKALPAPIEKTISKAKGQ | |
| | PREPQVYTLPPSREEMTKNQVSLTCLVKGFYPSDIAVE | |
| | WESNGQPENNYKTTPPVLDSDGSFFLYSKLTVDKSRW | |
| | QQGNVFSCSVMHEALHNHYTQKSLSLSPGK | |
| IgG1-LC | RTVAAPSVFIFPPSDEQLKSGTASVVCLLNNFYPREAK | 43 |
| | VQWKVDNALQSGNSQESVTEQDSKDSTYSLSSTLTLS | |
| | KADYEKHKVYACEVTHQGLSSPVTKSFNRGEC | |
| IgG4-HC | ASTKGPSVFPLAPCSRSTSESTAALGCLVKDYFPEPVT | 44 |
| | VSWNSGALTSGVHTFPAVLQSSGLYSLSSVVTVPSSSL | |
| | GTKTYTCNVDHKPSNTKVDKRVESKYGPPCPPCPAPE | |
| | FLGGPSVFLFPPKPKDTLMISRTPEVTCVVVDVSQEDP | |
| | EVQFNWYVDGVEVHNAKTKPREEQFNSTYRVVSVLTV | |
| | LHQDWLNGKEYKCKVSNKGLPSSIEKTISKAKGQPREP | |
| | QVYTLPPSQEEMTKNQVSLTCLVKGFYPSDIAVEWES | |
| | NGQPENNYKTTPPVLDSDGSFFLYSRLTVDKSRWQEG | |
| | NVFSCSVMHEALHNHYTQKSLSLSLGK | |
| IgG4-LC | RTVAAPSVFIFPPSDEQLKSGTASVVCLLNNFYPREAK | 45 |
| | VQWKVDNALQSGNSQESVTEQDSKDSTYSLSSTLTLS | |
| | KADYEKHKVYACEVTHQGLSSPVTKSFNRGEC | |
| IgG1-KO-HC | ASTKGPSVFPLAPSSKSTSGGTAALGCLVKDYFPEPVT | 46 |
| | VSWNSGALTSGVHTFPAVLQSSGLYSLSSVVTVPSSSL | |
| | GTQTYICNVNHKPSNTKVDKRVEPKSCDKTHTCPPCP | |
| | APEAAGGPSVFLFPPKPKDTLMISRTPEVTCVVVDVSH | |
| | EDPEVKFNWYVDGVEVHNAKTKPREEQYNSTYRVVS | |
| | VLTVLHQDWLNGKEYKCKVSNKALPAPIEKTISKAKGQ | |
| | PREPQVYTLPPSREEMTKNQVSLTCLVKGFYPSDIAVE | |
| | WESNGQPENNYKTTPPVLDSDGSFFLYSKLTVDKSRW | |
| | QQGNVFSCSVMHEALHNHYTQKSLSLSPGK | |
| IgG1-KO-LC | RTVAAPSVFIFPPSDEQLKSGTASVVCLLNNFYPREAK | 47 |
| | VQWKVDNALQSGNSQESVTEQDSKDSTYSLSSTLTLS | |
| | KADYEKHKVYACEVTHQGLSSPVTKSFNRGEC | |

In some aspects, the anti-IL-11 antibodies provided herein comprise a pair of the variable heavy chain and variable light chain sequences in combination with a wild-type human IgG1 heavy chain constant region (CH) and wild-type human kappa light chain (CL) region. For example, and not for the purpose of limitation, the disclosure provides anti-IL-11 antibodies comprising a heavy chain variable region (VH) of the disclosure in combination with a wild-type human IgG1 constant heavy chain (CH) immunoglobulin sequence selected from the sequences set forth in SEQ ID NOs: 42 or 46, and a wild-type human immunoglobulin light chain having a light chain variable region of the invention (VL) in combination with an immunoglobulin light chain constant region (CL). In some aspects, the CL region comprises a human kappa light chain region comprising the sequence set forth in SEQ ID NO: 43, 45, or 47.

In some embodiments, the anti-IL-11 antibody comprises one or more point mutations (e.g., amino acid substitutions and/or deletions) at targeted sites in the Fc region to enhance or decrease ADCC, and/or ADCP and/or CDC activities relative to the activity of the same antibody (e.g., same variable VH and VL regions or set of 6 set CDR sequences) comprising a wild-type IgG1 CH sequence (e.g., a human IgG1 CH sequence). One of skill in the art will readily appreciate that this aspect of the disclosure may be a chimeric or humanized anti-IL-11 antibody comprising variable regions (i.e., VH and VL) regions selected from the pairs of antibody sequences (VH and VL) in Table 4. Alternatively, the Fc-engineered anti-anti-IL-11 antibody comprising point mutations can be a fully human sequence comprising variable regions selected from the pairs of antibody sequences (VH and VL) or the sets of VH and VL CDRs in Tables 2-4.

Additionally, the disclosure provides anti-IL-11 antibodies and antigen-binding fragments thereof that comprise a heavy and a light chain as set forth in Table 6 below.

TABLE 6
Heavy and Light Chain Sequences of Anti-IL-11 Antibodies

| | | SEQ | | SEQ |
|---|---|---|---|---|
| Antibody | HC Sequence | ID NO. | LC Sequence | ID NO. |
| Antibody A | EVQLVQSGAEVKKPGASVKIS | 27 | DIVLTQSPASLALSPGER | 32 |
| | CKASGYTFTDYNMDWVKQAP | | ATLSCRASKSVSTSGYSY | |
| | GQRLEWIGDINPHNGGPIYNQ | | IHWYQQKPGQAPRLLIYL | |
| | KFTGRATLTVDKSASTAYMELS | | ASNLDSGVPARFSGSGS | |
| | SLRSEDTAVYYCARGELGHWY | | GTDFTLTISSLEEEDFATY | |
| | FDVWGQGTTVTVSSASTKGPS | | YCQHSRDLPPTFGQGTK | |
| | VFPLAPSSKSTSGGTAALGCLV | | LEIKRTVAAPSVFIFPPSD | |
| | KDYFPEPVTVSWNSGALTSGV | | EQLKSGTASVVCLLNNFY | |
| | HTFPAVLQSSGLYSLSSVVTVP | | PREAKVQWKVDNALQSG | |
| | SSSLGTQTYICNVNHKPSNTKV | | NSQESVTEQDSKDSTYS | |
| | DKRVEPKSCDKTHTCPPCPAP | | LSSTLTLSKADYEKHKVY | |
| | ELLGGPSVFLFPPKPKDTLMIS | | ACEVTHQGLSSPVTKSF | |
| | RTPEVTCVVVDVSHEDPEVKF | | NRGEC | |
| | NWYVDGVEVHNAKTKPREEQ | | | |
| | YNSTYRVVSVLTVLHQDWLNG | | | |
| | KEYKCKVSNKALPAPIEKTISKA | | | |
| | KGQPREPQVYTLPPSREEMTK | | | |
| | NQVSLTCLVKGFYPSDIAVEW | | | |
| | ESNGQPENNYKTTPPVLDSDG | | | |
| | SFFLYSKLTVDKSRWQQGNVF | | | |
| | SCSVMHEALHNHYTQKSLSLS | | | |
| | PG | | | |
| Antibody B | EVQLVQSGAEVKKPGASVKIS | 27 | DIVLTQSPASLALSPGER | 31 |
| | CKASGYTFTDYNMDWVKQAP | | ATLSCRASKSVSTSGYSY | |
| | GQRLEWIGDINPHNGGPIYNQ | | IHWYQQKPGQAPRLLIYL | |
| | KFTGRATLTVDKSASTAYMELS | | ASNLESGVPARFSGSGS | |
| | SLRSEDTAVYYCARGELGHWY | | GTDFTLTISSLEEEDFATY | |
| | FDVWGQGTTVTVSSASTKGPS | | YCQHSRDLPPTFGQGTK | |
| | VFPLAPSSKSTSGGTAALGCLV | | LEIKRTVAAPSVFIFPPSD | |
| | KDYFPEPVTVSWNSGALTSGV | | EQLKSGTASVVCLLNNFY | |
| | HTFPAVLQSSGLYSLSSVVTVP | | PREAKVQWKVDNALQSG | |
| | SSSLGTQTYICNVNHKPSNTKV | | NSQESVTEQDSKDSTYS | |
| | DKRVEPKSCDKTHTCPPCPAP | | LSSTLTLSKADYEKHKVY | |
| | ELLGGPSVFLFPPKPKDTLMIS | | ACEVTHQGLSSPVTKSF | |
| | RTPEVTCVVVDVSHEDPEVKF | | NRGEC | |
| | NWYVDGVEVHNAKTKPREEQ | | | |
| | YNSTYRVVSVLTVLHQDWLNG | | | |
| | KEYKCKVSNKALPAPIEKTISKA | | | |
| | KGQPREPQVYTLPPSREEMTK | | | |
| | NQVSLTCLVKGFYPSDIAVEW | | | |
| | ESNGQPENNYKTTPPVLDSDG | | | |
| | SFFLYSKLTVDKSRWQQGNVF | | | |
| | SCSVMHEALHNHYTQKSLSLS | | | |
| | PG | | | |
| Antibody C | EVQLVQSGAEVKKPGASVKIS | 27 | DIVLTQSPASLALSPGER | 33 |
| | CKASGYTFTDYNMDWVKQAP | | ATLSCRASKSVSTSGYSY | |
| | GQRLEWIGDINPHNGGPIYNQ | | IHWYQQKPGQAPRLLIYL | |
| | KFTGRATLTVDKSASTAYMELS | | ASNLDSGVPARFSGSGS | |
| | SLRSEDTAVYYCARGELGHWY | | GTDFTLTISSLEEEDFATY | |
| | FDVWGQGTTVTVSSASTKGPS | | YCQHSRDLPPTFGQGTK | |
| | VFPLAPSSKSTSGGTAALGCLV | | LEIKRTVAAPSVFIFPPSD | |
| | KDYFPEPVTVSWNSGALTSGV | | EQLKSGTASVVCLLNNFY | |
| | HTFPAVLQSSGLYSLSSVVTVP | | PREAKVQWKVDNALQSG | |
| | SSSLGTQTYICNVNHKPSNTKV | | NSQESVTEQDSKDSTYS | |
| | DKRVEPKSCDKTHTCPPCPAP | | LSSTLTLSKADYEKHKVY | |
| | ELLGGPSVFLFPPKPKDTLMIS | | ACEVTHQGLSSPVTKSF | |
| | RTPEVTCVVVDVSHEDPEVKF | | NRGEC | |
| | NWYVDGVEVHNAKTKPREEQ | | | |
| | YNSTYRVVSVLTVLHQDWLNG | | | |
| | KEYKCKVSNKALPAPIEKTISKA | | | |
| | KGQPREPQVYTLPPSREEMTK | | | |
| | NQVSLTCLVKGFYPSDIAVEW | | | |
| | ESNGQPENNYKTTPPVLDSDG | | | |
| | SFFLYSKLTVDKSRWQQGNVF | | | |
| | SCSVMHEALHNHYTQKSLSLS | | | |
| | PG | | | |
| Antibody D | EVQLVQSGAEVKKPGASVKIS | 28 | DIVLTQSPASLALSPGER | 32 |
| | CKASGYTFTSYNMDWVKQAP | | ATLSCRASKSVSTSGYSY | |
| | GQRLEWIGDINPHNGGPIYNQ | | IHWYQQKPGQAPRLLIYL | |
| | KFTGRATLTVDKSASTAYMELS | | ASNLDSGVPARFSGSGS | |
| | SLRSEDTAVYYCARGELGHWY | | GTDFTLTISSLEEEDFATY | |
| | FDVWGQGTTVTVSSASTKGPS | | YCQHSRDLPPTFGQGTK | |
| | VFPLAPSSKSTSGGTAALGCLV | | LEIKRTVAAPSVFIFPPSD | |
| | KDYFPEPVTVSWNSGALTSGV | | EQLKSGTASVVCLLNNFY | |
| | HTFPAVLQSSGLYSLSSVVTVP | | PREAKVQWKVDNALQSG | |
| | SSSLGTQTYICNVNHKPSNTKV | | NSQESVTEQDSKDSTYS | |
| | DKRVEPKSCDKTHTCPPCPAP | | LSSTLTLSKADYEKHKVY | |
| | ELLGGPSVFLFPPKPKDTLMIS | | ACEVTHQGLSSPVTKSF | |
| | RTPEVTCVVVDVSHEDPEVKF | | NRGEC | |
| | NWYVDGVEVHNAKTKPREEQ | | | |
| | YNSTYRVVSVLTVLHQDWLNG | | | |
| | KEYKCKVSNKALPAPIEKTISKA | | | |
| | KGQPREPQVYTLPPSREEMTK | | | |
| | NQVSLTCLVKGFYPSDIAVEW | | | |
| | ESNGQPENNYKTTPPVLDSDG | | | |
| | SFFLYSKLTVDKSRWQQGNVF | | | |
| | SCSVMHEALHNHYTQKSLSLS | | | |
| | PG | | | |
| Antibody E | EVQLVQSGAEVKKPGASVKIS | 28 | DIVLTQSPASLALSPGER | 31 |
| | CKASGYTFTSYNMDWVKQAP | | ATLSCRASKSVSTSGYSY | |
| | GQRLEWIGDINPHNGGPIYNQ | | IHWYQQKPGQAPRLLIYL | |
| | KFTGRATLTVDKSASTAYMELS | | ASNLESGVPARFSGSGS | |
| | SLRSEDTAVYYCARGELGHWY | | GTDFTLTISSLEEEDFATY | |
| | FDVWGQGTTVTVSSASTKGPS | | YCQHSRDLPPTFGQGTK | |
| | VFPLAPSSKSTSGGTAALGCLV | | LEIKRTVAAPSVFIFPPSD | |
| | KDYFPEPVTVSWNSGALTSGV | | EQLKSGTASVVCLLNNFY | |
| | HTFPAVLQSSGLYSLSSVVTVP | | PREAKVQWKVDNALQSG | |
| | SSSLGTQTYICNVNHKPSNTKV | | NSQESVTEQDSKDSTYS | |
| | DKRVEPKSCDKTHTCPPCPAP | | LSSTLTLSKADYEKHKVY | |
| | ELLGGPSVFLFPPKPKDTLMIS | | ACEVTHQGLSSPVTKSF | |
| | RTPEVTCVVVDVSHEDPEVKF | | NRGEC | |
| | NWYVDGVEVHNAKTKPREEQ | | | |
| | YNSTYRVVSVLTVLHQDWLNG | | | |
| | KEYKCKVSNKALPAPIEKTISKA | | | |
| | KGQPREPQVYTLPPSREEMTK | | | |
| | NQVSLTCLVKGFYPSDIAVEW | | | |
| | ESNGQPENNYKTTPPVLDSDG | | | |
| | SFFLYSKLTVDKSRWQQGNVF | | | |
| | SCSVMHEALHNHYTQKSLSLS | | | |
| | PG | | | |
| Antibody F | EVQLVQSGAEVKKPGASVKIS | 28 | DIVLTQSPASLALSPGER | 33 |
| | CKASGYTFTSYNMDWVKQAP | | ATLSCRASKSVSTSGYSY | |
| | GQRLEWIGDINPHNGGPIYNQ | | IHWYQQKPGQAPRLLIYL | |
| | KFTGRATLTVDKSASTAYMELS | | ASNLDSGVPARFSGSGS | |
| | SLRSEDTAVYYCARGELGHWY | | GTDFTLTISSLEEEDFATY | |
| | FDVWGQGTTVTVSSASTKGPS | | YCQHSRDLPPTFGQGTK | |
| | VFPLAPSSKSTSGGTAALGCLV | | LEIKRTVAAPSVFIFPPSD | |
| | KDYFPEPVTVSWNSGALTSGV | | EQLKSGTASVVCLLNNFY | |
| | HTFPAVLQSSGLYSLSSVVTVP | | PREAKVQWKVDNALQSG | |
| | SSSLGTQTYICNVNHKPSNTKV | | NSQESVTEQDSKDSTYS | |
| | DKRVEPKSCDKTHTCPPCPAP | | LSSTLTLSKADYEKHKVY | |
| | ELLGGPSVFLFPPKPKDTLMIS | | ACEVTHQGLSSPVTKSF | |
| | RTPEVTCVVVDVSHEDPEVKF | | NRGEC | |
| | NWYVDGVEVHNAKTKPREEQ | | | |
| | YNSTYRVVSVLTVLHQDWLNG | | | |
| | KEYKCKVSNKALPAPIEKTISKA | | | |
| | KGQPREPQVYTLPPSREEMTK | | | |
| | NQVSLTCLVKGFYPSDIAVEW | | | |
| | ESNGQPENNYKTTPPVLDSDG | | | |
| | SFFLYSKLTVDKSRWQQGNVF | | | |
| | SCSVMHEALHNHYTQKSLSLS | | | |
| | PG | | | |
| Antibody G | EVQLVQSGAEVKKPGASVKIS | 29 | DIVLTQSPASLALSPGER | 32 |
| | CKASGYTFTDYNMDWVKQAP | | ATLSCRASKSVSTSGYSY | |
| | GQRLEWIGDINPHGGGPIYNQ | | IHWYQQKPGQAPRLLIYL | |
| | KFTGRATLTVDKSASTAYMELS | | ASNLDSGVPARFSGSGS | |
| | SLRSEDTAVYYCARGELGHWY | | GTDFTLTISSLEEEDFATY | |
| | FDVWGQGTTVTVSSASTKGPS | | YCQHSRDLPPTFGQGTK | |
| | VFPLAPSSKSTSGGTAALGCLV | | LEIKRTVAAPSVFIFPPSD | |
| | KDYFPEPVTVSWNSGALTSGV | | EQLKSGTASVVCLLNNFY | |
| | HTFPAVLQSSGLYSLSSVVTVP | | PREAKVQWKVDNALQSG | |
| | SSSLGTQTYICNVNHKPSNTKV | | NSQESVTEQDSKDSTYS | |
| | DKRVEPKSCDKTHTCPPCPAP | | LSSTLTLSKADYEKHKVY | |
| | ELLGGPSVFLFPPKPKDTLMIS | | ACEVTHQGLSSPVTKSF | |
| | RTPEVTCVVVDVSHEDPEVKF | | NRGEC | |
| | NWYVDGVEVHNAKTKPREEQ | | | |
| | YNSTYRVVSVLTVLHQDWLNG | | | |
| | KEYKCKVSNKALPAPIEKTISKA | | | |
| | KGQPREPQVYTLPPSREEMTK | | | |
| | NQVSLTCLVKGFYPSDIAVEW | | | |
| | ESNGQPENNYKTTPPVLDSDG | | | |
| | SFFLYSKLTVDKSRWQQGNVF | | | |
| | SCSVMHEALHNHYTQKSLSLS | | | |
| | PG | | | |
| Antibody H | EVQLVQSGAEVKKPGASVKIS | 29 | DIVLTQSPASLALSPGER | 31 |
| | CKASGYTFTDYNMDWVKQAP | | ATLSCRASKSVSTSGYSY | |
| | GQRLEWIGDINPHGGGPIYNQ | | IHWYQQKPGQAPRLLIYL | |
| | KFTGRATLTVDKSASTAYMELS | | ASNLESGVPARFSGSGS | |
| | SLRSEDTAVYYCARGELGHWY | | GTDFTLTISSLEEEDFATY | |
| | FDVWGQGTTVTVSSASTKGPS | | YCQHSRDLPPTFGQGTK | |
| | VFPLAPSSKSTSGGTAALGCLV | | LEIKRTVAAPSVFIFPPSD | |
| | KDYFPEPVTVSWNSGALTSGV | | EQLKSGTASVVCLLNNFY | |
| | HTFPAVLQSSGLYSLSSVVTVP | | PREAKVQWKVDNALQSG | |
| | SSSLGTQTYICNVNHKPSNTKV | | NSQESVTEQDSKDSTYS | |
| | DKRVEPKSCDKTHTCPPCPAP | | LSSTLTLSKADYEKHKVY | |
| | ELLGGPSVFLFPPKPKDTLMIS | | ACEVTHQGLSSPVTKSF | |
| | RTPEVTCVVVDVSHEDPEVKF | | NRGEC | |
| | NWYVDGVEVHNAKTKPREEQ | | | |
| | YNSTYRVVSVLTVLHQDWLNG | | | |
| | KEYKCKVSNKALPAPIEKTISKA | | | |
| | KGQPREPQVYTLPPSREEMTK | | | |
| | NQVSLTCLVKGFYPSDIAVEW | | | |
| | ESNGQPENNYKTTPPVLDSDG | | | |
| | SFFLYSKLTVDKSRWQQGNVF | | | |
| | SCSVMHEALHNHYTQKSLSLS | | | |
| | PG | | | |
| Antibody I | EVQLVQSGAEVKKPGASVKIS | 29 | DIVLTQSPASLALSPGER | 33 |
| | CKASGYTFTDYNMDWVKQAP | | ATLSCRASKSVSTSGYSY | |
| | GQRLEWIGDINPHGGGPIYNQ | | IHWYQQKPGQAPRLLIYL | |
| | KFTGRATLTVDKSASTAYMELS | | ASNLDSGVPARFSGSGS | |
| | SLRSEDTAVYYCARGELGHWY | | GTDFTLTISSLEEEDFATY | |
| | FDVWGQGTTVTVSSASTKGPS | | YCQHSRDLPPTFGQGTK | |
| | VFPLAPSSKSTSGGTAALGCLV | | LEIKRTVAAPSVFIFPPSD | |
| | KDYFPEPVTVSWNSGALTSGV | | EQLKSGTASVVCLLNNFY | |
| | HTFPAVLQSSGLYSLSSVVTVP | | PREAKVQWKVDNALQSG | |
| | SSSLGTQTYICNVNHKPSNTKV | | NSQESVTEQDSKDSTYS | |
| | DKRVEPKSCDKTHTCPPCPAP | | LSSTLTLSKADYEKHKVY | |
| | ELLGGPSVFLFPPKPKDTLMIS | | ACEVTHQGLSSPVTKSF | |
| | RTPEVTCVVVDVSHEDPEVKF | | NRGEC | |
| | NWYVDGVEVHNAKTKPREEQ | | | |
| | YNSTYRVVSVLTVLHQDWLNG | | | |
| | KEYKCKVSNKALPAPIEKTISKA | | | |
| | KGQPREPQVYTLPPSREEMTK | | | |
| | NQVSLTCLVKGFYPSDIAVEW | | | |
| | ESNGQPENNYKTTPPVLDSDG | | | |
| | SFFLYSKLTVDKSRWQQGNVF | | | |
| | SCSVMHEALHNHYTQKSLSLS | | | |
| | PG | | | |
| Antibody J | EVQLVQSGAEVKKPGASVKIS | 30 | DIVLTQSPASLALSPGER | 32 |
| | CKASGYTFTDYNMDWVKQAP | | ATLSCRASKSVSTSGYSY | |
| | GQRLEWIGDINPHQGGPIYNQ | | IHWYQQKPGQAPRLLIYL | |
| | KFTGRATLTVDKSASTAYMELS | | ASNLDSGVPARFSGSGS | |
| | SLRSEDTAVYYCARGELGHWY | | GTDFTLTISSLEEEDFATY | |
| | FDVWGQGTTVTVSSASTKGPS | | YCQHSRDLPPTFGQGTK | |
| | VFPLAPSSKSTSGGTAALGCLV | | LEIKRTVAAPSVFIFPPSD | |
| | KDYFPEPVTVSWNSGALTSGV | | EQLKSGTASVVCLLNNFY | |
| | HTFPAVLQSSGLYSLSSVVTVP | | PREAKVQWKVDNALQSG | |
| | SSSLGTQTYICNVNHKPSNTKV | | NSQESVTEQDSKDSTYS | |
| | DKRVEPKSCDKTHTCPPCPAP | | LSSTLTLSKADYEKHKVY | |
| | ELLGGPSVFLFPPKPKDTLMIS | | ACEVTHQGLSSPVTKSF | |
| | RTPEVTCVVVDVSHEDPEVKF | | NRGEC | |
| | NWYVDGVEVHNAKTKPREEQ | | | |
| | YNSTYRVVSVLTVLHQDWLNG | | | |
| | KEYKCKVSNKALPAPIEKTISKA | | | |
| | KGQPREPQVYTLPPSREEMTK | | | |
| | NQVSLTCLVKGFYPSDIAVEW | | | |
| | ESNGQPENNYKTTPPVLDSDG | | | |
| | SFFLYSKLTVDKSRWQQGNVF | | | |
| | SCSVMHEALHNHYTQKSLSLS | | | |
| | PG | | | |
| Antibody K | EVQLVQSGAEVKKPGASVKIS | 30 | DIVLTQSPASLALSPGER | 31 |
| | CKASGYTFTDYNMDWVKQAP | | ATLSCRASKSVSTSGYSY | |
| | GQRLEWIGDINPHQGGPIYNQ | | IHWYQQKPGQAPRLLIYL | |
| | KFTGRATLTVDKSASTAYMELS | | ASNLESGVPARFSGSGS | |
| | SLRSEDTAVYYCARGELGHWY | | GTDFTLTISSLEEEDFATY | |
| | FDVWGQGTTVTVSSASTKGPS | | YCQHSRDLPPTFGQGTK | |
| | VFPLAPSSKSTSGGTAALGCLV | | LEIKRTVAAPSVFIFPPSD | |
| | KDYFPEPVTVSWNSGALTSGV | | EQLKSGTASVVCLLNNFY | |
| | HTFPAVLQSSGLYSLSSVVTVP | | PREAKVQWKVDNALQSG | |
| | SSSLGTQTYICNVNHKPSNTKV | | NSQESVTEQDSKDSTYS | |
| | DKRVEPKSCDKTHTCPPCPAP | | LSSTLTLSKADYEKHKVY | |
| | ELLGGPSVFLFPPKPKDTLMIS | | ACEVTHQGLSSPVTKSF | |
| | RTPEVTCVVVDVSHEDPEVKF | | NRGEC | |
| | NWYVDGVEVHNAKTKPREEQ | | | |
| | YNSTYRVVSVLTVLHQDWLNG | | | |
| | KEYKCKVSNKALPAPIEKTISKA | | | |
| | KGQPREPQVYTLPPSREEMTK | | | |
| | NQVSLTCLVKGFYPSDIAVEW | | | |
| | ESNGQPENNYKTTPPVLDSDG | | | |
| | SFFLYSKLTVDKSRWQQGNVF | | | |
| | SCSVMHEALHNHYTQKSLSLS | | | |
| | PG | | | |
| Antibody L | EVQLVQSGAEVKKPGASVKIS | 30 | DIVLTQSPASLALSPGER | 33 |
| | CKASGYTFTDYNMDWVKQAP | | ATLSCRASKSVSTSGYSY | |
| | GQRLEWIGDINPHQGGPIYNQ | | IHWYQQKPGQAPRLLIYL | |
| | KFTGRATLTVDKSASTAYMELS | | ASNLDSGVPARFSGSGS | |
| | SLRSEDTAVYYCARGELGHWY | | GTDFTLTISSLEEEDFATY | |
| | FDVWGQGTTVTVSSASTKGPS | | YCQHSRDLPPTFGQGTK | |
| | VFPLAPSSKSTSGGTAALGCLV | | LEIKRTVAAPSVFIFPPSD | |
| | KDYFPEPVTVSWNSGALTSGV | | EQLKSGTASWCLLNNFY | |
| | HTFPAVLQSSGLYSLSSVVTVP | | PREAKVQWKVDNALQSG | |
| | SSSLGTQTYICNVNHKPSNTKV | | NSQESVTEQDSKDSTYS | |
| | DKRVEPKSCDKTHTCPPCPAP | | LSSTLTLSKADYEKHKVY | |
| | ELLGGPSVFLFPPKPKDTLMIS | | ACEVTHQGLSSPVTKSF | |
| | RTPEVTCVVVDVSHEDPEVKF | | NRGEC | |
| | NWYVDGVEVHNAKTKPREEQ | | | |
| | YNSTYRVVSVLTVLHQDWLNG | | | |
| | KEYKCKVSNKALPAPIEKTISKA | | | |
| | KGQPREPQVYTLPPSREEMTK | | | |
| | NQVSLTCLVKGFYPSDIAVEW | | | |
| | ESNGQPENNYKTTPPVLDSDG | | | |
| | SFFLYSKLTVDKSRWQQGNVF | | | |
| | SCSVMHEALHNHYTQKSLSLS | | | |
| | PG | | | |
| Antibody M | EVQLVQSGAEVKKPGASVKIS | 26 | DIVLTQSPASLALSPGER | 32 |
| | CKASGYTFTDYNMDWVKQAP | | ATLSCRASKSVSTSGYSY | |
| | GQRLEWIGDINPHSGGPIYNQ | | IHWYQQKPGQAPRLLIYL | |
| | KFTGRATLTVDKSASTAYMELS | | ASNLDSGVPARFSGSGS | |
| | SLRSEDTAVYYCARGELGHWY | | GTDFTLTISSLEEEDFATY | |
| | FDVWGQGTTVTVSSASTKGPS | | YCQHSRDLPPTFGQGTK | |
| | VFPLAPSSKSTSGGTAALGCLV | | LEIKRTVAAPSVFIFPPSD | |
| | KDYFPEPVTVSWNSGALTSGV | | EQLKSGTASVVCLLNNFY | |
| | HTFPAVLQSSGLYSLSSVVTVP | | PREAKVQWKVDNALQSG | |
| | SSSLGTQTYICNVNHKPSNTKV | | NSQESVTEQDSKDSTYS | |
| | DKRVEPKSCDKTHTCPPCPAP | | LSSTLTLSKADYEKHKVY | |
| | ELLGGPSVFLFPPKPKDTLMIS | | ACEVTHQGLSSPVTKSF | |
| | RTPEVTCVVVDVSHEDPEVKF | | NRGEC | |
| | NWYVDGVEVHNAKTKPREEQ | | | |
| | YNSTYRVVSVLTVLHQDWLNG | | | |
| | KEYKCKVSNKALPAPIEKTISKA | | | |
| | KGQPREPQVYTLPPSREEMTK | | | |
| | NQVSLTCLVKGFYPSDIAVEW | | | |
| | ESNGQPENNYKTTPPVLDSDG | | | |
| | SFFLYSKLTVDKSRWQQGNVF | | | |
| | SCSVMHEALHNHYTQKSLSLS | | | |
| | PG | | | |
| Antibody N | EVQLVQSGAEVKKPGASVKIS | 26 | DIVLTQSPASLALSPGER | 31 |
| | CKASGYTFTDYNMDWVKQAP | | ATLSCRASKSVSTSGYSY | |
| | GQRLEWIGDINPHSGGPIYNQ | | IHWYQQKPGQAPRLLIYL | |
| | KFTGRATLTVDKSASTAYMELS | | ASNLESGVPARFSGSGS | |
| | SLRSEDTAVYYCARGELGHWY | | GTDFTLTISSLEEEDFATY | |
| | FDVWGQGTTVTVSSASTKGPS | | YCQHSRDLPPTFGQGTK | |
| | VFPLAPSSKSTSGGTAALGCLV | | LEIKRTVAAPSVFIFPPSD | |
| | KDYFPEPVTVSWNSGALTSGV | | EQLKSGTASVVCLLNNFY | |
| | HTFPAVLQSSGLYSLSSVVTVP | | PREAKVQWKVDNALQSG | |
| | SSSLGTQTYICNVNHKPSNTKV | | NSQESVTEQDSKDSTYS | |
| | DKRVEPKSCDKTHTCPPCPAP | | LSSTLTLSKADYEKHKVY | |
| | ELLGGPSVFLFPPKPKDTLMIS | | ACEVTHQGLSSPVTKSF | |
| | RTPEVTCVVVDVSHEDPEVKF | | NRGEC | |
| | NWYVDGVEVHNAKTKPREEQ | | | |
| | YNSTYRVVSVLTVLHQDWLNG | | | |
| | KEYKCKVSNKALPAPIEKTISKA | | | |
| | KGQPREPQVYTLPPSREEMTK | | | |
| | NQVSLTCLVKGFYPSDIAVEW | | | |
| | ESNGQPENNYKTTPPVLDSDG | | | |
| | SFFLYSKLTVDKSRWQQGNVF | | | |
| | SCSVMHEALHNHYTQKSLSLS | | | |
| | PG | | | |
| Antibody O | EVQLVQSGAEVKKPGASVKIS | 26 | DIVLTQSPASLALSPGER | 33 |
| | CKASGYTFTDYNMDWVKQAP | | ATLSCRASKSVSTSGYSY | |
| | GQRLEWIGDINPHSGGPIYNQ | | IHWYQQKPGQAPRLLIYL | |
| | KFTGRATLTVDKSASTAYMELS | | ASNLDSGVPARFSGSGS | |
| | SLRSEDTAVYYCARGELGHWY | | GTDFTLTISSLEEEDFATY | |
| | FDVWGQGTTVTVSSASTKGPS | | YCQHSRDLPPTFGQGTK | |
| | VFPLAPSSKSTSGGTAALGCLV | | LEIKRTVAAPSVFIFPPSD | |
| | KDYFPEPVTVSWNSGALTSGV | | EQLKSGTASVVCLLNNFY | |
| | HTFPAVLQSSGLYSLSSVVTVP | | PREAKVQWKVDNALQSG | |
| | SSSLGTQTYICNVNHKPSNTKV | | NSQESVTEQDSKDSTYS | |
| | DKRVEPKSCDKTHTCPPCPAP | | LSSTLTLSKADYEKHKVY | |
| | ELLGGPSVFLFPPKPKDTLMIS | | ACEVTHQGLSSPVTKSF | |
| | RTPEVTCVVVDVSHEDPEVKF | | NRGEC | |
| | NWYVDGVEVHNAKTKPREEQ | | | |
| | YNSTYRVVSVLTVLHQDWLNG | | | |
| | KEYKCKVSNKALPAPIEKTISKA | | | |
| | KGQPREPQVYTLPPSREEMTK | | | |
| | NQVSLTCLVKGFYPSDIAVEW | | | |
| | ESNGQPENNYKTTPPVLDSDG | | | |
| | SFFLYSKLTVDKSRWQQGNVF | | | |
| | SCSVMHEALHNHYTQKSLSLS | | | |
| | PG | | | |

In some embodiments, the anti-IL-11 antibody is a monoclonal antibody. In some embodiments, the anti-IL-11 antibody is a human antibody. In some embodiments, the anti-IL-11 antibody is a chimeric antibody, a bispecific antibody, or a humanized antibody.

In some embodiments, the anti-IL-11 antibody or antigen-binding fragment thereof exhibits thermostability and/or long-term stability. Stability may refer to the length of time over which an antibody or antigen-binding fragment thereof retains its original chemical identity, for example, primary, secondary, and/or tertiary structure including for example upon storage. The stability of an antibody can be determined using standard methods. For example, the stability of an antibody can be determined by measuring the thermal melt (“Tm”) temperature. The Tm is the temperature in degrees Celsius at which ½ of the molecules become unfolded. Typically, the higher the Tm, the more stable the molecule. In exemplary embodiments, the antibody or antigen-binding fragment thereof is refractory to the formation of by-products of the antibody or antigen-binding fragment thereof, for example, high molecular weight aggregation products, low molecular weight degradation or fragmentation products, or mixtures thereof. By-products may include, for example, undesired products, which detract, or diminish the proportion of the antibody or antigen-binding fragment thereof in a given composition. Typical by-products include aggregates of the antibody, fragments of the antibody (for example, produced by degradation of the antibody by deamidation or hydrolysis), or mixtures thereof. High molecular weight polypeptide aggregates may include aggregates of the antibody, fragments of the antibody (for example, produced by degradation of the antibody by, for example, hydrolysis), or mixtures thereof, that then aggregate. For example, for an IgG antibody, such aggregates are greater than about 150 kD. However, in the case of antibody fragments, for example, single-chain antibodies, which typically have a molecular weight of 25 kD, such aggregates would have a molecular weight greater than about 25 kD. Low molecular weight polypeptide degradation products include, for example, fragments of the antibody, for example, brought about by deamidation or hydrolysis. For example, for an IgG antibody, such degradation products are less than about 150 kD. However, in the case of antibody fragments, for example, single-chain antibodies, which typically have a molecular weight of 25 kD, such aggregates would have a molecular weight less than about 25 kD.

In some aspects, assays are provided for determining the stability (e.g., thermostability) of an anti-IL-11 antibody or antigen-binding fragment thereof. For example, the stability of an anti-IL-11 antibody or antigen-binding fragment thereof may be determined using any method known in the art, for example, differential scanning fluorimetry (DSF), circular dichroism (CD), intrinsic protein fluorescence, differential scanning calorimetry, spectroscopy, light scattering (e.g., dynamic light scattering (DLS) and static light scattering (SLS), self-interaction chromatography (SIC). In some embodiments, an anti-IL-11 antibody or antigen-binding fragment thereof of the present disclosure has favorable pharmacokinetic properties. In some aspects, an anti-IL-11 antibody of the present disclosure has favorable manufacturing and biophysical properties, for example yield, quality, stability or solubility.

Variant anti-IL-11 antibodies and antibody fragments thereof can be engineered based on a set of CDRs depicted in Table 1. It is to be understood that in the variant anti-IL-11 antibodies and antigen-binding fragments thereof the amino acid sequence of the CDRs remain unchanged or have minimal changes (e.g., 1-5 changes), but the surrounding regions, e.g., framework regions can be engineered. Amino acid sequence variants of the anti-IL-11 antibody can be prepared by introducing appropriate nucleotide changes into the anti-IL-11 antibody DNA, or by peptide synthesis. Such variants include, for example, deletions from, and/or insertions into and/or substitutions of, residues within the amino acid sequences of the anti-IL-11 antibodies of the examples herein. Any combination of deletions, insertions, and substitutions is made to arrive at the final construct, provided that the final construct possesses the desired characteristics. The amino acid changes also may alter post-translational processes of the human or variant anti-IL-11 antibody, such as changing the number or position of glycosylation sites.

In some embodiments, the present disclosure includes anti-IL-11 antibodies or antigen-binding fragments thereof having a variable heavy chain and a variable light chain, wherein the variable heavy chain amino acid sequence and the variable light chain amino acid sequence are at least 90%, at least 92.5%, at least 95%, at least 98%, or at least 99% identical to the amino acid sequences disclosed in Tables 2-4 provided that the antibody or fragments thereof retain binding to IL-11.

In some embodiments, the present disclosure includes anti-IL-11 antibodies or antigen-binding fragments thereof having a variable heavy chain and a variable light chain, wherein the variable heavy chain amino acid sequence is at least 80%, at least 85%, at least 90%, at least 92.5%, at least 95%, at least 98%, or at least 99% identical to the amino acid sequences of one of SEQ ID NOs: 14, 15, 16, 17, or 18, and wherein the variable light chain amino acid sequence is at least 80%, at least 85%, at least 90%, at least 92.5%, at least 95%, at least 98%, or at least 99% identical to the amino acid sequences of one of SEQ ID NOs: 19, 20, or 21.

In some embodiments, the present disclosure provides anti-IL-11 antibodies having a heavy chain and a light chain, wherein the heavy chain amino acid sequence is at least 95%, at least 98%, or at least 99% identical to one of the amino acid sequences disclosed in Tables 5 and 6, and wherein the light chain amino acid sequence is at least 95%, at least 98%, or at least 99% identical to one of the amino acid sequences disclosed in Tables 5 and 6, provided that the antibody or antigen binding fragment thereof retain binding to IL-11.

In some embodiments, the anti-IL-11 antibodies or antigen-binding fragments thereof comprise a variable heavy chain sequence that comprises an amino acid sequence with at least about 95%, about 96%, about 97%, about 98%, or about 99%, sequence identity to the amino acid sequence set forth in SEQ ID NOs: 14, 15, 16, 17, or 18. In other embodiments, the anti-IL-11 antibodies or antigen-binding fragments thereof retain the binding and/or functional activity of an anti-IL-11 antibody or antibody fragment thereof that comprises the variable heavy chain sequence of SEQ ID NOs: 14, 15, 16, 17, or 18. In still further embodiments, the anti-IL-11 antibodies or antigen-binding fragments thereof comprise the variable heavy chain sequence of SEQ ID NOS: 14, 15, 16, 17, or 18 and have one or more conservative amino acid substitutions, e.g., 1, 2, 3, 4, 5, 1-2, 1-3, 1-4 or 1-5 conservative amino acid substitutions in the heavy chain variable sequence. In yet further embodiments, the one or more conservative amino acid substitutions fall within one or more CDR and/or framework regions in SEQ ID NOs: 14, 15, 16, 17, or 18 (based on the numbering system of Kabat).

In some embodiments, the anti-IL-11 antibody or antigen-binding fragment thereof comprises a variable heavy chain sequence with at least about 95%, about 96%, about 97%, about 98%, or about 99% sequence identity to the anti-IL-11 heavy chain variable region sequence set forth in SEQ ID NOs: 14, 15, 16, 17, or 18 comprises one or more conservative amino acid substitutions in a CDR and/or framework region (based on the numbering system of Kabat), and retains the binding and/or functional activity of an anti-IL-11 antibody or antibody fragment thereof that comprises a variable heavy chain sequence as set forth in SEQ ID NOS: 14, 15, 16, 17, or 18 and a variable light chain sequence as set forth in SEQ ID NOs: 19, 20, or 21.

In some embodiments, the anti-IL-11 antibodies or antigen-binding fragments thereof comprise a variable light chain sequence that comprises an amino acid sequence with at least about 95%, about 96%, about 97%, about 98%, or about 99%, sequence identity to the amino acid sequence set forth in SEQ ID NOs: 19, 20, or 21. In some embodiments, the anti-IL-11 antibodies or antigen-binding fragments thereof retain the binding and/or functional activity of an anti-IL-11 antibody or antigen-binding fragment thereof that comprises the variable light chain sequence of SEQ ID NOs: 19, 20, or 21. In some embodiments, the anti-IL-11 antibodies or antigen-binding fragments thereof comprise the variable light chain sequence of SEQ ID NOS: 19, 20, or 21 and have one or more conservative amino acid substitutions, e.g., 1, 2, 3, 4, 5, 1-2, 1-3, 1-4 or 1-5 conservative amino acid substitutions in the light chain variable sequence. In some embodiments, the one or more conservative amino acid substitutions fall within one or more CDR and/or framework regions in SEQ ID NOs: 19, 20, or 21 (based on the numbering system of Kabat).

In some embodiments, the anti-IL-11 antibody or antigen-binding fragments thereof comprises a variable light chain sequence with at least about 95%, about 96%, about 97%, about 98%, or about 99% sequence identity to the anti-IL-11 light chain variable region sequence set forth in SEQ ID NOs: 19, 20, or 21 comprises one or more conservative amino acid substitutions in a CDR and/or framework region (based on the numbering system of Kabat), and retains the binding and/or functional activity of an anti-IL-11 antibody or antigen-binding fragment thereof that comprises a variable heavy chain sequence as set forth in SEQ ID NOs: 14, 15, 16, 17, or 18 and a variable light chain sequence as set forth in SEQ ID NOs: 19, 20, or 21.

In some embodiments, the anti-IL-11 antibody or antigen-binding fragment thereof specifically binds to IL-11, in particular human, rat, marmoset, rhesus, and cynomolgus monkey (cyno) IL-11, more particularly human IL-11. In some embodiments, the anti-IL-11 antibody or antigen-binding fragment thereof modulates IL-11Rα mediated signaling.

In some embodiments, the anti-IL-11 antibody or antigen-binding fragment thereof has an IC₅₀ of about 0.20 to about 100 nM. In some embodiments, the anti-IL-11 antibody or antigen-binding fragment thereof has an IC₅₀ of about 0.20 nM, about 0.30 nM, about 0.40 nM, about 0.50 nM, about 0.60 nM, about 0.70 nM, about 0.80 nM, about 0.90 nM, about 1.00 nM, about 1.5 nM, about 2.0 nM, about 2.5 nM, about 3.0 nM, about 3.5 nM, about 4.0 nM, about 4.5 nM, about 5.0 nM, about 5.5 nM, about 6.0 nM, about 6.5 nM, about 7.0 nM, about 7.5 nM, about 8.0 nM, about 8.5 nM, about 9.0 nM, about 9.5 nM, about 10.0 nM, about 10.5 nM, about 11.0 nM, about 11.5 nM, about 12.0 nM, about 12.5 nM, about 13.0 nM, about 13.5 nM, about 14.0 nM, about 14.5 nM, or about 15.0 nM.

In some embodiments, the anti-IL-11 antibody or antigen-binding fragment thereof, binds to human IL-11 at a high affinity. In some aspects of the disclosure, the anti-IL-11 antibody or antigen-binding fragment thereof, binds to human IL-11 (e.g., human IL-11 comprising the amino acid sequence set forth in SEQ ID NOs: 48 or 49) at a high affinity, for example at an affinity of 2 nM or less (e.g., about 2 nM, about 1.9 nM, about 1.8 nM, about 1.7 nM, about 1.6 nM, about 1.5 nM, about 1.4 nM, about 1.3 nM, about 1.2 nM, about 1.1 nM, about 1.0 nM, about 0.9 nM, about 0.8 nM, about 0.7 nM, about 0.6 nM, about 0.5 nM, about 0.45 nM, about 0.4 nM, about 0.35 nM, about 0.3 nM, about 0.25 nM, about 0.2 nM, about 0.15 nM, or about 0.10 nM), preferably when measured by surface plasmon resonance.

In some embodiments, the disclosure provides an anti-IL-11 antibody or antigen-binding fragment thereof that blocks the interaction between IL-11 and IL-11Rα. In some embodiments, the anti-IL-11 antibody or antigen-binding fragment thereof blocks IL-11-mediated signaling. In some embodiments, the antibody of the disclosure blocks the binding of IL-11 to IL-11Rα, whereby it decreases IL-11Rα-mediated IL-11 signaling by at least 80%, by at least 85%, by at least 90%, or by at least 95% when compared with a comparator antibody control or in the absence of an anti-IL-11 antibody or antigen-binding fragment of the disclosure. In some embodiments, the comparator antibody control comprises a heavy chain variable region comprising the amino acid sequence of SEQ ID NOs: 14, 15, 16, 17, or 18; and a light chain variable region comprising the amino acid sequence of SEQ ID NOs: 19, 20, or 21.

Whether an anti-IL-11 antibody or antigen-binding fragment thereof specifically binds to a target can be tested with various methods known in the art. These methods include Surface Plasmon Resonance (SPR) or ELISA to detect binding of antibodies to purified proteins, or flow cytometry to detect binding of antibodies to cells. The ability of an antibody to block binding of IL-11 can be measured by detecting purified soluble IL-11 interaction with IL-11Rα-expressing cells. Alternatively, the blocking activity of an antibody can be measured by assessing IL-11Rα phosphorylation and recruitment of gp130. Methods for determining antibody specificity and affinity by competitive inhibition are known in the art. In some embodiments, the anti-IL-11 antibody or antigen-binding fragment thereof recognizes a specific linear and/or conformational “IL-11 antigen epitope” and “IL-11 epitope”.

As used herein, the terms “IL-11 antigen epitope” and “IL-11 epitope” refer to a molecule (e.g., a peptide) or a fragment of a molecule capable of binding to an anti-IL-11 antibody or antigen-binding fragment thereof. These terms further include, for example, an IL-11 antigenic determinant recognized by any of the antibodies or antibody fragments of the present disclosure or key points of contact between the molecule and antibody.

IL-11 antigen epitopes can be included in proteins, protein fragments, peptides or the like. The epitopes are most commonly proteins, short oligopeptides, oligopeptide mimics (e.g., organic compounds that mimic antibody binding properties of the IL-11 antigen), or combinations thereof.

In some aspects of the disclosure, the antibody or antigen-binding fragment thereof recognizes a specific linear or conformational “IL-11 epitope” on a IL-11 protein comprising the amino acid sequence set forth in SEQ ID NOs: 48 or 49. In some aspects of the disclosure, the antibody or antigen-binding fragment thereof recognizes a specific linear or conformational “IL-11 epitope” on an IL-11 protein comprising the amino acid sequence set forth in SEQ ID NO: 48 or 49. In some aspects of the disclosure the antibody or antigen-binding fragment thereof binds to an IL-11 epitope comprising the amino acids: R15, D19, L23, R26, S27 L29, A30, D31, R33, R40, N50, L57, A58, R111, H161, L162, D165, W166, V168, R169, L172, L173, K175, or T176 as set forth in SEQ ID NO: 49.

In some aspects of the disclosure the antibody or antigen-binding fragment thereof binds to the IL-11 epitope comprising the amino acids R15, D19, L23, R26, S27 L29, A30, D31, R33, R40, N50, L57, A58, R111, H161, L162, D165, W166, V168, R169, L172, L173, K175, or T176 as set forth in SEQ ID NO: 49. In some aspects, the antibody or antigen-binding fragment thereof blocks binding of any of Antibody A-O to IL-11.

In some embodiments, the epitope is present on an IL-11 protein set forth in Table 7 below.

TABLE 7
Sequence of IL-11 protein

| Protein | Sequence | SEQ ID NO: |
|---|---|---|
| Human IL-11 (UniProt- | MNCVCRLVLVVLSLWPDTAVAPGPPPGPPRV | 48 |
| P20809-1) | SPDPRAELDSTVLLTRSLLADTRQLAAQLRDK | |
| | FPADGDHNLDSLPTLAMSAGALGALQLPGVL | |
| | TRLRADLLSYLRHVQWLRRAGGSSLKTLEPEL | |
| | GTLQARLDRLLRRLQLLMSRLALPQPPPDPPA | |
| | PPLAPPSSAWGGIRAAHAILGGLHLTLDWAVR | |
| | GLLLLKTRL | |
| Human IL-11 | PGPPPGPPRVSPDPRAELDSTVLLTRSLLADT | 49 |
| | RQLAAQLRDKFPADGDHNLDSLPTLAMSAGA | |
| | LGALQLPGVLTRLRADLLSYLRHVQWLRRAG | |
| | GSSLKTLEPELGTLQARLDRLLRRLQLLMSRL | |
| | ALPQPPPDPPAPPLAPPSSAWGGIRAAHAILG | |
| | GLHLTLDWAVRGLLLLKTRLGGSGHHHHHH | |

The present disclosure also provides an anti-IL-11 antibody or antigen-binding fragment thereof that competes for binding to IL-11 with an anti-IL-11 antibody according to the present disclosure. In some embodiments, the present disclosure provides an anti-IL-11 antibody or antigen-binding fragment thereof that competes for binding to IL-11 with any one of Antibody A-O. Competition assays may be conducted for example as described in PLOS One. 2014; 9(3): e92451 using a biosensor, or PLOS One 2020 Mar. 5; 15(3): e0229206, or by a method disclosed herein.

### Antibody Variants

In certain embodiments, amino acid sequence variants (e.g., antibody variants of the anti-IL-11 antibodies and antigen-binding fragments thereof including one or more amino acid residue alterations) of the antibodies provided herein are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, for example, antigen binding.

In certain embodiments, antibody variants having one or more amino acid substitutions are provided. Sites of interest for substitutional mutagenesis include the HVRs and FRs. Conservative substitutions are shown in Table 8 under the heading of “exemplary amino acid substitutions,” and as further described below in reference to amino acid side chain classes. “Conservative modifications” refer to amino acid modifications that do not significantly affect or alter the binding characteristics of the antibody containing the amino acid sequences. Conservative modifications include amino acid substitutions, additions and deletions. Conservative substitutions are those in which the amino acid is replaced with an amino acid residue having a similar side chain. The families of amino acid residues having similar side chains are well defined and include amino acids with acidic side chains (e.g., aspartic acid, glutamic acid), basic side chains (e.g., lysine, arginine, histidine), nonpolar side chains (e.g., alanine, valine, leucine, isoleucine, proline, phenylalanine, methionine), uncharged polar side chains (e.g., glycine, asparagine, glutamine, cysteine, serine, threonine, tyrosine, tryptophan), aromatic side chains (e.g., phenylalanine, tryptophan, histidine, tyrosine), aliphatic side chains (e.g., glycine, alanine, valine, leucine, isoleucine, serine, threonine), amide (e.g., asparagine, glutamine), beta-branched side chains (e.g., threonine, valine, isoleucine) and sulfur-containing side chains (cysteine, methionine). Furthermore, any native residue in the polypeptide may also be substituted with alanine, as has been previously described for alanine scanning mutagenesis (MacLennan et al. (1998) Acta Physiol Sc and Suppl 643:55-67; Sasaki et al. (1998) Adv Biophys 35:1-24). Amino acid substitutions to the antibodies of the invention may be made by known methods for example by PCR mutagenesis (U.S. Pat. No. 4,683,195). Amino acid substitutions may be introduced into an antibody of interest and the products screened for a desired activity, for example, retained/improved antigen binding, decreased immunogenicity, or improved ADCC or CDC.

TABLE 8
Exemplary amino acid substitutions

| Original | Exemplary |
|---|---|
| Residue | Substitutions |
| Ala (A) | val; leu; ile |
| Arg (R) | lys; gln; asn |
| Asn (N) | gln; his; asp, lys; arg |
| Asp (D) | glu; asn |
| Cys (C) | ser; ala |
| Gln (Q) | asn; glu |
| Glu (E) | asp; gln |
| Gly (G) | ala |
| His (H) | arg; asn; gln; lys; |
| Ile (I) | leu; val; met; ala; phe; norleucine |
| Leu (L) | ile; norleucine; val; met; ala; phe |
| Lys (K) | arg; gln; asn |
| Met (M) | leu; phe; ile |
| Phe (F) | tyr; leu; val; ile; ala |
| Pro (P) | ala |
| Ser (S) | thr |
| Thr (T) | ser |
| Trp (W) | tyr; phe |
| Tyr (Y) | phe; trp; thr; ser |
| Val (V) | leu; ile; met; phe ala; norleucine |

In protein chemistry, it is generally accepted that the biological properties of the antibody can be accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:
(1) hydrophobic: norleucine, met, ala, val, leu, ile;
(2) neutral hydrophilic: cys, ser, thr;
(3) acidic: asp, glu;
(4) basic: asn, gin, his, lys, arg;
(5) residues that influence chain orientation: gly, pro; and
(6) aromatic: trp, tyr, phe.
   Non-conservative substitutions will entail exchanging a member of one of these classes for another class.

In some embodiments, the anti-IL-11 antibody or antigen fragment thereof comprises all six of the CDR regions of Antibody A-O formatted as a chimeric or a humanized antibody.

Antigen-binding fragments, monospecific or multispecific antibodies may be produced by recombinant DNA techniques or by enzymatic or chemical cleavage of intact antibodies. Examples of antibody fragments include but are not limited to Fv, Fab, Fab′, Fab′-SH, F(ab)₂; diabodies; linear antibodies; single-chain antibody molecules (e.g., scFv). “Single-chain Fv” also abbreviated as “sFv” or “scFv” are antibody fragments that comprise the VH and VL antibody domains connected into a single polypeptide chain. One or more of the disclosed anti-IL-11 binding CDRs may be incorporated into a recombinant molecule either covalently or noncovalently to make it an antigen-binding protein in the format of chimeric antibodies, diabodies, triabodies, tetrabodies, and polypeptides that contain at least a portion of an immunoglobulin that is sufficient to confer IL-11 specific binding to the polypeptide.

In certain embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al., TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibody variants with certain improved properties.

In some embodiments, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g., complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, for example, US Patent Publication Nos. U.S. 2003/0157108; U.S. 2004/0093621. Examples of publications related to “defucosylated” or “fucose-deficient” antibody variants include: U.S. 2003/0157108; WO 2000/61739; WO 2001/29246; U.S. 2003/0115614; U.S. 2002/0164328; U.S. 2004/0093621; U.S. 2004/0132140; U.S. 2004/0110704; U.S. 2004/0110282; U.S. 2004/0109865; WO 2003/085119; WO 2003/084570; WO 2005/035586; WO 2005/035778; WO 2005/053742; WO 2002/031140; Okazaki et al., J. Mol. Biol. 336:1239-1249 (2004); Yamane-Ohnuki et al., Biotech. Bioeng. 87:614 (2004). Examples of cell lines capable of producing defucosylated antibodies include Lec13 CHO cells deficient in protein fucosylation (Ripka et al., Arch. Biochem. Biophys. 249:533-545 (1986); U.S. 2003/0157108 A1, Presta, L; and WO 2004/056312 A1, Adams et al., especially at Example 11), and knockout cell lines, such as alpha-1,6-fucosyltransferase gene, FUT8, knockout CHO cells (e.g., Yamane-Ohnuki et al., Biotech. Bioeng. 87:614 (2004); Kanda et al., Biotechnol. Bioeng., 94(4): 680-688 (2006); and WO 2003/085107).

Antibody variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878; U.S. Pat. No. 6,602,684; and U.S. 2005/0123546. Antibody variants with at least one galactose residue in the oligosaccharide attached to the Fc region are also provided. Such antibody variants may have improved CDC function. Such antibody variants are described, for example, in WO 1997/30087; WO 1998/58964; and WO 1999/22764.

In certain embodiments, one or more amino acid modifications may be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid residue alteration (e.g., a substitution) at one or more amino acid positions.

In certain embodiments, the disclosure contemplates an antibody variant that possesses some but not all effector functions, which make it a desirable candidate for applications in which the half-life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity) but retains FcRn binding ability. The primary cells for mediating ADCC, NK cells, express Fc (RIII only, whereas monocytes express Fc (RI, Fc(RII and Fc(RIII. FcR expression on hematopoietic cells is summarized in Table 3 on page 464 of Ravetch and Kinet, Annu. Rev. Immunol. 9:457-492 (1991).

Non-limiting examples of in vitro assays to assess ADCC activity of a molecule of interest are described in U.S. Pat. No. 5,500,362 (see, e.g. Hellstrom et al., Proc. Nat'l Acad. Sci. USA 83:7059-7063 (1986) and Hellstrom, I et al., Proc. Nat'l Acad. Sci. USA 82:1499-1502 (1985); U.S. Pat. No. 5,821,337; and Bruggemann et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, CA; and CYTOTOX 96® non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, for example, in an animal model such as that disclosed in Clynes et al., Proc. Nat'l Acad. Sci. USA 95:652-656 (1998). C1q binding assays may also be carried out to confirm that the antibody is unable to bind C1q and hence lacks CDC activity. See, for example, C1q and C3c binding ELISA in WO 2006/029879 and WO 2005/100402. To assess complement activation, a CDC assay may be performed (see, e.g., Gazzano-Santoro et al., J. Immunol. Methods 202:163 (1996); Cragg et al., Blood 101:1045-1052 (2003); and Cragg et al., Blood 103:2738-2743 (2004)). FcRn binding and in vivo clearance/half-life determinations can also be performed using methods known in the art (see, e.g., Petkova et al., Int'l. Immunol. 18(12): 1759-1769 (2006)).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001)).

In certain embodiments, an antibody variant comprises an Fc region with one or more amino acid substitutions which improve ADCC, e.g., substitutions at positions 298, 333, and/or 334 of the Fc region (EU numbering of residues).

In some embodiments, alterations are made in the Fc region that result in altered (i.e., either improved or diminished) C1q binding and/or Complement Dependent Cytotoxicity (CDC), for example, as described in U.S. Pat. No. 6,194,551, WO 99/51642, and Idusogie et al., J. Immunol. 164:4178-4184 (2000).

Antibodies with increased half-lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in U.S. 2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826). See also Duncan & Winter, Nature 322:738-40 (1988); U.S. Pat. Nos. 5,648,260; 5,624,821; and WO 94/29351 concerning other examples of Fc region variants.

### Polynucleotides, Vectors, and Host Cells

The present disclosure encompasses polynucleotides (e.g., isolated polynucleotides) that comprise a sequence encoding the anti-IL-11 antibody or the antigen-binding fragment thereof as disclosed herein, vectors, and host cells comprising the polynucleotides, and recombinant techniques for production of the antibody or the antigen-binding fragment thereof. The isolated polynucleotides can encode any desired form of the anti-IL-11 antibody including, for example, full length monoclonal antibodies, Fab, Fab′, F(ab′)₂, and Fv fragments, diabodies, linear antibodies, single-chain antibody molecules, and multispecific antibodies formed from antibody fragments.

Some embodiments include isolated polynucleotides that encode the heavy chain variable region of an antibody or antigen-binding fragment thereof having the amino acid sequence of SEQ ID NOs: 14, 15, 16, 17, or 18. Some embodiments include isolated polynucleotides that encode the light chain variable region of an antibody or antigen-binding fragment thereof having the amino acid sequence of any of SEQ ID NOs: 19, 20, or 21.

In some embodiments, the isolated polynucleotide sequence(s) encodes an antibody or antigen-binding fragment thereof comprising:
(i) a variable heavy chain domain comprising SEQ ID NO: 15, and a variable light chain domain comprising SEQ ID NO: 19;
(ii) a variable heavy chain domain comprising SEQ ID NO: 15, and a variable light chain domain comprising SEQ ID NO: 21;
(iii) a variable heavy chain domain comprising SEQ ID NO: 16, and a variable light chain domain comprising SEQ ID NO: 20;
(iv) a variable heavy chain domain comprising SEQ ID NO: 16, and a variable light chain domain comprising SEQ ID NO: 19;
(v) a variable heavy chain domain comprising SEQ ID NO: 16, and a variable light chain domain comprising SEQ ID NO: 21;
(vi) a variable heavy chain domain comprising SEQ ID NO: 17, and a variable light chain domain comprising SEQ ID NO: 20;
(vii) a variable heavy chain domain comprising SEQ ID NO: 17, and a variable light chain domain comprising SEQ ID NO: 19;
(viii) a variable heavy chain domain comprising SEQ ID NO: 17, and a variable light chain domain comprising SEQ ID NO: 21;
(ix) a variable heavy chain domain comprising SEQ ID NO: 18, and a variable light chain domain comprising SEQ ID NO: 20;
(x) a variable heavy chain domain comprising SEQ ID NO: 18, and a variable light chain domain comprising SEQ ID NO: 19;
(xi) a variable heavy chain domain comprising SEQ ID NO: 18, and a variable light chain domain comprising SEQ ID NO: 21;
(xii) a variable heavy chain domain comprising SEQ ID NO: 14, and a variable light chain domain comprising SEQ ID NO: 20;
(xiii) a variable heavy chain domain comprising SEQ ID NO: 14, and a variable light chain domain comprising SEQ ID NO: 19; or
(xiv) a variable heavy chain domain comprising SEQ ID NO: 14, and a variable light chain domain comprising SEQ ID NO: 21.

In some embodiments, the isolated polynucleotide sequence(s) encodes an antibody or antigen-binding fragment thereof comprising:
(i) a variable heavy chain domain that is 90%, 95%, or 99% identical to SEQ ID NO: 15, and a variable light chain domain that is 90%, 95%, or 99% identical to SEQ ID NO: 19;
(ii) a variable heavy chain domain that is 90%, 95%, or 99% identical to SEQ ID NO: 15, and a variable light chain domain that is 90%, 95%, or 99% identical to SEQ ID NO: 21;
(iii) a variable heavy chain domain that is 90%, 95%, or 99% identical to SEQ ID NO: 16, and a variable light domain that is 90%, 95%, or 99% identical to SEQ ID NO: 20;
(iv) a variable heavy chain domain that is 90%, 95%, or 99% identical to SEQ ID NO: 16, and a variable light chain domain that is 90%, 95%, or 99% identical to SEQ ID NO: 19;
(v) a variable heavy chain domain that is 90%, 95%, or 99% identical to SEQ ID NO: 16, and a variable light chain domain that is 90%, 95%, or 99% identical to SEQ ID NO: 21;
(vi) a variable heavy chain domain that is 90%, 95%, or 99% identical to SEQ ID NO: 17, and a variable light chain domain that is 90%, 95%, or 99% identical to SEQ ID NO: 20;
(vii) a variable heavy chain domain that is 90%, 95%, or 99% identical to SEQ ID NO: 17, and a variable light chain domain that is 90%, 95%, or 99% identical to SEQ ID NO: 19;
(viii) a variable heavy chain domain that is 90%, 95%, or 99% identical to SEQ ID NO: 17, and a variable light chain domain that is 90%, 95%, or 99% identical to SEQ ID NO: 21;
(ix) a variable heavy chain domain that is 90%, 95%, or 99% identical to SEQ ID NO: 18, and a variable light chain domain that is 90%, 95%, or 99% identical to SEQ ID NO: 20;
(x) a variable heavy chain domain that is 90%, 95%, or 99% identical to SEQ ID NO: 18, and a variable light chain domain that is 90%, 95%, or 99% identical to SEQ ID NO: 19;
(xi) a variable heavy chain domain that is 90%, 95%, or 99% identical to SEQ ID NO: 18, and a variable light chain domain that is 90%, 95%, or 99% identical to SEQ ID NO: 21;
(xii) a variable heavy chain domain that is 90%, 95%, or 99% identical to SEQ ID NO: 14, and a variable light chain domain that is 90%, 95%, or 99% identical to SEQ ID NO: 20;
(xiii) a variable heavy chain domain that is 90%, 95%, or 99% identical to SEQ ID NO: 14, and a variable light chain domain that is 90%, 95%, or 99% identical to SEQ ID NO: 19; or
(xiv) a variable heavy chain domain that is 90%, 95%, or 99% identical to SEQ ID NO: 14, and a variable light chain domain that is 90%, 95%, or 99% identical to SEQ ID NO: 21.

The polynucleotide(s) that encode an anti-IL-11 antibody or and antigen-binding fragment thereof can be fused to one or more regulatory or control sequences, as known in the art, and can be contained in suitable expression vectors or host cell as known in the art. Each of the polynucleotide molecules encoding the heavy or light chain variable domains can be independently fused to a polynucleotide sequence encoding a constant domain, such as a human constant domain, enabling the production of intact antibodies. Alternatively, polynucleotides, or portions thereof, can be fused together, providing a template for production of a single chain antibody.

For recombinant production, a polynucleotide encoding the antibody is inserted into a replicable vector for cloning (amplification of the DNA) or for expression. Many suitable vectors for expressing the recombinant antibody are available. The vector components generally include, but are not limited to, one or more of the following: a signal sequence, an origin of replication, one or more marker genes, an enhancer element, a promoter, and a transcription termination sequence.

The anti-IL-11 antibodies or antigen-binding fragments thereof can also be produced as fusion polypeptides, in which the antibody or antigen-binding fragment thereof is fused with a heterologous polypeptide, such as a signal sequence or other polypeptide having a specific cleavage site at the amino terminus of the mature protein or polypeptide. The heterologous signal sequence selected is typically one that is recognized and processed (e.g., cleaved by a signal peptidase) by the host cell. For prokaryotic host cells that do not recognize and process the anti-IL-11 antibody signal sequence, the signal sequence can be substituted by a prokaryotic signal sequence. The signal sequence can be, for example, alkaline phosphatase, penicillinase, lipoprotein, heat-stable enterotoxin II leaders, and the like. For yeast secretion, the native signal sequence can be substituted, for example, with a leader sequence obtained from yeast invertase alpha-factor (including Saccharomyces and Kluyveromyces α-factor leaders), acid phosphatase, C. albicans glucoamylase, or the signal described in WO 90/13646. In mammalian cells, mammalian signal sequences as well as viral secretory leaders, for example, the herpes simplex gD signal, can be used. The DNA for such precursor region is ligated in reading frame to DNA encoding the anti-IL-11 antibody or antigen-binding fragment thereof.

Anti-IL-11 antibody or antigen-binding fragment thereof transcription from vectors in mammalian host cells is controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, or from heat-shock promoters, provided such promoters are compatible with the host cell systems.

Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41 P disclosed in DD 266,710 published Apr. 12, 1989), Pseudomonas such as P. aeruginosa, and Streptomyces. One preferred E. coli cloning host is E. coli 294 (ATCC 31,446), although other strains such as E. coli B, E. coli X1776 (ATCC 31,537), and E. coli W3110 (ATCC 27,325) are suitable. These examples are illustrative rather than limiting.

In addition to prokaryotes, eukaryotic microbes such as filamentous fungi or yeast are suitable cloning or expression hosts for anti-IL-11 antibody or antigen-binding fragment thereof encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastors (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of glycosylated an anti-IL-11 antibody or antigen-binding fragment thereof are derived from multicellular organisms. Examples of invertebrate cells include plant and insect cells, including, e.g., numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori (silkworm). A variety of viral strains for transfection are publicly available, e.g., the L-1 variant of Autographa californica NPV and the Bm-5 strain of Bombyx mori NPV, and such viruses may be used, particularly for transfection of Spodoptera frugiperda cells.

Plant cell cultures of cotton, corn, potato, soybean, petunia, tomato, and tobacco can also be utilized as hosts.

The anti-IL-11 antibodies or antigen-binding fragments thereof can also be incorporated in viral vectors, e.g., the polynucleotide encoding for the anti-IL-11 antibody or antigen-binding fragment thereof is introduced into the viral vector and then expressed in the body of the subject after infection with the virus.

In some aspects, expression of the anti-IL-11 antibody or antigen-binding fragment thereof is carried out in vertebrate cells. The propagation of vertebrate cells in culture (tissue culture) has become routine procedure and techniques are widely available. Examples of useful mammalian host cell lines are monkey kidney CV1 line transformed by SV40 (COS-7, ATCC CRL 1651), human embryonic kidney line (293 or 293 cells subcloned for growth in suspension culture, (Graham et al., 1977, J. Gen Virol. 36:59), baby hamster kidney cells (BHK, ATCC CCL 10), Chinese hamster ovary cells/-DHFR1 (CHO, Urlaub et al., 1980, Proc. Natl. Acad. Sci. USA 77:4216; e.g., DG44), mouse sertoli cells (TM4, Mather, 1980, Biol. Reprod. 23:243-251), monkey kidney cells (CV1 ATCC CCL 70), African green monkey kidney cells (VERO-76, ATCC CRL-1587), human cervical carcinoma cells (HELA, ATCC CCL 2), canine kidney cells (MDCK, ATCC CCL 34), buffalo rat liver cells (BRL 3A, ATCC CRL 1442), human lung cells (W138, ATCC CCL 75), human liver cells (Hep G2, HB 8065), mouse mammary tumor (MMT 060562, ATCC CCL51), TR1 cells (Mather et al., 1982, Annals N.Y. Acad. Sci. 383:44-68), MRC 5 cells, FS4 cells, and human hepatoma line (Hep G2).

Also included are nucleic acids that hybridize under low, moderate, and high stringency conditions, in particular under high stringency conditions, as defined herein, to all or a portion (e.g., the portion encoding the variable region) of the nucleotide sequence represented by isolated polynucleotide sequence(s) that encode an anti-IL-11 antibody or antigen-binding fragment thereof. The hybridizing portion of the hybridizing nucleic acid is typically at least 15 (e.g., 20, 25, 30 or 50) nucleotides in length. The hybridizing portion of the hybridizing nucleic acid is at least 80%, e.g., at least 90%, at least 95%, at least 98%, or at least 99% identical to the sequence of a portion or all of a nucleic acid encoding an anti-IL-11 polypeptide (e.g., a heavy chain or light chain variable region), or its complement. Hybridizing nucleic acids of the type described herein can be used, for example, as a cloning probe, a primer, e.g., a PCR primer, or a diagnostic probe. In some aspects, “high stringency conditions” means for probes of at least 100 nucleotides in length, prehybridization and hybridization at 42° C. in 5×SSPE, 0.3% SDS, 200 micrograms/mL sheared and denatured salmon sperm DNA, and 50% formamide, following standard Southern blotting procedures for 12 to 24 hours. The carrier material is finally washed three times each for 15 minutes using 0.2×SSC, 0.2% SDS at 65° C.

### Compositions, Combinations and Administration Thereof

The disclosure provides compositions that comprise an anti-IL-11 antibody or an antigen-binding fragment thereof. Such compositions may be administered to a subject having or at risk of the IL-11 pathway diseases and/or disorders described herein. The disclosure further provides for the use of an anti-IL-11 antibody or an antigen-binding fragment thereof in the manufacture of a medicament for prevention or treatment of an IL-11 pathway disease or disorder. The term “subject” as used herein means any mammalian patient to which an anti-IL-11 antibody or an antigen-binding fragment thereof can be administered, including, e.g., humans and non-human mammals, such as primates, and dogs. Subjects specifically intended for treatment using the methods described herein include humans.

An anti-IL-11 antibody or an antigen-binding fragment thereof may be administered on their own or in combination with one or more additional therapeutic agents, such as state-of-the-art or standard-of-care compounds, such as e.g. cytostatic or cytotoxic substances, cell proliferation inhibitors, anti-angiogenic substances, steroids, immune modulators/checkpoint inhibitors, and the like.

In some aspects, the present disclosure also provides pharmaceutical compositions administered as pharmaceutical compositions comprising a therapeutically effective amount of the anti-IL-11 antibody or an antigen-binding fragment thereof and one or more pharmaceutically compatible ingredients, and optionally one or more additional therapeutic agents.

Some aspects of the disclosure provide a binding molecule of the disclosure for use in the therapy of a pulmonary disease (e.g., an individual suffering from a pulmonary disease or being at risk of developing a pulmonary disease) wherein said therapy comprises one or more pharmacologically active substances.

Some aspects of the disclosure provide the use of one or more active ingredients in the manufacture of a medicament for the therapy of a pulmonary disease (e.g., an individual suffering from a pulmonary disease or being at risk of developing a pulmonary disease) wherein said medicament comprises the binding molecule of the disclosure.

Various delivery systems are known and can be used to administer the anti-IL-11 antibody or an antigen-binding fragment thereof. Methods of introduction include but are not limited to intravitreal, eye drops, intradermal, intramuscular, intraperitoneal, intravenous, subcutaneous, intranasal, epidural, and oral routes. The anti-IL-11 antibody or an antigen-binding fragment thereof can be administered, for example by infusion, bolus or injection, and can be administered together with other biologically active agents. Administration can be systemic or local. Formulations for such injections may be prepared in, for example, prefilled syringes. As such in some aspects of the disclosure, pre-filled syringes are provided that include an anti-IL-11 antibody or an antigen-binding fragment thereof.

To be used in therapy, the anti-IL-11 antibody of the disclosure is formulated into pharmaceutical compositions appropriate to facilitate administration to animals or humans. Typical formulations of the antibody or antigen-binding fragment thereof described herein can be prepared by mixing the antibody or antigen-binding fragment thereof with physiologically acceptable carriers, excipients or stabilizers, in the form of lyophilized or otherwise dried formulations or aqueous solutions or aqueous or non-aqueous suspensions.

In typical embodiments, the pharmaceutical composition is formulated in accordance with routine procedures as a pharmaceutical composition adapted for intravenous or subcutaneous administration to a subject. Typically, compositions for administration by injection are solutions in sterile isotonic aqueous buffer. Where necessary, the pharmaceutical composition can also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachet indicating the quantity of active agent. Where the pharmaceutical is to be administered by infusion, it can be dispensed with an infusion bottle containing sterile pharmaceutical grade water or saline. Where the pharmaceutical is administered by injection, an ampoule of sterile water for injection or saline can be provided so that the ingredients can be mixed prior to administration.

Further, the pharmaceutical composition can be provided as a pharmaceutical kit comprising (a) a container containing an anti-IL-11 antibody or an antigen-binding fragment thereof in lyophilized form and (b) a second container containing a pharmaceutically acceptable diluent (e.g., sterile water) for injection. The pharmaceutically acceptable diluent can be used for reconstitution or dilution of the lyophilized anti-IL-11 antibody or antigen-binding fragment thereof. Optionally associated with such container(s) can be a notice in the form prescribed by a governmental agency regulating the manufacture, use or sale of pharmaceuticals or biological products, which notice reflects approval by the agency of manufacture, use or sale for human administration.

### Therapeutic Uses

The anti-IL-11 antibodies of the disclosure or antigen-binding fragments thereof may be useful for treating and/or preventing an IL-11 pathway disease or disorder. In some embodiments, the anti-IL-11 antibodies of the disclosure or antigen-binding fragments thereof are useful in the preparation of a medicament for the treatment of an IL-11 pathway disease or disorder.

Accordingly, in some embodiments, the disclosure provides a method of modulating the interaction between IL-11 and IL-11Rα in a subject in need thereof comprising administering to said subject a composition comprising an anti-IL-11 antibody or antigen-binding fragment thereof according to the disclosure including, for example, in an amount sufficient to block IL-11Rα-mediated IL-11 signaling in said subject. In some embodiments, the present disclosure provides an anti-IL-11 antibody or antigen-binding fragment thereof according to the present disclosure for use in modulating the interaction between IL-11 and IL-11Rα in a subject. In some embodiments, the disclosure provides the use of an anti-IL-11 antibody or antigen-binding fragment thereof according to the present disclosure in the manufacture of a medicament for modulating the interaction between IL-11 and IL-11Rα in a subject.

In some embodiments, the present disclosure provides a method of treating an IL-11 pathway disease or disorder in a subject in need thereof comprising administering to said subject a composition comprising an anti-IL-11 antibody or antigen-binding fragment thereof according to the present disclosure. In some embodiments, the present disclosure provides an anti-IL-11 antibody or antigen-binding fragment thereof according to the present disclosure for use in treating or preventing an inflammatory disease, autoimmune disease, respiratory/pulmonary disease, infectious disease, or fibrosis in a subject. In some embodiments, the present disclosure provides the use of an anti-IL-11 antibody or antigen-binding fragment thereof according to the present disclosure in the manufacture of a medicament for treating or preventing an inflammatory disease, autoimmune disease, respiratory/pulmonary disease, infectious disease, or fibrosis in a subject.

In some embodiments, the IL-11 pathway disease or disorder is a liver disease or disorder, a lung disease or disorder, a musculoskeletal disease or disorder, a cardiac disease or disorder, a kidney disease or disorder, a metabolic disease or disorder, a cancer, a CNS disease or disorder, a lymphatic disease or disorder, an eye-related disease or disorder, an inflammatory disease or disorder, an autoimmune disease or disorder, or an infectious disease.

In some embodiments, the IL-11 pathway disease or disorder is SLD, MASH, early stage MASH, late stage MASH, other steatotic liver disease conditions (e.g., MASLD, MetALD, ALD, AH, specific etiology SLD, DILI, cryptogenic SLD, monogenic forms of SLD, Chronic liver disease, PBC, PSC, liver cirrhosis, compensated liver cirrhosis, decompensated liver cirrhosis alcohol-induced cirrhosis, steatohepatitis, alcoholic steatohepatitis, steatosis, ARLD, AFL, alcoholic hepatitis, ASH, alcohol-induced liver fibrosis (including early stage alcoholic liver fibrosis and later-stage fibrosis), hepatotoxicity/toxic hepatitis (acute or chronic), chronic active hepatitis, DILI (e.g., intrinsic or idiosyncratic hepatotoxicity, including allergic and nonallergic reaction), APAP-induced hepatotoxicity, ALI, acute liver failure, acute-on-chronic liver failure, acute liver disease, liver damage, hepatitis, viral hepatitis, alcoholic hepatitis, liver IRI, WIR, IDILI, autoimmune liver injury, cholestatic liver disease, HIV-associated liver injury, Wilson's Disease, Haemochromatosis), schistosomal liver disease, radiation-induced liver disease (RILD), iatrogenic causes (e.g., surgical damage to liver tissue), fasting, malnutrition, infection by infectious agents (e.g., hepatitis virus, HIV), cancer or drug interactions, sepsis, herbal or dietary supplements), pulmonary artery hypertension (PAH), COPD (chronic obstructive pulmonary disease), acute respiratory distress syndrome (ARDS), leiomyoma, leiomyosarcoma, progressive massive fibrosis, obliterative bronchiolitis, asbestosis, silicosis, AIDS-associated pulmonary hypertension, sarcoidosis, tumor stroma in lung disease, Hutchinson-Gilford Progeria Syndrome (HGPS), Hermansky-Pudlak Syndrome (HPS), cystic fibrosis, asthma, lung disease, AIDS associated pulmonary hypertension, sarcopenia, muscular dystrophy such as Duchenne muscular dystrophy (DMD), hip replacement failure, Becker's muscular dystrophy (BMD), myopenia, anorexic disorders (protein-energy malnutrition), lipodystrophies (e.g. abnormal or degenerative condition of adipose tissue), cardiomyopathy, cardiac or myocardial fibrosis (e.g., fibrosis in the heart associated with dysfunction of the musculature leading to CHF), hypertrophic cardiomyopathy (HCM), fibrosis of the atrium, fibrosis of the ventricle, myocardial fibrosis, endomyocardial fibrosis, myocardial infarction, fibrotic vascular disease, hypertensive heart disease, tubulointerstitial and glomerular fibrosis, atherosclerosis, cerebral infarcts, hypertension, vascular aneurysm, aortic aneurysm, familial thoracic aortic aneurysm syndrome, cerebral aneurysm, vascular stenosis and restenosis, renal artery stenosis, atrial fibrillation, Marfan's syndrome, Furlong's syndrome, Sphrintzen-Goldberg syndrome, Loeys-Dietz syndrome, arterial tortuosity syndrome, plexiform lesions, fibromuscular dysplasia (FMD), supravalvular stenosis, telangiectasia, varicose veins, Brugada syndrome, arrhythmogenic right ventricular cardiomyopathy (ARVC), dilated cardiomyopathy (DCM), ventricular fibrillation, myocarditis, kidney disease/renal disease (e.g., chronic kidney disease, renal fibrosis, nephritic syndrome, Alport's syndrome, HIV associated nephropathy, polycystic kidney disease, Fabry's disease, diabetic nephropathy, chronic glomerulonephritis, lupus nephritis/nephritis associated with systemic lupus, or kidney interstitial fibrosis (IF)), focal and segmental glomerulosclerosis (FSGS), IgA nephropathy, crescentic glomerulonephritis, kidney injury, acute kidney injury/renal failure, acute kidney failure, acute kidney disease, chronic kidney disease, kidney damage, autoimmune kidney injury, membranous nephropathy, nephrotoxicity, bladder disease, obesity, type 2 diabetes (T2D), type 1 diabetes (T1D)/type 1 diabetes mellitus, pre-diabetes, being overweight, metabolic syndrome, gestational diabetes, insulin deficiency, pancreas injury, pregnancy-associated hyperglycemia, cholestasis (i.e., a reduced flow of bile from the liver to the duodenum), cholestatic liver disease (e.g., primary biliary cholangitis (PBC)), hyperglycemia, hyperlipidemia, hypertriglyceridemia, hypercholesterolemia, wasting, “mild muscle wasting disease,” cachexia, pre-cachexia, refractory cachexia, sarcopenia, muscular dystrophies, steatosis (macrovesicular or microvesicular), lipotoxicity (including cells of the liver, kidney, heart and/or skeletal muscle), fatty liver disease, metabolic dysfunction-associated steatotic liver disease (MASLD), metabolic dysfunction-associated steatotic liver (MASL), insulin deficiency, insulin resistance, lipodystrophy, lipohypertrophy, lipoatrophy, chemotherapy-associated weight loss, pancreatic insufficiency, acute pancreatitis, chronic pancreatitis, hyperglucagonemia, Glioblastoma, breast cancer, colon cancer, gastric cancer, Leukemia, NSCLC, prostate cancer, hepatocellular carcinoma, epithelial cell cancer, gastrointestinal cancer (including oesophageal cancer, stomach cancer, pancreatic cancer, liver cancer (HCC), gallbladder cancer, colorectal cancer, anal cancer, gastrointestinal carcinoid tumour), lung cancer (including non-small cell lung cancer (NSCLC) and small cell lung cancer (SCLC)), Crohn's disease, ulcerative colitis, achalasia, dysphagia, diarrhoea, constipation, inflammatory bowel disease (IBD), bowel stricture, pyloric stenosis, celiac disease, irritable bowel syndrome (IBS, irritable bowel syndrome), diverticulitis, microscopic colitis, primary sclerosing cholangitis (PSC), systemic sclerosis/scleroderma, progressive systemic sclerosis (PSS), chronic graft versus host disease, nephrogenic systemic fibrosis, cutis keloid, arthrofibrosis, Dupuytren's contracture, mediastinal fibrosis, retroperitoneal fibrosis, Peyronie's disease, adhesive capsulitis, fibrotic pre-neoplastic and fibrotic neoplastic disease, fibrosis induced by chemical or environmental insult (e.g., cancer chemotherapy, pesticides, radiation/cancer radiotherapy), arthritis, multiple sclerosis, gliosis, Alzheimer's disease, myelofibrosis, myeloproliferative diseases, aplastic anemia, thyroid eye disease, retinopathy, diabetic retinopathy, chronic pulmonary hypertension, Grave's ophthalmopathy, epiretinal fibrosis (e.g. diabetic retinopathy (DR)), glaucoma, subretinal fibrosis (e.g. associated with macular degeneration (e.g. wet or dry age-related macular degeneration (AMD))), macular edema, drusen formation, choroidal neovascularization (CNV), post-surgical fibrosis (e.g. of the posterior capsule following cataract surgery, or of the bleb following trabeculectomy for glaucoma), conjunctival fibrosis, subconjunctival fibrosis, retinal fibrosis (e.g. associated with wet age-related macular degeneration (AMD)), an Interstitial lung disease (ILD), including idiopathic pulmonary fibrosis (IPF), progressive pulmonary fibrosis (PPF), fibrotic ILA, scleroderma affecting the lung, or scleroderma-associated ILD, an inflammatory disease or disorder, an autoimmune disease or disorder, or an infectious disease.

In some embodiments, the IL-11 pathway disease or disorder is a liver disease or disorder including, for example, MASH, early stage MASH, late stage MASH, other steatotic liver disease conditions (e.g., MASLD, MetALD, ALD, AH, specific etiology SLD, DILI, cryptogenic SLD, monogenic forms of SLD, Chronic liver disease, PBC, PSC, liver cirrhosis, compensated liver cirrhosis, decompensated liver cirrhosis alcohol-induced cirrhosis, steatohepatitis, alcoholic steatohepatitis, steatosis, ARLD, AFL, alcoholic hepatitis, ASH, alcohol-induced liver fibrosis (including early stage alcoholic liver fibrosis and later-stage fibrosis), hepatotoxicity/toxic hepatitis (acute or chronic), chronic active hepatitis, DILI (e.g., intrinsic or idiosyncratic hepatotoxicity, including allergic and nonallergic reaction), APAP-induced hepatotoxicity, ALI, acute liver failure, acute-on-chronic liver failure, acute liver disease, liver damage, hepatitis, viral hepatitis, alcoholic hepatitis, liver IRI, WIR, IDILI, autoimmune liver injury, cholestatic liver disease, HIV-associated liver injury, Wilson's Disease, Haemochromatosis, schistosomal liver disease, radiation-induced liver disease (RILD), iatrogenic causes (e.g., surgical damage to liver tissue), fasting, malnutrition, infection by infectious agents (e.g., hepatitis virus, HIV), cancer or drug interactions, sepsis, herbal or dietary supplements), or pulmonary artery hypertension (PAH). In some embodiments, treating comprises stopping disease progression. In some embodiments, treating comprises promoting cirrhosis regression.

In some embodiments, the IL-11 pathway disease or disorder is a musculoskeletal disease or disorder including, for example, sarcopenia, muscular dystrophy such as Duchenne muscular dystrophy (DMD), hip replacement failure, Becker's muscular dystrophy (BMD), myopenia, anorexic disorders (protein-energy malnutrition), or lipodystrophies (e.g. abnormal or degenerative condition of adipose tissue).

In some embodiments, the IL-11 pathway disease or disorder is a cardiac disease or disorder including, for example, cardiomyopathy, cardiac or myocardial fibrosis (e.g., fibrosis in the heart associated with dysfunction of the musculature leading to CHF), hypertrophic cardiomyopathy (HCM), fibrosis of the atrium, fibrosis of the ventricle, myocardial fibrosis, endomyocardial fibrosis, myocardial infarction, fibrotic vascular disease, hypertensive heart disease, tubulointerstitial and glomerular fibrosis, atherosclerosis, cerebral infarcts, hypertension, vascular aneurysm, aortic aneurysm, familial thoracic aortic aneurysm syndrome, cerebral aneurysm, vascular stenosis and restenosis, renal artery stenosis, atrial fibrillation, Marfan's syndrome, Furlong's syndrome, Sphrintzen-Goldberg syndrome, Loeys-Dietz syndrome, arterial tortuosity syndrome, plexiform lesions, fibromuscular dysplasia (FMD), supravalvular stenosis, telangiectasia, varicose veins, Brugada syndrome, arrhythmogenic right ventricular cardiomyopathy (ARVC), dilated cardiomyopathy (DCM), ventricular fibrillation, myocarditis, or fibrosis of the ventricle.

In some embodiments, the IL-11 pathway disease or disorder is a kidney disease or disorder including, for example, chronic kidney disease, renal fibrosis, nephritic syndrome, Alport's syndrome, HIV associated nephropathy, polycystic kidney disease, Fabry's disease, diabetic nephropathy, chronic glomerulonephritis, lupus nephritis/nephritis associated with systemic lupus, or kidney interstitial fibrosis (IF), focal and segmental glomerulosclerosis (FSGS), IgA nephropathy, crescentic glomerulonephritis, kidney injury, acute kidney injury/renal failure, acute kidney failure, acute kidney disease, chronic kidney disease, kidney damage, autoimmune kidney injury, membranous nephropathy, or nephrotoxicity.

In some embodiments, the IL-11 pathway disease or disorder is a metabolic disease or disorder including, for example, obesity, type 2 diabetes (T2D), type 1 diabetes (T1D)/type 1 diabetes mellitus, pre-diabetes, being overweight, metabolic syndrome, gestational diabetes, insulin deficiency, pancreas injury, pregnancy-associated hyperglycemia, cholestasis (i.e., a reduced flow of bile from the liver to the duodenum), cholestatic liver disease (e.g., primary biliary cholangitis (PBC)), hyperglycemia, hyperlipidaemia, hypertriglyceridemia, hypercholesterolemia, wasting, “mild muscle wasting disease”, cachexia, pre-cachexia, refractory cachexia, sarcopenia, muscular dystrophies, steatosis (macrovesicular or microvesicular), lipotoxicity (including cells of the liver, kidney, heart and/or skeletal muscle), fatty liver disease, metabolic dysfunction-associated steatotic liver disease (MASLD), metabolic dysfunction-associated steatotic liver (MASL), insulin deficiency, insulin resistance, lipodystrophy, lipohypertrophy, lipoatrophy, chemotherapy-associated weight loss, pancreatic insufficiency, acute pancreatitis, chronic pancreatitis, or hyperglucagonemia.

In some embodiments, the IL-11 pathway disease or disorder is a cancer including, for example, glioblastoma, breast cancer, colon cancer, gastric cancer, leukemia, NSCLC, prostate cancer, hepatocellular carcinoma, epithelial cell cancer, gastrointestinal cancer (including oesophageal cancer, stomach cancer, pancreatic cancer, liver cancer (HCC), gallbladder cancer, colorectal cancer, anal cancer, gastrointestinal carcinoid tumour), or lung cancer (including non-small cell lung cancer (NSCLC) and small cell lung cancer (SCLC)).

In some embodiments, the IL-11 pathway disease or disorder is a lung disease including, for example, interstitial lung disease (ILD), including idiopathic pulmonary fibrosis (IPF), progressive pulmonary fibrosis (PPF), fibrotic ILA, scleroderma affecting the lung, scleroderma-associated ILD, chronic pulmonary hypertension, pulmonary artery hypertension (PAH), COPD (chronic obstructive pulmonary disease), acute respiratory distress syndrome (ARDS), leiomyoma, leiomyosarcoma, progressive massive fibrosis, obliterative bronchiolitis, asbestosis, silicosis, AIDS-associated pulmonary hypertension, sarcoidosis, tumor stroma in lung disease, Hutchinson-Gilford Progeria Syndrome (HGPS), Hermansky-Pudlak Syndrome (HPS), cystic fibrosis, asthma, lung disease, or AIDS associated pulmonary hypertension. In some embodiments, treating comprises preventing development of progression in individuals with ILD or fibrotic ILA at risk of becoming fibrotic ILD. In some embodiments, treating comprises restoring lost lung function. In some embodiments, treating comprises stabilizing lung function by halting the pathologic cycle.

In some embodiments, the IL-11 pathway disease or disorder is a gastrointestinal disease or disorder including, for example, Crohn's disease, ulcerative colitis, achalasia, dysphagia, diarrhoea, constipation, inflammatory bowel disease (IBD), bowel stricture, pyloric stenosis, coeliac disease, irritable bowel syndrome (IBS, irritable bowel syndrome), diverticulitis, microscopic colitis, or primary sclerosing cholangitis (PSC).

In some embodiments, the IL-11 pathway disease or disorder is a multi-system disease or disorder including, for example, systemic sclerosis/scleroderma, progressive systemic sclerosis (PSS), chronic graft versus host disease, nephrogenic systemic fibrosis, cutis keloid, arthrofibrosis, Dupuytren's contracture, mediastinal fibrosis, retroperitoneal fibrosis, myelofibrosis, Peyronie's disease, adhesive capsulitis, fibrotic pre-neoplastic and fibrotic neoplastic disease, fibrosis induced by chemical or environmental insult (e.g., cancer chemotherapy, pesticides, radiation/cancer radiotherapy), or arthritis.

In some embodiments, the IL-11 pathway disease or disorder is a CNS disease or disorder including, for example, multiple sclerosis, gliosis, or Alzheimer's disease.

In some embodiments, the IL-11 pathway disease or disorder is a lymphatic disease or disorder including, for example, myelofibrosis, myeloproliferative diseases, or aplastic anemia.

In some embodiments, the IL-11 pathway disease or disorder is an eye disease or disorder including, for example, thyroid eye disease, retinopathy, diabetic retinopathy, Grave's ophthalmopathy, epiretinal fibrosis (e.g. diabetic retinopathy (DR)), glaucoma, subretinal fibrosis (e.g. associated with macular degeneration (e.g. wet or dry age-related macular degeneration (AMD))), macular edema, drusen formation, choroidal neovascularization (CNV), post-surgical fibrosis (e.g. of the posterior capsule following cataract surgery, or of the bleb following trabeculectomy for glaucoma), conjunctival fibrosis, subconjunctival fibrosis, or retinal fibrosis (e.g. associated with wet age-related macular degeneration (AMD)).

In some embodiments, the IL-11 pathway disease or disorder is an inflammatory or autoimmune disease including, for example, allergic asthma, bronchial inflammation, atopic dermatitis, allergic rhinitis, ocular allergic diseases, multiple sclerosis, systemic lupus erythematosus, rheumatoid arthritis, chronic active hepatitis, type 1 diabetes mellitus, celiac disease, Grave's disease, uveitis, pemphigus, psoriasis, Crohn's disease, ulcerative colitis, inflammatory bowel disease, anaemia, autoimmune thyroiditis.

In some embodiments, the IL-11 pathway disease or disorder is an infectious disease including, for example, bacterial, viral, fungal, or parasitic infection, Helicobacter pylori infection, Mycobacterium tuberculosis infection, EBV infection, HPV infection.

In some embodiments, the IL-11 pathway disease or disorder is MASH, early stage MASH, late stage MASH, sarcopenia, cardiomyopathy, cardiac or myocardial fibrosis, kidney disease/renal disease (e.g., chronic kidney disease, renal fibrosis, nephritic syndrome, Alport's syndrome, HIV associated nephropathy, polycystic kidney disease, Fabry's disease, diabetic nephropathy, chronic glomerulonephritis, lupus nephritis/nephritis associated with systemic lupus, or kidney IF), sarcopenia, or ILD, including IPF, PPF, fibrotic ILA, scleroderma affecting the lung, or scleroderma-associated ILD.

In some embodiments, the IL-11 pathway disease or disorder is a liver disease selected from the group consisting of: MASH, early-stage MASH, and late-stage MASH.

In some embodiments, the IL-11 pathway disease or disorder is a musculoskeletal or metabolic disease such as sarcopenia.

In some embodiments, the IL-11 pathway disease or disorder is a cardiac disease selected from the groups consisting of: cardiomyopathy, cardiac fibrosis, and myocardial fibrosis.

In some embodiments, the IL-11 pathway disease or disorder is a kidney disease/renal disease (e.g., chronic kidney disease, renal fibrosis, nephritic syndrome, Alport's syndrome, HIV associated nephropathy, polycystic kidney disease, Fabry's disease, diabetic nephropathy, nephrotoxicity, chronic glomerulonephritis, lupus nephritis/nephritis associated with systemic lupus, or kidney IF).

In some embodiments, the IL-11 pathway disease or disorder is a lung disease selected from the group consisting of: interstitial lung disease (ILD), including idiopathic pulmonary fibrosis (IPF), progressive pulmonary fibrosis (PPF), fibrotic ILA, scleroderma affecting the lung, and scleroderma-associated ILD.

In some embodiments, the IL-11 pathway disease or disorder is a lung disease selected from the group consisting of: idiopathic pulmonary fibrosis (IPF) and progressive pulmonary fibrosis (PPF).

### Non-Therapeutic Uses

The antibodies and antigen-binding fragments disclosed herein are useful for non-therapeutic purposes including, for example, as affinity purification agents. In this process, the antibodies or antigen-binding fragments thereof are immobilized on a solid phase such a Protein A resin, using methods well known in the art. The immobilized antibody is contacted with a sample containing the IL-11 protein (or fragment thereof) to be purified, and thereafter the support is washed with a suitable solvent that will remove substantially all the material in the sample except the IL-11 protein, which is bound to the immobilized antibody. Finally, the support is washed with another suitable solvent that will release the IL-11 protein from the antibody.

The IL-11 antibodies and antigen-binding fragments thereof of the disclosure are also useful in diagnostic assays to detect and/or quantify IL-11 protein, for example, detecting IL-11 expression in specific cells, tissues, or serum.

It will be advantageous in some embodiments, for example, for diagnostic purposes to label the antibody or antigen-binding fragment thereof with a detectable moiety. Numerous detectable labels are available, including radioisotopes, fluorescent labels, enzyme substrate labels, quantum dots and the like. The label may be indirectly conjugated with the antibody using various known techniques. For example, the antibody can be conjugated with biotin and any of the three broad categories of labels mentioned above can be conjugated with avidin, or vice versa. Biotin binds selectively to avidin and thus, the label can be conjugated with the antibody in this indirect manner. Alternatively, to achieve indirect conjugation of the label with the antibody, the antibody can be conjugated with a small hapten (such as digoxin) and one of the different types of labels mentioned above is conjugated with an anti-hapten antibody (e.g., anti-digoxin antibody). Thus, indirect conjugation of the label with the antibody can be achieved.

### Diagnostic Kits

An anti-IL-11 antibody or antigen-binding fragment thereof can be used in a diagnostic kit, e.g., a packaged combination of reagents in predetermined amounts with instructions for performing the diagnostic assay, such as an assay for the detection of IL-11, preferably full-length IL-11. Accordingly, also provided herein is the use of the antibody or antigen-binding fragment thereof as described herein for diagnosis of an IL-11 pathway disorder. Where the antibody is labeled with an enzyme, the kit may include substrates and cofactors required by the enzyme such as a substrate precursor that provides the detectable chromophore or fluorophore. In addition, other additives may be included such as stabilizers, buffers (for example a block buffer or lysis buffer), and the like. The relative amounts of the various reagents may be varied widely to provide for concentrations in solution of the reagents that substantially optimize the sensitivity of the assay. The reagents may be provided as dry powders, usually lyophilized, including excipients that on dissolution will provide a reagent solution having the appropriate concentration.

### Articles of Manufacture

In some aspects of the disclosure, an article of manufacture containing materials useful for the treatment, prevention and/or diagnosis of the disease or disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is an antibody of the disclosure. The label or package insert indicates that the composition is used for treating the condition of choice. The article of manufacture in this embodiment of the disclosure may further comprise a package insert indicating that the compositions can be used to treat a particular condition. Alternatively, or additionally, the article of manufacture may further comprise a second (or third) container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### Illustration of Subject Technology as Clauses

Various examples of aspects are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identifications.

Clause 1: An anti-interleukin-11 (IL-11) antibody or an antigen-binding fragment thereof comprising:
a heavy chain variable region (VH) comprising the amino acid sequence of SEQ ID NO: 1 (H-CDR1) or SEQ ID NO: 2 (H-CDR1), the amino acid sequence of SEQ ID NO: 3 (H-CDR2), SEQ ID NO: 4 (H-CDR2), SEQ ID NO: 5 (H-CDR2), or SEQ ID NO: 6 (H-CDR2), and the amino acid sequence of SEQ ID NO: 7 (H-CDR3); and
a light chain variable region (VL) comprising the amino acid sequence of SEQ ID NO: 9 (L-CDR1), the amino acid sequence of SEQ ID NO: 10 (L-CDR2), SEQ ID NO: 11 (L-CDR2), or SEQ ID NO: 12 (L-CDR2) and the amino acid sequence of SEQ ID NO: 13 (L-CDR3).

Clause 2: An anti-interleukin-11 (IL-11) antibody or an antigen-binding fragment thereof comprising:
a heavy chain variable region (VH) comprising the amino acid sequence of SEQ ID NO: 1 (H-CDR1), the amino acid sequence of SEQ ID NO: 3 (H-CDR2), and the amino acid sequence of SEQ ID NO: 7 (H-CDR3); and
a light chain variable region (VL) comprising the amino acid sequence of SEQ ID NO: 9 (L-CDR1), the amino acid sequence of SEQ ID NO: 10 (L-CDR2), and the amino acid sequence of SEQ ID NO: 13 (L-CDR3).

Clause 3: The anti-IL-11 antibody or antigen-binding fragment thereof according to clause 1 or clause 2, wherein the antibody or antigen-binding fragment thereof comprises a VH comprising the amino acid sequence of SEQ ID NOs: 14, 15, 16, 17, or 18.

Clause 4: The anti-IL-11 antibody or antigen-binding fragment thereof according to clause 1 or clause 2, wherein the antibody or antigen-binding fragment thereof comprises a VH that has about 80%, about 85%, about 90%, about 95%, about 96%, about 97%, about 98%, about 99% or about 100% sequence identity to the amino acid sequence of SEQ ID NO: 14, 15, 16, 17, or 18.

Clause 5: The anti-IL-11 antibody or antigen-binding fragment thereof according to clause 4, wherein the antibody or antigen-binding fragment thereof comprises a VH having the amino acid sequence of SEQ ID NO: 14, 15, 16, 17, or 18.

Clause 6: The anti-IL-11 antibody or antigen-binding fragment thereof according to clause 1 or clause 2, wherein the antibody or antigen-binding fragment thereof comprises a VL comprising the amino acid sequence of SEQ ID NOs: 19, 20, or 21.

Clause 7: The anti-IL-11 antibody or antigen-binding fragment thereof according to clause 1 or clause 2, wherein the antibody or antigen-binding fragment thereof comprises a VL that has about 80%, about 85%, about 90%, about 95%, about 96%, about 97%, about 98%, about 99% or about 100% sequence identity to the amino acid sequence of SEQ ID NO: 19, 20, or 21.

Clause 8: The anti-IL-11 antibody or antigen-binding fragment thereof according to clause 7, wherein the antibody or antigen-binding fragment thereof comprises a VL having the amino acid sequence of SEQ ID NO: 19, 20, or 21.

Clause 9: The anti-IL-11 antibody or antigen-binding fragment thereof according to clause 1 or clause 2, wherein the antibody or antigen-binding fragment thereof comprises a VH comprising the amino acid sequence of SEQ ID NOs: 14, 15, 16, 17, or 18; and a VL comprising the amino acid sequence of SEQ ID NOs: 19, 20, or 21.

Clause 10: The anti-IL-11 antibody or antigen-binding fragment thereof according to clause 9, wherein the antibody or antigen-binding fragment thereof comprises:
a) a VH comprising the amino acid sequence of SEQ ID NO: 14 and a VL comprising the amino acid sequence of SEQ ID NO: 19;
b) a VH comprising the amino acid sequence of SEQ ID NO: 15 and a VL comprising the amino acid sequence of SEQ ID NO: 19;
c) a VH comprising the amino acid sequence of SEQ ID NO: 15 and a VL comprising the amino acid sequence of SEQ ID NO: 21;
d) a VH comprising the amino acid sequence of SEQ ID NO: 16 and a VL comprising the amino acid sequence of SEQ ID NO: 20;
e) a VH comprising the amino acid sequence of SEQ ID NO: 16 and a VL comprising the amino acid sequence of SEQ ID NO: 19;
f) a VH comprising the amino acid sequence of SEQ ID NO: 16 and a VL comprising the amino acid sequence of SEQ ID NO: 21;
g) a VH comprising the amino acid sequence of SEQ ID NO: 17 and a VL comprising the amino acid sequence of SEQ ID NO: 20;
h) a VH comprising the amino acid sequence of SEQ ID NO: 17 and a VL comprising the amino acid sequence of SEQ ID NO: 19;
i) a VH comprising the amino acid sequence of SEQ ID NO: 17 and a VL comprising the amino acid sequence of SEQ ID NO: 21;
j) a VH comprising the amino acid sequence of SEQ ID NO: 18 and a VL comprising the amino acid sequence of SEQ ID NO: 20;
k) a VH comprising the amino acid sequence of SEQ ID NO: 18 and a VL comprising the amino acid sequence of SEQ ID NO: 19;
l) a VH comprising the amino acid sequence of SEQ ID NO: 18 and a VL comprising the amino acid sequence of SEQ ID NO: 21;
m) a VH comprising the amino acid sequence of SEQ ID NO: 14 and a VL comprising the amino acid sequence of SEQ ID NO: 20; or
n) a VH comprising the amino acid sequence of SEQ ID NO: 14 and a VL comprising the amino acid sequence of SEQ ID NO: 21.

Clause 11: The anti-IL-11 antibody or antigen-binding fragment thereof according to clause 1 or clause 2, wherein the antibody or antigen-binding fragment thereof comprises a VH that has about 80%, about 85%, about 90%, about 95%, about 96%, about 97%, about 98%, about 99% or about 100% sequence identity to the amino acid sequence of SEQ ID NO: 14; and a VL that has about 80%, about 85%, about 90%, about 95%, about 96%, about 97%, about 98%, about 99% or about 100% sequence identity to the amino acid sequence of SEQ ID NO: 19.

Clause 12: The anti-IL-11 antibody or antigen-binding fragment thereof according to any of the preceding clauses, wherein the antibody or antigen-binding fragment thereof comprises a heavy chain constant region (CH) that has about 80%, about 85%, about 90%, about 95%, about 96%, about 97%, about 98%, about 99% or about 100% sequence identity to the amino acid sequence of SEQ ID NO: 22.

Clause 13: The anti-IL-11 antibody or antigen-binding fragment thereof according to any of the preceding clauses, wherein the antibody or antigen-binding fragment thereof comprises a CH having the amino acid sequence of SEQ ID NO: 22.

Clause 14: The anti-IL-11 antibody or antigen-binding fragment thereof according to any of the preceding clauses, wherein the antibody or antigen-binding fragment thereof comprises a light chain constant region (CL) that has about 80%, about 85%, about 90%, about 95%, about 96%, about 97%, about 98%, about 99% or about 100% sequence identity to the amino acid sequence of SEQ ID NO: 24.

Clause 15: The anti-IL-11 antibody or antigen-binding fragment thereof according to any of the preceding clauses, wherein the antibody or antigen-binding fragment thereof comprises a CL having the amino acid sequence of SEQ ID NO: 24.

Clause 16: The anti-IL-11 antibody or antigen-binding fragment thereof according to any of the preceding clauses, wherein the antibody or antigen-binding fragment thereof comprises: a CH that has about 80%, about 85%, about 90%, about 95%, about 96%, about 97%, about 98%, about 99% or about 100% sequence identity to the amino acid sequence of SEQ ID NO: 22; and a CL that has about 80%, about 85%, about 90%, about 95%, about 96%, about 97%, about 98%, about 99% or about 100% sequence identity to the amino acid sequence of SEQ ID NO: 24.

Clause 17: The anti-IL-11 antibody or antigen-binding fragment thereof according to any of the preceding clauses, wherein the antibody or antigen-binding fragment thereof comprises a CH having the amino acid sequence of SEQ ID NO: 22; and a CL having the amino acid sequence of SEQ ID NO: 24.

Clause 18: The anti-IL-11 antibody or antigen-binding fragment thereof according to any one of clauses 12, 13, 16, or 17, wherein the antibody or antigen-binding fragment thereof comprises from N- to C-terminus the VH domain and the CH region, wherein the VH domain is operably linked to the CH domain.

Clause 19: The anti-IL-11 antibody or antigen-binding fragment thereof according to any one of clauses 14, 15, 16, or 17, wherein the antibody or antigen-binding fragment thereof comprises from N- to C-terminus the VL domain and the CL region, wherein the VL domain is operably linked to the CL region.

Clause 20: The anti-IL-11 antibody or antigen-binding fragment thereof according to any one of clauses 16 or 17, wherein the antibody or antigen-binding fragment thereof comprises: from N- to C-terminus the VH domain and the CH region, wherein the VH domain is operably linked to the CH region; and wherein the antibody or antigen-binding fragment thereof comprises: from N- to C-terminus the VL domain and the CL region, wherein the VL domain is operably linked to the CL region.

Clause 21: The anti-IL-11 antibody or antigen-binding fragment thereof according to any of the preceding clauses comprising a heavy chain that comprises the amino acid sequence of SEQ ID NO: 26, 27, 28, 29, or 30.

Clause 22: The anti-IL-11 antibody or antigen-binding fragment thereof according to any of the preceding clauses comprising a light chain that comprises the amino acid sequence of SEQ ID NO: 31, 32, or 33.

Clause 23: The anti-IL-11 antibody or antigen-binding fragment thereof according to any of the preceding clauses comprising:
(i) a heavy chain that comprises the amino acid sequence of SEQ ID NO: 26, and a light chain that comprises the amino acid sequence of SEQ ID NO: 31;
(ii) a heavy chain that comprises the amino acid sequence of SEQ ID NO: 27, and a light chain that comprises the amino acid sequence of SEQ ID NO: 31;
(iii) a heavy chain that comprises the amino acid sequence of SEQ ID NO: 27, and a light chain that comprises the amino acid sequence of SEQ ID NO: 33;
(iv) a heavy chain that comprises the amino acid sequence of SEQ ID NO: 28, and a light chain that comprises the amino acid sequence of SEQ ID NO: 32;
(v) a heavy chain that comprises the amino acid sequence of SEQ ID NO: 28, and a light chain that comprises the amino acid sequence of SEQ ID NO: 31;
(vi) a heavy chain that comprises the amino acid sequence of SEQ ID NO: 28, and a light chain that comprises the amino acid sequence of SEQ ID NO: 33;
(vii) a heavy chain that comprises the amino acid sequence of SEQ ID NO: 29, and a light chain that comprises the amino acid sequence of SEQ ID NO: 32;
(viii) a heavy chain that comprises the amino acid sequence of SEQ ID NO: 29, and a light chain that comprises the amino acid sequence of SEQ ID NO: 31;
(xi) a heavy chain that comprises the amino acid sequence of SEQ ID NO: 29, and a light chain that comprises the amino acid sequence of SEQ ID NO: 33;
(x) a heavy chain that comprises the amino acid sequence of SEQ ID NO: 30, and a light chain that comprises the amino acid sequence of SEQ ID NO: 32;
(xi) a heavy chain that comprises the amino acid sequence of SEQ ID NO: 30, and a light chain that comprises the amino acid sequence of SEQ ID NO: 31;
(xii) a heavy chain that comprises the amino acid sequence of SEQ ID NO: 30, and a light chain that comprises the amino acid sequence of SEQ ID NO: 33;
(xiii) a heavy chain that comprises the amino acid sequence of SEQ ID NO: 26, and a light chain that comprises the amino acid sequence of SEQ ID NO: 32; or
(xiv) a heavy chain that comprises the amino acid sequence of SEQ ID NO: 26, and a light chain that comprises the amino acid sequence of SEQ ID NO: 33.

Clause 24: The anti-IL-11 antibody or antigen-binding fragment thereof according to any of the preceding clauses, wherein the antibody is a monoclonal antibody.

Clause 25: The anti-IL-11 antibody or antigen-binding fragment thereof according to any of the preceding clauses, wherein the antibody is humanized.

Clause 26: The anti-IL-11 antibody or antigen-binding fragment thereof according to any of the preceding clauses, wherein the antibody or antigen-binding fragment thereof binds to human IL-11 at a KD≤0.50 nM.

Clause 27: The anti-IL-11 antibody according to any of the preceding clauses for use as a medicament.

Clause 28: A polynucleotide encoding the VH and/or the VL according to any one of clauses 1-11.

Clause 29: A polynucleotide encoding the heavy chain and/or the light chain according to any one of clauses 21-23.

Clause 30: A set of polynucleotides comprising a first polynucleotide and a second polynucleotide, wherein the first polynucleotide encodes the VH according to any one of clauses 1-5; and wherein the second polynucleotide encodes the VL according to any one of clauses 1, 2, or 6-8.

Clause 31: A set of polynucleotides comprising a first polynucleotide and a second polynucleotide, wherein the first polynucleotide encodes the heavy chain according to clause 21; and wherein the second polynucleotide encodes the light chain according to clause 22.

Clause 32: A vector comprising the polynucleotide according to any one of clauses 28 or 29 or the set of polynucleotides according to any one of clauses 30 or 31.

Clause 33: A host cell comprising the polynucleotide according to any one of clauses 28 or 29, the set of polynucleotides according to any one of clauses 30 or 31, or the vector according to clause 32.

Clause 34: The host cell of clause 33, wherein the cell is a mammalian cell.

Clause 35: A method for the production of the anti-IL-11 antibody or antigen-binding fragment thereof according to any one of clauses 1 to 26, comprising the steps:
(a) cultivating the host cell according to clause 33 or clause 34 under conditions suitable for expression of the anti-IL-11 antibody or antigen-binding fragment; and
(b) recovering the anti-IL-11 antibody or antigen-binding fragment.

Clause 36: The method of clause 35, further comprising the step of purifying the anti-IL-11 antibody or antigen-binding fragment thereof.

Clause 37: The method of clause 36, further comprising the step of formulating the anti-IL-11 antibody or antigen-binding fragment into a pharmaceutical composition.

Clause 38: A diagnostic kit or diagnostic method comprising the anti-human IL-11 antibody or antigen-binding fragment according to any one of clauses 1 to 26.

Clause 39: A pharmaceutical composition comprising the anti-IL-11 antibody or antigen-binding fragment thereof according to any one of clauses 1 to 26 and a pharmaceutically acceptable carrier.

Clause 40: The pharmaceutical composition of clause 39 further comprising one or more additional therapeutic agents.

Clause 41: A method of treating idiopathic pulmonary fibrosis (IPF) or progressive pulmonary fibrosis (PPF) in a subject in need thereof, the method comprising: administering to the subject a therapeutically effective amount of the anti-IL-11 antibody or antigen-binding fragment thereof according to any one of clauses 1 to 26 or the pharmaceutical composition of clauses 39 or 40.

Clause 42: An anti-IL-11 antibody or antigen-binding fragment thereof according to any one of clauses 1 to 26, or a pharmaceutical composition of clauses 39 or 40, for use in treating idiopathic pulmonary fibrosis (IPF) or progressive pulmonary fibrosis (PPF).

Clause 43: Use of the anti-IL-11 antibody or antigen-binding fragment according to any one of clauses 1 to 26, or a pharmaceutical composition of clauses 39 or 40, in manufacture of a medicament for treating idiopathic pulmonary fibrosis (IPF) or progressive pulmonary fibrosis (PPF).

Clause 44: The method according to clause 41, the anti-IL-11 antibody or antigen-binding fragment according to clause 42, or the use of the anti-IL-11 antibody or antigen-binding fragment according to clause 43, wherein said antibody or antigen-binding fragment thereof is administered intravenously.

Clause 45: An anti-human IL-11 antibody or antigen-binding fragment, wherein when bound to human IL-11, the antibody or antigen-binding fragment binds to amino acid residues R15, D19, L23, R26, S27 L29, A30, D31, R33, R40, N50, L57, A58, R111, H161, L162, D165, W166, V168, R169, L172, L173, K175, or T176 as set forth in SEQ ID NO: 49, and wherein the antibody blocks binding of IL-11 to IL-11Rα.

Clause 46: A method of treating and/or preventing an IL-11 pathway disease or disorder in a subject in need thereof, the method comprising: administering to the subject a therapeutically effective amount of the anti-IL-11 antibody or antigen-binding fragment thereof according to any one of clauses 1 to 26 or the pharmaceutical composition of clauses 39 or 40.

Clause 47: An anti-IL-11 antibody or antigen-binding fragment thereof according to any one of clauses 1 to 26, or a pharmaceutical composition of clauses 39 or 40, for use in treating and/or preventing an IL-11 pathway disease or disorder.

Clause 48: Use of the anti-IL-11 antibody or antigen-binding fragment according to any one of clauses 1 to 26, or a pharmaceutical composition of clauses 39 or 40, in manufacture of a medicament for treating and/or preventing an IL-11 pathway disease or disorder.

Clause 49: The method according to clause 46, the anti-IL-11 antibody or antigen-binding fragment thereof according to clause 47, or the use according to clause 48, wherein the IL-11 pathway disease or disorder is an inflammatory disease, autoimmune disease, respiratory disease, infectious disease, pulmonary disease, or fibrosis.

Clause 50: The method according to clause 46, the anti-IL-11 antibody or antigen-binding fragment thereof according to clause 47, or the use according to clause 48, wherein the IL-11 pathway disease or disorder is a liver disease or disorder, a lung disease or disorder, a musculoskeletal disease or disorder, a cardiac disease or disorder, a kidney disease or disorder, a metabolic disease or disorder, a cancer, a CNS disease or disorder, a lymphatic disease or disorder, an eye-related disease or disorder, an inflammatory disease or disorder, an autoimmune disease or disorder, or an infectious disease.

Clause 51: The method according to clause 46, the anti-IL-11 antibody or antigen-binding fragment thereof according to clause 47, or the use according to clause 48, wherein the IL-11 pathway disease or disorder is SLD, MASH, early stage MASH, late stage MASH, other steatotic liver disease conditions (e.g., MASLD, MetALD, ALD, AH, specific etiology SLD, DILI, cryptogenic SLD, monogenic forms of SLD, Chronic liver disease, PBC, PSC, liver cirrhosis, compensated liver cirrhosis, decompensated liver cirrhosis alcohol-induced cirrhosis, steatohepatitis, alcoholic steatohepatitis, steatosis, ARLD, AFL, alcoholic hepatitis, ASH, alcohol-induced liver fibrosis (including early stage alcoholic liver fibrosis and later-stage fibrosis), hepatotoxicity/toxic hepatitis (acute or chronic), chronic active hepatitis, DILI (e.g., intrinsic or idiosyncratic hepatotoxicity, including allergic and nonallergic reaction), APAP-induced hepatotoxicity, ALI, acute liver failure, acute-on-chronic liver failure, acute liver disease, liver damage, hepatitis, viral hepatitis, alcoholic hepatitis, liver IRI, WIR, IDILI, autoimmune liver injury, cholestatic liver disease, HIV-associated liver injury, Wilson's Disease, Haemochromatosis), schistosomal liver disease, radiation-induced liver disease (RILD), iatrogenic causes (e.g., surgical damage to liver tissue), fasting, malnutrition, infection by infectious agents (e.g., hepatitis virus, HIV), cancer or drug interactions, sepsis, herbal or dietary supplements), pulmonary artery hypertension (PAH), COPD (chronic obstructive pulmonary disease), acute respiratory distress syndrome (ARDS), leiomyoma, leiomyosarcoma, progressive massive fibrosis, obliterative bronchiolitis, asbestosis, silicosis, AIDS-associated pulmonary hypertension, sarcoidosis, tumor stroma in lung disease, Hutchinson-Gilford Progeria Syndrome (HGPS), Hermansky-Pudlak Syndrome (HPS), cystic fibrosis, asthma, lung disease, AIDS associated pulmonary hypertension, sarcopenia, muscular dystrophy such as Duchenne muscular dystrophy (DMD), hip replacement failure, Becker's muscular dystrophy (BMD), myopenia, anorexic disorders (protein-energy malnutrition), lipodystrophies (e.g. abnormal or degenerative condition of adipose tissue), cardiomyopathy, cardiac or myocardial fibrosis (e.g., fibrosis in the heart associated with dysfunction of the musculature leading to CHF), hypertrophic cardiomyopathy (HCM), fibrosis of the atrium, fibrosis of the ventricle, myocardial fibrosis, endomyocardial fibrosis, myocardial infarction, fibrotic vascular disease, hypertensive heart disease, tubulointerstitial and glomerular fibrosis, atherosclerosis, cerebral infarcts, hypertension, vascular aneurysm, aortic aneurysm, familial thoracic aortic aneurysm syndrome, cerebral aneurysm, vascular stenosis and restenosis, renal artery stenosis, atrial fibrillation, Marfan's syndrome, Furlong's syndrome, Sphrintzen-Goldberg syndrome, Loeys-Dietz syndrome, arterial tortuosity syndrome, plexiform lesions, fibromuscular dysplasia (FMD), supravalvular stenosis, telangiectasia, varicose veins, Brugada syndrome, arrhythmogenic right ventricular cardiomyopathy (ARVC), dilated cardiomyopathy (DCM), ventricular fibrillation, myocarditis, kidney disease/renal disease (e.g., chronic kidney disease, renal fibrosis, nephritic syndrome, Alport's syndrome, HIV associated nephropathy, polycystic kidney disease, Fabry's disease, diabetic nephropathy, chronic glomerulonephritis, lupus nephritis/nephritis associated with systemic lupus, or kidney interstitial fibrosis (IF)), focal and segmental glomerulosclerosis (FSGS), IgA nephropathy, crescentic glomerulonephritis, kidney injury, acute kidney injury/renal failure, acute kidney failure, acute kidney disease, chronic kidney disease, kidney damage, autoimmune kidney injury, membranous nephropathy, nephrotoxicity, bladder disease, obesity, type 2 diabetes (T2D), type 1 diabetes (T1D)/type 1 diabetes mellitus, pre-diabetes, being overweight, metabolic syndrome, gestational diabetes, insulin deficiency, pancreas injury, pregnancy-associated hyperglycemia, cholestasis (i.e., a reduced flow of bile from the liver to the duodenum), cholestatic liver disease (e.g., primary biliary cholangitis (PBC)), hyperglycemia, hyperlipidaemia, hypertriglyceridemia, hypercholesterolemia, wasting, “mild muscle wasting disease,” cachexia, pre-cachexia, refractory cachexia, sarcopenia, muscular dystrophies, steatosis (macrovesicular or microvesicular), lipotoxicity (including cells of the liver, kidney, heart and/or skeletal muscle), fatty liver disease, metabolic dysfunction-associated steatotic liver disease (MASLD), metabolic dysfunction-associated steatotic liver (MASL), insulin deficiency, insulin resistance, lipodystrophy, lipohypertrophy, lipoatrophy, chemotherapy-associated weight loss, pancreatic insufficiency, acute pancreatitis, chronic pancreatitis, hyperglucagonemia, Glioblastoma, breast cancer, colon cancer, gastric cancer, Leukemia, NSCLC, prostate cancer, hepatocellular carcinoma, epithelial cell cancer, gastrointestinal cancer (including oesophageal cancer, stomach cancer, pancreatic cancer, liver cancer (HCC), gallbladder cancer, colorectal cancer, anal cancer, gastrointestinal carcinoid tumour), lung cancer (including non-small cell lung cancer (NSCLC) and small cell lung cancer (SCLC)), Crohn's disease, ulcerative colitis, achalasia, dysphagia, diarrhoea, constipation, inflammatory bowel disease (IBD), bowel stricture, pyloric stenosis, coeliac disease, irritable bowel syndrome (IBS, irritable bowel syndrome), diverticulitis, microscopic colitis, primary sclerosing cholangitis (PSC), systemic sclerosis/scleroderma, progressive systemic sclerosis (PSS), chronic graft versus host disease, nephrogenic systemic fibrosis, cutis keloid, arthrofibrosis, Dupuytren's contracture, mediastinal fibrosis, retroperitoneal fibrosis, Peyronie's disease, adhesive capsulitis, fibrotic pre-neoplastic and fibrotic neoplastic disease, fibrosis induced by chemical or environmental insult (e.g., cancer chemotherapy, pesticides, radiation/cancer radiotherapy), arthritis, multiple sclerosis, gliosis, Alzheimer's disease, myelofibrosis, myeloproliferative diseases, aplastic anemia, thyroid eye disease, retinopathy, diabetic retinopathy, chronic pulmonary hypertension, Grave's ophthalmopathy, epiretinal fibrosis (e.g. diabetic retinopathy (DR)), glaucoma, subretinal fibrosis (e.g. associated with macular degeneration (e.g. wet or dry age-related macular degeneration (AMD))), macular edema, drusen formation, choroidal neovascularization (CNV), post-surgical fibrosis (e.g. of the posterior capsule following cataract surgery, or of the bleb following trabeculectomy for glaucoma), conjunctival fibrosis, subconjunctival fibrosis, retinal fibrosis (e.g. associated with wet age-related macular degeneration (AMD)), an Interstitial lung disease (ILD), including idiopathic pulmonary fibrosis (IPF), progressive pulmonary fibrosis (PPF), fibrotic ILA, scleroderma affecting the lung, or scleroderma-associated ILD, an inflammatory disease or disorder, an autoimmune disease or disorder, or an infectious disease.

Clause 52: The method according to clause 46, the anti-IL-11 antibody or antigen-binding fragment thereof according to clause 47, or the use according to clause 48, wherein the IL-11 pathway disease or disorder is a liver disease or disorder including, for example, MASH, early stage MASH, late stage MASH, other steatotic liver disease conditions (e.g., MASLD, MetALD, ALD, AH, specific etiology SLD, DILI, cryptogenic SLD, monogenic forms of SLD, Chronic liver disease, PBC, PSC, liver cirrhosis, compensated liver cirrhosis, decompensated liver cirrhosis alcohol-induced cirrhosis, steatohepatitis, alcoholic steatohepatitis, steatosis, ARLD, AFL, alcoholic hepatitis, ASH, alcohol-induced liver fibrosis (including early stage alcoholic liver fibrosis and later-stage fibrosis), hepatotoxicity/toxic hepatitis (acute or chronic), chronic active hepatitis, DILI (e.g., intrinsic or idiosyncratic hepatotoxicity, including allergic and nonallergic reaction), APAP-induced hepatotoxicity, ALI, acute liver failure, acute-on-chronic liver failure, acute liver disease, liver damage, hepatitis, viral hepatitis, alcoholic hepatitis, liver IRI, WIR, IDILI, autoimmune liver injury, cholestatic liver disease, HIV-associated liver injury, Wilson's Disease, Haemochromatosis, schistosomal liver disease, radiation-induced liver disease (RILD), iatrogenic causes (e.g., surgical damage to liver tissue), fasting, malnutrition, infection by infectious agents (e.g., hepatitis virus, HIV), cancer or drug interactions, sepsis, herbal or dietary supplements), or pulmonary artery hypertension (PAH).

Clause 53: The method according to clause 46, the anti-IL-11 antibody or antigen-binding fragment thereof according to clause 47, or the use according to clause 48, wherein the IL-11 pathway disease or disorder is a musculoskeletal disease or disorder including, for example, sarcopenia, muscular dystrophy such as Duchenne muscular dystrophy (DMD), hip replacement failure, Becker's muscular dystrophy (BMD), myopenia, anorexic disorders (protein-energy malnutrition), or lipodystrophies (e.g. abnormal or degenerative condition of adipose tissue).

Clause 54: The method according to clause 46, the anti-IL-11 antibody or antigen-binding fragment thereof according to clause 47, or the use according to clause 48, wherein the IL-11 pathway disease or disorder is a cardiac disease or disorder including, for example, cardiomyopathy, cardiac or myocardial fibrosis (e.g., fibrosis in the heart associated with dysfunction of the musculature leading to CHF), hypertrophic cardiomyopathy (HCM), fibrosis of the atrium, fibrosis of the ventricle, myocardial fibrosis, endomyocardial fibrosis, myocardial infarction, fibrotic vascular disease, hypertensive heart disease, tubulointerstitial and glomerular fibrosis, atherosclerosis, cerebral infarcts, hypertension, vascular aneurysm, aortic aneurysm, familial thoracic aortic aneurysm syndrome, cerebral aneurysm, vascular stenosis and restenosis, renal artery stenosis, atrial fibrillation, Marfan's syndrome, Furlong's syndrome, Sphrintzen-Goldberg syndrome, Loeys-Dietz syndrome, arterial tortuosity syndrome, plexiform lesions, fibromuscular dysplasia (FMD), supravalvular stenosis, telangiectasia, varicose veins, Brugada syndrome, arrhythmogenic right ventricular cardiomyopathy (ARVC), dilated cardiomyopathy (DCM), ventricular fibrillation, myocarditis, or fibrosis of the ventricle.

Clause 55: The method according to clause 46, the anti-IL-11 antibody or antigen-binding fragment thereof according to clause 47, or the use according to clause 48, wherein the IL-11 pathway disease or disorder is a kidney disease or disorder including, for example, chronic kidney disease, renal fibrosis, nephritic syndrome, Alport's syndrome, HIV associated nephropathy, polycystic kidney disease, Fabry's disease, diabetic nephropathy, chronic glomerulonephritis, lupus nephritis/nephritis associated with systemic lupus, or kidney interstitial fibrosis (IF), focal and segmental glomerulosclerosis (FSGS), IgA nephropathy, crescentic glomerulonephritis, kidney injury, acute kidney injury/renal failure, acute kidney failure, acute kidney disease, chronic kidney disease, kidney damage, autoimmune kidney injury, membranous nephropathy, or nephrotoxicity.

Clause 56: The method according to clause 46, the anti-IL-11 antibody or antigen-binding fragment thereof according to clause 47, or the use according to clause 48, wherein the IL-11 pathway disease or disorder is a metabolic disease or disorder including, for example, obesity, type 2 diabetes (T2D), type 1 diabetes (T1D)/type 1 diabetes mellitus, pre-diabetes, being overweight, metabolic syndrome, gestational diabetes, insulin deficiency, pancreas injury, pregnancy-associated hyperglycemia, cholestasis (i.e., a reduced flow of bile from the liver to the duodenum), cholestatic liver disease (e.g., primary biliary cholangitis (PBC)), hyperglycemia, hyperlipidaemia, hypertriglyceridemia, hypercholesterolemia, wasting, “mild muscle wasting disease”, cachexia, pre-cachexia, refractory cachexia, sarcopenia, muscular dystrophies, steatosis (macrovesicular or microvesicular), lipotoxicity (including cells of the liver, kidney, heart and/or skeletal muscle), fatty liver disease, metabolic dysfunction-associated steatotic liver disease (MASLD), metabolic dysfunction-associated steatotic liver (MASL), metabolic dysfunction-associated steatohepatitis (MASH), insulin deficiency, insulin resistance, lipodystrophy, lipohypertrophy, lipoatrophy, chemotherapy-associated weight loss, pancreatic insufficiency, acute pancreatitis, chronic pancreatitis, or hyperglucagonemia.

Clause 57: The method according to clause 46, the anti-IL-11 antibody or antigen-binding fragment thereof according to clause 47, or the use according to clause 48, wherein the IL-11 pathway disease or disorder is a cancer including, for example, glioblastoma, breast cancer, colon cancer, gastric cancer, leukemia, NSCLC, prostate cancer, hepatocellular carcinoma, epithelial cell cancer, gastrointestinal cancer (including oesophageal cancer, stomach cancer, pancreatic cancer, liver cancer (HCC), gallbladder cancer, colorectal cancer, anal cancer, gastrointestinal carcinoid tumour), or lung cancer (including non-small cell lung cancer (NSCLC) and small cell lung cancer (SCLC)).

Clause 58: The method according to clause 46, the anti-IL-11 antibody or antigen-binding fragment thereof according to clause 47, or the use according to clause 48, wherein the IL-11 pathway disease or disorder is a lung disease including, for example, interstitial lung disease (ILD), including idiopathic pulmonary fibrosis (IPF), progressive pulmonary fibrosis (PPF), fibrotic ILA, scleroderma affecting the lung, scleroderma-associated ILD, chronic pulmonary hypertension, pulmonary artery hypertension (PAH), COPD (chronic obstructive pulmonary disease), acute respiratory distress syndrome (ARDS), leiomyoma, leiomyosarcoma, progressive massive fibrosis, obliterative bronchiolitis, asbestosis, silicosis, AIDS-associated pulmonary hypertension, sarcoidosis, tumor stroma in lung disease, Hutchinson-Gilford Progeria Syndrome (HGPS), Hermansky-Pudlak Syndrome (HPS), cystic fibrosis, asthma, lung disease, or AIDS associated pulmonary hypertension.

Clause 59: The method according to clause 46, the anti-IL-11 antibody or antigen-binding fragment thereof according to clause 47, or the use according to clause 48, wherein the IL-11 pathway disease or disorder is a gastrointestinal disease or disorder including, for example, Crohn's disease, ulcerative colitis, achalasia, dysphagia, diarrhoea, constipation, inflammatory bowel disease (IBD), bowel stricture, pyloric stenosis, coeliac disease, irritable bowel syndrome (IBS, irritable bowel syndrome), diverticulitis, microscopic colitis, or primary sclerosing cholangitis (PSC).

Clause 60: The method according to clause 46, the anti-IL-11 antibody or antigen-binding fragment thereof according to clause 47, or the use according to clause 48, wherein the IL-11 pathway disease or disorder is a multi-system disease or disorder including, for example, systemic sclerosis/scleroderma, progressive systemic sclerosis (PSS), chronic graft versus host disease, nephrogenic systemic fibrosis, cutis keloid, arthrofibrosis, Dupuytren's contracture, mediastinal fibrosis, retroperitoneal fibrosis, myelofibrosis, Peyronie's disease, adhesive capsulitis, fibrotic pre-neoplastic and fibrotic neoplastic disease, fibrosis induced by chemical or environmental insult (e.g., cancer chemotherapy, pesticides, radiation/cancer radiotherapy), or arthritis.

Clause 61: The method according to clause 46, the anti-IL-11 antibody or antigen-binding fragment thereof according to clause 47, or the use according to clause 48, wherein the IL-11 pathway disease or disorder is a CNS disease or disorder including, for example, multiple sclerosis, gliosis, or Alzheimer's disease.

Clause 62: The method according to clause 46, the anti-IL-11 antibody or antigen-binding fragment thereof according to clause 47, or the use according to clause 48, wherein the IL-11 pathway disease or disorder is a lymphatic disease or disorder including, for example, myelofibrosis, myeloproliferative diseases, or aplastic anemia.

Clause 63: The method according to clause 46, the anti-IL-11 antibody or antigen-binding fragment thereof according to clause 47, or the use according to clause 48, wherein the IL-11 pathway disease or disorder is an eye disease or disorder including, for example, thyroid eye disease, retinopathy, diabetic retinopathy, Grave's ophthalmopathy, epiretinal fibrosis (e.g. diabetic retinopathy (DR)), glaucoma, subretinal fibrosis (e.g. associated with macular degeneration (e.g. wet or dry age-related macular degeneration (AMD))), macular edema, drusen formation, choroidal neovascularization (CNV), post-surgical fibrosis (e.g. of the posterior capsule following cataract surgery, or of the bleb following trabeculectomy for glaucoma), conjunctival fibrosis, subconjunctival fibrosis, or retinal fibrosis (e.g. associated with wet age-related macular degeneration (AMD)).

Clause 64: The method according to clause 46, the anti-IL-11 antibody or antigen-binding fragment thereof according to clause 47, or the use according to clause 48, wherein the IL-11 pathway disease or disorder is an inflammatory or autoimmune disease including, for example, allergic asthma, bronchial inflammation, atopic dermatitis, allergic rhinitis, ocular allergic diseases, multiple sclerosis, systemic lupus erythematosus, rheumatoid arthritis, chronic active hepatitis, type 1 diabetes mellitus, celiac disease, Grave's disease, uveitis, pemphigus, psoriasis, Crohn's disease, ulcerative colitis, inflammatory bowel disease, anaemia, autoimmune thyroiditis.

Clause 65: The method according to clause 46, the anti-IL-11 antibody or antigen-binding fragment thereof according to clause 47, or the use according to clause 48, wherein the IL-11 pathway disease or disorder is an infectious disease including, for example, bacterial, viral, fungal, or parasitic infection, Helicobacter pylori infection, Mycobacterium tuberculosis infection, EBV infection, HPV infection.

Clause 66: The method according to clause 46, the anti-IL-11 antibody or antigen-binding fragment thereof according to clause 47, or the use according to clause 48, wherein the IL-11 pathway disease or disorder is MASH, early stage MASH, late stage MASH, sarcopenia, cardiomyopathy, cardiac or myocardial fibrosis, kidney disease/renal disease (e.g., chronic kidney disease, renal fibrosis, nephritic syndrome, Alport's syndrome, HIV associated nephropathy, polycystic kidney disease, Fabry's disease, diabetic nephropathy, chronic glomerulonephritis, lupus nephritis/nephritis associated with systemic lupus, or kidney IF), sarcopenia, or ILD, including IPF, PPF, fibrotic ILA, scleroderma affecting the lung, or scleroderma-associated ILD.

Clause 67: The method according to clause 46, the anti-IL-11 antibody or antigen-binding fragment thereof according to clause 47, or the use according to clause 48, wherein the IL-11 pathway disease or disorder is a liver disease selected from the group consisting of: MASH, early-stage MASH, and late-stage MASH.

Clause 68: The method according to clause 46, the anti-IL-11 antibody or antigen-binding fragment thereof according to clause 47, or the use according to clause 48, wherein the IL-11 pathway disease or disorder is a musculoskeletal or metabolic disease such as sarcopenia.

Clause 69: The method according to clause 46, the anti-IL-11 antibody or antigen-binding fragment thereof according to clause 47, or the use according to clause 48, wherein the IL-11 pathway disease or disorder is a cardiac disease selected from the groups consisting of: cardiomyopathy, cardiac fibrosis, and myocardial fibrosis.

Clause 70: The method according to clause 46, the anti-IL-11 antibody or antigen-binding fragment thereof according to clause 47, or the use according to clause 48, wherein the IL-11 pathway disease or disorder is a kidney disease/renal disease (e.g., chronic kidney disease, renal fibrosis, nephritic syndrome, Alport's syndrome, HIV associated nephropathy, polycystic kidney disease, Fabry's disease, diabetic nephropathy, nephrotoxicity, chronic glomerulonephritis, lupus nephritis/nephritis associated with systemic lupus, or kidney IF).

Clause 71: The method according to clause 46, the anti-IL-11 antibody or antigen-binding fragment thereof according to clause 47, or the use according to clause 48, wherein the IL-11 pathway disease or disorder is a lung disease selected from the group consisting of: interstitial lung disease (ILD), including idiopathic pulmonary fibrosis (IPF), progressive pulmonary fibrosis (PPF), fibrotic ILA, scleroderma affecting the lung, and scleroderma-associated ILD.

Clause 72: The method according to clause 46, the anti-IL-11 antibody or antigen-binding fragment thereof according to clause 47, or the use according to clause 48, wherein the IL-11 pathway disease or disorder is metabolic dysfunction-associated steatohepatitis (MASH).

### EXAMPLES

The present disclosure is now described with reference to the following Examples. These Examples are provided for the purpose of illustration only and the disclosure should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the anti-IL-11 antibodies and antigen-binding fragments thereof of the present disclosure and practice the claimed methods. The following working examples specifically point out the embodiments of the present disclosure and are not to be construed as limiting in any way the remainder of the disclosure.

### Example 1. Generation of Anti-IL-11 Antibodies

Antibodies to human IL-11 were generated by removing potential liabilities from the light and heavy chain of a parental antibody (Antibody A). In particular, for D59/S60, a light chain liability site of L-CDR2, D59 was changed to glutamic acid (E) and alanine (A) with glutamic acid being the minimal change amino acid substitution and alanine being the germline residue. Likewise, in the heavy chain, N55/G56, a potential deamidation site, was changed to glutamine (Q), glycine (G), or serine(S). Additionally, in the heavy chain, D31/Y32, a weak iso-asparate isomerization site, was changed to serine(S). Notably, in silico algorithms (e.g., Molecular Operating Environment) failed to identify VK-CDR2 D59/S60 or VH-CDR1 D31Y32 as isomerization liabilities. Thus, a total of three light chain and five heavy chain variable regions were generated. The amino acid sequence of the light chain and heavy chain variable regions are provided in Table 9 below.

TABLE 9
Light and Heavy Chain Variable Regions

| Variable | | |
|---|---|---|
| Regions | Amino Acid Sequence | SEQ ID NO: |
| IgK | DIVLTQSPASLALSPGERATLSCRASKSVSTSGYSYIHWY | 20 |
| wild-type | QQKPGQAPRLLIYLASNLDSGVPARFSGSGSGTDFTLTIS | |
| (parental) | SLEEEDFATYYCQHSRDLPPTFGQGTKLEIK | |
| IgK D59E | DIVLTQSPASLALSPGERATLSCRASKSVSTSGYSYIHWY | 19 |
| variant | QQKPGQAPRLLIYLASNLESGVPARFSGSGSGTDFTLTIS | |
| | SLEEEDFATYYCQHSRDLPPTFGQGTKLEIK | |
| IgK D59A | DIVLTQSPASLALSPGERATLSCRASKSVSTSGYSYIHWY | 21 |
| variant | QQKPGQAPRLLIYLASNLASGVPARFSGSGSGTDFTLTIS | |
| | SLEEEDFATYYCQHSRDLPPTFGQGTKLEIK | |
| IgG1 VH | EVQLVQSGAEVKKPGASVKISCKASGYTFTDYNMDWVKQ | 15 |
| wild-type | APGQRLEWIGDINPHNGGPIYNQKFTGRATLTVDKSASTA | |
| (parental) | YMELSSLRSEDTAVYYCARGELGHWYFDVWGQGTTVTV | |
| | SS | |
| IgG1 VH | EVQLVQSGAEVKKPGASVKISCKASGYTFTSYNMDWVKQ | 16 |
| D31S | APGQRLEWIGDINPHNGGPIYNQKFTGRATLTVDKSASTA | |
| variant | YMELSSLRSEDTAVYYCARGELGHWYFDVWGQGTTVTV | |
| | SS | |
| IgG1 VH | EVQLVQSGAEVKKPGASVKISCKASGYTFTDYNMDWVKQ | 18 |
| N55Q | APGQRLEWIGDINPHQGGPIYNQKFTGRATLTVDKSASTA | |
| variant | YMELSSLRSEDTAVYYCARGELGHWYFDVWGQGTTVTV | |
| | SS | |
| IgG1 VH | EVQLVQSGAEVKKPGASVKISCKASGYTFTDYNMDWVKQ | 17 |
| N55G | APGQRLEWIGDINPHGGGPIYNQKFTGRATLTVDKSASTA | |
| variant | YMELSSLRSEDTAVYYCARGELGHWYFDVWGQGTTVTV | |
| | SS | |
| IgG1 VH | EVQLVQSGAEVKKPGASVKISCKASGYTFTDYNMDWVKQ | 14 |
| N55S | APGQRLEWIGDINPHSGGPIYNQKFTGRATLTVDKSASTA | |
| variant | YMELSSLRSEDTAVYYCARGELGHWYFDVWGQGTTVTV | |
| | SS | |

The three light chain variable regions and the five heavy chain variable regions in Table 9 above were engineered into a human VK constant region having the amino acid sequence set forth in SEQ ID NO: 24 and a human IgG1 constant region having the amino acid sequence set forth in SEQ ID NO: 22, respectively. Pairs of light chains and heavy chains were then combined to generate a total of fifteen antibodies, named herein as Antibody A to Antibody O, and provided in Table 10 below. The amino acid sequences of the light chain and heavy chain in Antibody A to Antibody O are provided in Table 4.

TABLE 10
Anti-IL-11 Antibody Light and Heavy Chain Combinations

| Antibody | Light Chain | Heavy Chain |
|---|---|---|
| A | IgK wild type | IgG1 VH wild type |
| | (parental) | (parental) |
| B | IgK D59E variant | IgG1 VH wild type |
| | | (parental) |
| C | IgK D59A variant | IgG1 VH wild type |
| | | (parental) |
| D | IgK wild type | IgG1 D31S variant |
| | (parental) | |
| E | IgK D59E variant | IgG1 D31S variant |
| F | IgK D59A variant | IgG1 D31S variant |
| G | IgK wild type | IgG1 N55G variant |
| | (parental) | |
| H | IgK D59E variant | IgG1 N55G variant |
| I | IgK D59A variant | IgG1 N55G variant |
| J | IgK wild type | IgG1 N55Q variant |
| | (parental) | |
| K | IgK D59E variant | IgG1 N55Q variant |
| L | IgK D59A variant | IgG1 N55Q variant |
| M | IgK wild type | IgG1 N55S variant |
| | (parental) | |
| N | IgK D59E variant | IgG1 N55S variant |
| O | IgK D59A variant | IgG1 N55S variant |

### Example 2. Binding Affinities of Anti-IL-11 Antibodies

The binding affinities of Antibody A to Antibody O for IL-11 were determined by surface plasmon resonance (SPR) using a ProteOn XPR36 (Bio-Rad). Briefly, the running buffer and all dilutions were performed in PBS-T-EDTA with 0.01% Tween 20 (100 μl of 100% Tween 20 is added to 2 L of PBS-T-EDTA to make final Tween 20 concentration of 0.01%). The GLM sensorchip was then normalized and pre-conditioned as per the manufacturer's recommendations. Next, the sensorchip was activated with an equal mixture of EDC/s-NHS in the horizontal direction for 300 seconds at a flow rate of 30 μl/minute and immobilized with Protein A/G (60 μg/mL in 10 mM acetate pH 4.5) in the horizontal direction for 300 seconds at a flowrate of 30 μl/minute resulting in ~5000-5300 RU of Protein A/G on the surface. The sensorchip was then deactivated with 1M ethanolamine HCl in the horizontal direction for 300 seconds at a flowrate of 30 μl/minute. Next, the sensorchip was stabilized for 18 seconds with 0.85% phosphoric acid at a flowrate of 100 μl/minute 3 times horizontally and 3 times vertically.

Each antibody (1 μg/mL) was then captured on the Protein A/G surface vertically for 70 seconds at a flowrate of 30 μl/min resulting in capture levels of ~600-800 RU. The baseline was stabilized by injecting PBS-T-EDTA for 60 seconds at a flowrate of 100 μl/min horizontally and then a second injection of PBS-T-EDTA for 60 seconds at a flowrate of 100 μl/min and a dissociation 120 seconds horizontally. The analyte (human IL-11, cyno IL-11, or mouse IL-11) was injected horizontally over the captured antibody for 600 seconds at a flowrate of 30 μl/min and a dissociation for 1200 seconds. The concentrations of the human IL-11 and mouse IL-11 were 0 nM, 0.625 nM, 1.25 nM, 2.5 nM, 5 nM, and 10 nM. The concentrations of the cyno IL-11 were 0 nM, 31.25 nM, 62.5 nM, 125 nM, 250 nM, and 500 nM. The surface was regenerated by injecting 0.85% phosphoric acid for 18 seconds at a flowrate of 100 μl/min one time horizontally and one time vertically. PBS-T-EDTA was injected for 60 seconds at a flowrate of 100 mL/min one time vertically.

The interspot (interactions with sensor surface) and blank (PBS-T-EDTA with 0.01% Tween 20 or 0 nM analyte) were subtracted from the raw data. Sensorgrams were then fit globally to 1:1 Langmuir binding to provide on-rate (ka), off-rate (kd), and affinity (KD) values or equilibrium binding (KD).

The results for binding affinities of anti-IL-11 antibodies are shown in Tables 11 to 13 below. Antibody N is cross reactive to human IL-11 (hIL-11), mouse IL-11, marmoset IL-11, and rat IL-11. It binds to hIL-11 with a KD of 0.96±0.03 nM, mouse IL-11 with a KD of 1.74±0.02 nM, marmoset IL-11 with a KD of 1.14 nM, and rat IL-11-Fc with a KD of 0.79 nM. However, Antibody N has a low binding affinity to cyno IL-11 with a KD of 120.4±3.3 nM which may be explained by differences in primary sequences within the IL-11 binding epitope.

TABLE 11
Binding Affinity of Anti-IL-11 Antibodies (Antibody
A to Antibody O) to Human IL-11 Protein

| Antibody | | | | |
|---|---|---|---|---|
| Name | kₐ (1/Ms) | k_{d} (1/s) | K_{D} (M) | K_{D} (nM) |
| Antibody A | 1.04E+06 | 6.10E−04 | 5.88E−10 | 0.59 |
| Antibody B | 1.07E+06 | 5.32E−04 | 4.97E−10 | 0.50 |
| Antibody C | 1.28E+06 | 9.40E−04 | 7.35E−10 | 0.74 |
| Antibody D | 9.49E+05 | 1.00E−03 | 1.06E−09 | 1.06 |
| Antibody E | 1.21E+06 | 8.71E−04 | 7.18E−10 | 0.72 |
| Antibody F | 9.95E+05 | 1.30E−03 | 1.30E−09 | 1.30 |
| Antibody G | 1.02E+06 | 8.77E−04 | 8.57E−10 | 0.86 |
| Antibody H | 1.04E+06 | 7.57E−04 | 7.26E−10 | 0.73 |
| Antibody I | 1.27E+06 | 1.24E−03 | 9.77E−10 | 0.98 |
| Antibody J | 8.32E+05 | 7.58E−04 | 9.11E−10 | 0.91 |
| Antibody K | 1.16E+06 | 7.49E−04 | 6.44E−10 | 0.64 |
| Antibody L | 8.27E+05 | 1.03E−03 | 1.25E−09 | 1.25 |
| Antibody M | 1.06E+06 | 6.48E−04 | 6.12E−10 | 0.61 |
| Antibody N | 1.12E+06 | 5.21E−04 | 4.63E−10 | 0.46 |
| Antibody O | 1.33E+06 | 1.01E−03 | 7.59E−10 | 0.76 |

TABLE 12
Binding Affinity of Anti-IL-11 Antibodies (Antibody
A to Antibody O) to Cyno IL-11 Protein

| Antibody | | |
|---|---|---|
| Name | K_{D} (M) | K_{D} (nM) |
| Antibody A | 4.12E−08 | 41.2 |
| Antibody B | 3.24E−08 | 32.4 |
| Antibody C | 3.97E−08 | 39.7 |
| Antibody D | 5.71E−08 | 57.1 |
| Antibody E | 9.92E−08 | 99.2 |
| Antibody F | 6.05E−08 | 60.5 |
| Antibody G | 5.28E−08 | 52.8 |
| Antibody H | 4.49E−08 | 44.9 |
| Antibody I | 5.16E−08 | 51.6 |
| Antibody J | 5.78E−08 | 57.8 |
| Antibody K | 9.37E−08 | 93.7 |
| Antibody L | 6.14E−08 | 61.4 |
| Antibody M | 3.79E−08 | 37.9 |
| Antibody N | 3.25E−08 | 32.5 |
| Antibody O | 4.37E−08 | 43.7 |

TABLE 13
Binding Affinity of Anti-IL-11 Antibodies (Antibody
A to Antibody O) to Mouse IL-11 Protein

| Antibody | | | | |
|---|---|---|---|---|
| Name | kₐ (1/Ms) | k_{d} (1/s) | K_{D} (M) | K_{D} (nM) |
| Antibody A | 1.34E+06 | 8.87E−04 | 6.60E−10 | 0.66 |
| Antibody B | 1.46E+06 | 7.87E−04 | 5.40E−10 | 0.54 |
| Antibody C | 1.77E+06 | 1.32E−03 | 7.45E−10 | 0.75 |
| Antibody D | 1.34E+06 | 1.41E−03 | 1.05E−09 | 1.05 |
| Antibody E | 1.68E+06 | 1.09E−03 | 6.49E−10 | 0.65 |
| Antibody F | 1.32E+06 | 1.96E−03 | 1.49E−09 | 1.49 |
| Antibody G | 1.41E+06 | 1.43E−03 | 1.02E−09 | 1.02 |
| Antibody H | 1.45E+06 | 1.26E−03 | 8.70E−10 | 0.87 |
| Antibody I | 1.80E+06 | 1.98E−03 | 1.10E−09 | 1.10 |
| Antibody J | 1.18E+06 | 1.25E−03 | 1.06E−09 | 1.06 |
| Antibody K | 1.64E+06 | 1.19E−03 | 7.25E−10 | 0.73 |
| Antibody L | 1.13E+06 | 1.57E−03 | 1.39E−09 | 1.39 |
| Antibody M | 1.40E+06 | 1.26E−03 | 8.97E−10 | 0.90 |
| Antibody N | 1.41E+06 | 9.81E−04 | 6.97E−10 | 0.70 |
| Antibody O | 1.76E+06 | 1.65E−03 | 9.35E−10 | 0.94 |

### Example 3. Anti-IL-11 Antibodies Block IL-11 Signaling

Antibody A to Antibody O were assessed to determine if they are capable of blocking IL-11 signaling. Briefly, Antibody A to Antibody O were tested in a phospho-STAT3 assay in IL-11-stimulated human lung carcinoma cells (HTB183 cells) with endogenous IL-11Rα. Antibody A to Antibody O dose-dependently reduced p-STAT3 in 50 ng/ml (2.5 nM) of IL-11-stimulated HTB183 cells. The average IC₅₀ value of Antibody N is 4.04 nM as shown in Table 14.

TABLE 14
Summary of IC₅₀ Values of IL-11 Neutralizing Antibodies
in the IL-11 Stimulated HTB183 p-STAT3 Inhibition Assay

| | IC₅₀ In HTB183 |
|---|---|
| | p-STAT3 Assay |
| | (Average of |
| Antibody | several replicates) |
| Antibody B | 2.95E−09 |
| Antibody C | 6.2E−09 |
| Antibody D | 1.27E−08 |
| Antibody E | 9.34E−09 |
| Antibody F | 1.22E−08 |
| Antibody G | 7.77E−09 |
| Antibody H | 7.6E−09 |
| Antibody I | 8.99E−09 |
| Antibody J | 1.26E−08 |
| Antibody K | 8.89E−09 |
| Antibody L | 1.65E−08 |
| Antibody M | 6.99E−09 |
| Antibody N | 4.04E−09 |
| Antibody O | 6.75E−09 |
| Antibody A | 6.46E−09 |

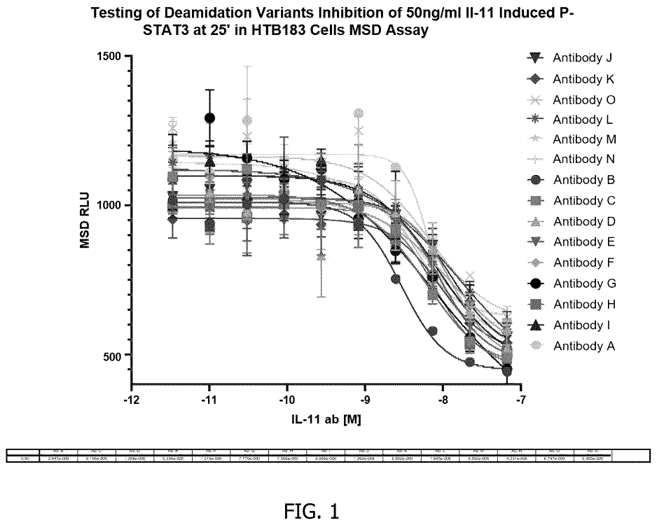

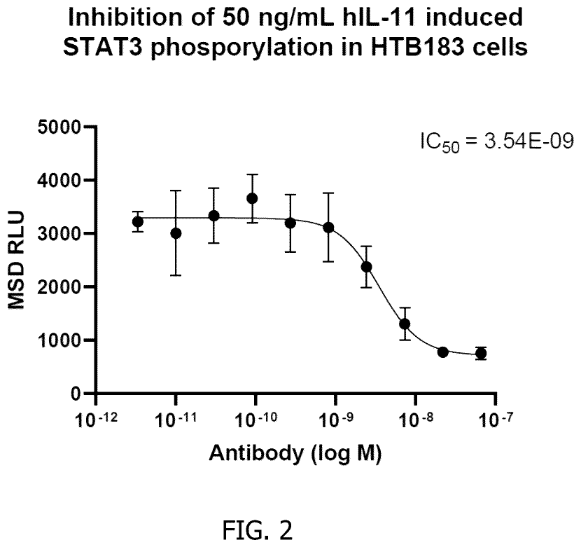

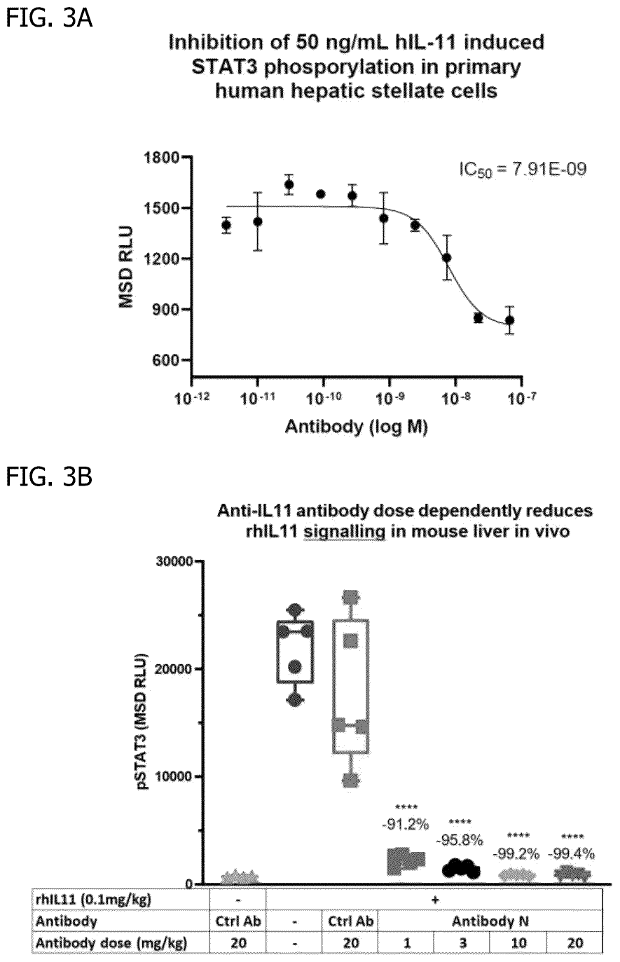

Antibody N also blocked IL-11 signaling as evidenced by inhibition of recombinant IL-11 stimulated p-STAT3 phosphorylation in HTB183 cells (FIG. 1 and FIG. 2), primary human hepatic stellate cells (HHSC) (FIG. 3A), and mouse liver (FIG. 3B). Additionally, Antibody N inhibited recombinant rat IL-11 (cis signaling) in primary rat hepatic stellate cells as well as recombinant marmoset IL-11 (cis signaling) in primary human cardiac atrial fibroblasts (See, Table 15).

TABLE 15
Summary of IC₅₀ values of Antibody N in the
IL-11 Stimulated p-STAT3 Inhibition Assay

| | IC₅₀ p-STAT3 Assay |
|---|---|
| Cell Type | (Average of replicates) |
| Primary Human Cardiac | 1.42E−08 |
| Fibroblast Cells (cis | |
| signaling) | |
| Primary Rat Hepatic | 2.79E−10 |
| Stellate Cell | |

### Example 4. Thermostability of Anti-IL-11 Antibodies

Thermal stability profiles were acquired using a QuantStudio 5 Flex real-time PCR system (Applied Biosystems) with SYPRO Orange (Invitrogen) as the extrinsic fluorophore. Briefly, each sample was diluted to approximately 0.4 g/L in 10 mM histidine, pH 6.0 with 20 mM sodium chloride and 5× (final) SYPRO Orange. Melt curves were generated using a thermal ramp from 25 to 95° C. at a rate of 2° C./min, with data collected approximately every 0.4° C. through Ex 1 (λ=470±15 nm) and Em3 (λ=623±10 nm) excitation and emission filters, respectively. Raw fluorescent data were transformed and analyzed to detect inflection points in the first derivative of fluorescent data (Tm); plots were then visually inspected to confirm reported values for Fc (CH2) and Fab/CH3. As shown in Table 16 below, Antibody N had a higher Fab melting temperature than Antibody A.

TABLE 16
Melting Temperatures of Antibody A and Antibody N

Tm (° C.)

| | Fc | Fab |
|---|---|---|
| Antibody A | 66.8 | 82.6 |
| Antibody N | 66.8 | 85.9 |

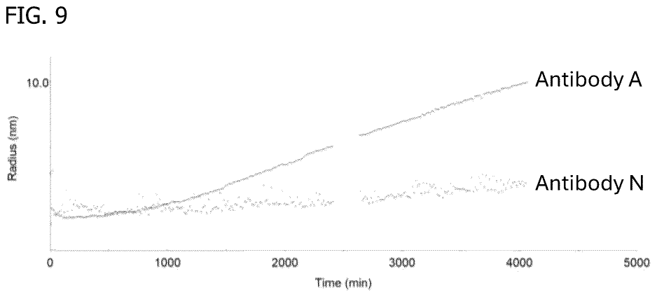

Isothermal stability was measured over approximately 3 days using a Dynamic Light Scattering (DLS) plate reader. Samples at 1 mg/ml in 10 mM histidine, pH 6.0 with 20 mM sodium chloride were held at 64° C. with measurements acquired every 20 minutes. These data were analyzed with the Cumulants method and visualized as a plot of radius versus time. Antibody N exhibited a smaller change in radius over time compared to Antibody A, indicating greater isothermal stability (FIG. 9).

### Example 5. Long-Term Stability of Anti-IL-11 Antibodies

The stability of Antibody N drug product was monitored at the proposed long-term storage condition (e.g. 5±3° C.) and in 2 mL glass vials with rubber stopper and crip cap, which is identical to the container closure system intended for the clinical drug product.

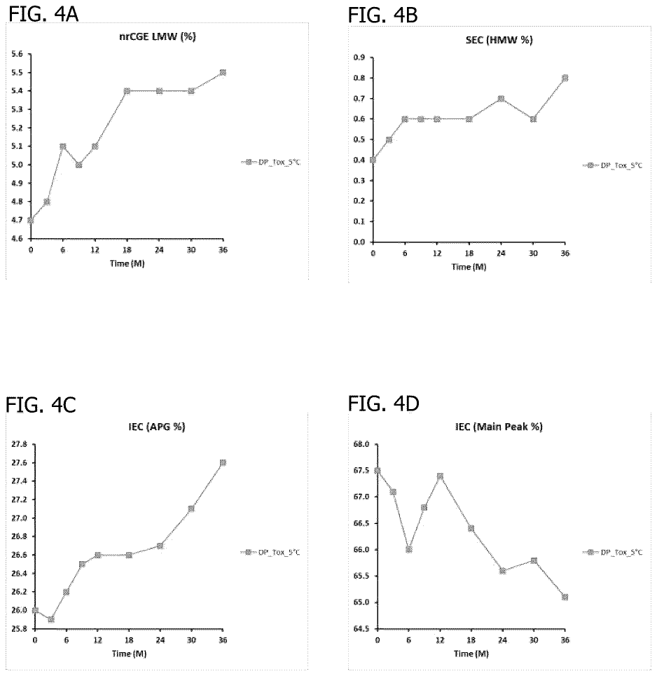

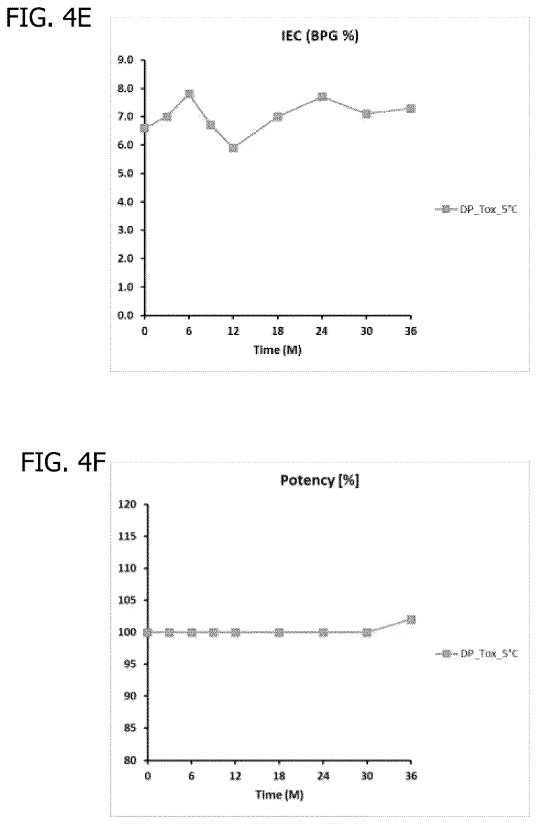

To date, 36 months real stability data are available and no significant changes were observed for any test parameter, most notably the stability indicating parameters including purity (low molecular weight species (LMW) (FIG. 4A) monitored by non-reduced capillary gel electrophoresis (CGE) as well as high molecular weight species (HMW) (FIG. 4B) monitored by size exclusion chromatography (SEC)), heterogeneity (acidic peak group (APG) (FIG. 4C), main peak (MP) (FIG. 4D), basic peak group (BPG) (FIG. 4E) by ion exchange chromatography (IEC), and potency (FIG. 4F).

### Example 6. Blocking of IL-11 Stimulates TGFβ1 HHSC Activation

### Scar-In-a-Jar Assay

Primary human hepatic stellate cells (HSC, ScienCell, Cat #5300) were plated into growth medium in PerkinElmer CellCarrier-96 plates containing 5000 cells per well. The next day, cells are washed two times and incubated in starvation medium (no FBS) for 24 hours at 37° C. with 5% CO2. Anti-IL11 antibodies or isotype control (anti-DIG higG1K, generated by Boehringer Ingelheim Pharmaceutical, Inc.) are diluted in medium containing 1% FBS and added to serum starved HSC. After 40 minutes, 10× concentrated human TGFβ1 is added to each well, for a final concentration of 5 ng/ml and cells are incubated at 37° C. for 72 hours. Supernatants are removed and cells are fixed, permeabilized and blocked prior to immunohistochemical staining and imaging. Anti-αSMA (Abcam Catalog #ab7817, AlexFluor-555; 1:100) and anti-fibronectin (Abcam Catalog #198934, AlexaFluor-647, 1:100) antibodies are incubated on the plate overnight with shaking at 400 rpm in 3% BSA/PBS. Cells are washed with PBS/0.02% TritonX100, stained with Hoechst 33342, washed again and sealed with light blocking foil. All primary analysis is done on Cellinsight CX7 and analyzed in HCS Studio, 6.6.1. % HIGH_ObjectSpotTotalInten is defined as 2STD above the mean of a control (no TGFβ) well, and data are exported to Microsoft Excel. In some cases, MMP2 is measured in the cell supernatants after 24 hrs treatment utilizing human MMP2 antibody set (MSD R-Plex Cat #F218C-8).

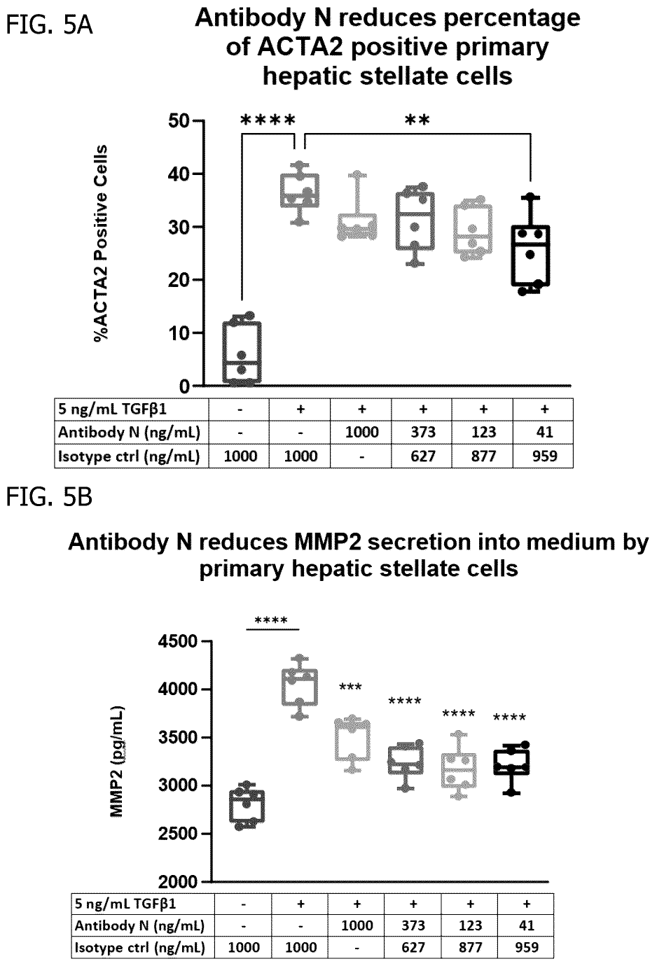

Antibody N was tested for its ability to reduce markers of liver fibrosis in HHSCs stimulated with TGFβ1. Antibody N reduced the percent of liver fibrosis marker (ACTA2) activation in HHSCs stimulated with 5 ng/ml of TGFβ1 (FIG. 5A). It also inhibited activation markers alpha-smooth muscle actin (α-SMA), FN1 and COL3A1 in the scar-in-a-jar assay, and reduced MMP2 and procollagen secretion at various concentrations of TGFβ1 stimulated HHSCs ranging from 41 ng/ml to 10 μg/mL, which corresponds to 0.27-67 nM (FIG. 5B).

### Example 7. Assessment of Anti-IL-11 Antibody N in A Mouse Bleomycin Model

Non-clinical pharmacology studies aimed at exploring the efficacy of Antibody N in pulmonary fibrosis will be employed in two in vivo model systems including, a mechanistic model of IL11 instillation into the lungs of mice and a modified aged murine bleomycin mouse model of lung fibrosis. Briefly, in the IL11 instillation model, p-STAT3 signalling will be assessed in lung tissue 15 minutes following delivery of IL11. An Antibody P injection will be administered 24 hours prior to IL11 change followed by an assessment of p-STAT3. Additionally, IL-11 expression will be measured in a comparison of young (8-10 weeks) vs. aged (18 months) mice responding to bleomycin injury. In this model, the efficacy of Antibody P, a murine cross-reactive tool closely related to Antibody N, will be tested therapeutically, dosed in the repair phase from Days 21-42 at a weight-adjusted dose of 16 mg/kg (versus isotype control at the same dose). Assessments may include the measurement of inflammatory cell content (e.g., macrophages and lymphocytes) of bronchoalveolar lavage (BAL) fluid, measurement of recovery of lung function (e.g., as assessed by compliance and pressure/volume loops), measurement of the release of surfactant protein D (SP-D) into the blood, and the determination of a fibrosis score.

### Example 8. Antibody N Reduces Fibrosis in a Murine Model of Liver Fibrosis

The efficacy of Antibody N was assessed in high fat methionine choline deficient (HFMCD) mice, a steatosis driven mouse liver fibrosis model. Briefly, mice were fed the HFMCD (with 0.1% methionine) diet for 6 weeks to induce liver steatosis and fibrosis. Pharmacological treatment was then started one week after the initiation of the HFMCD diet and continued for 5 weeks.

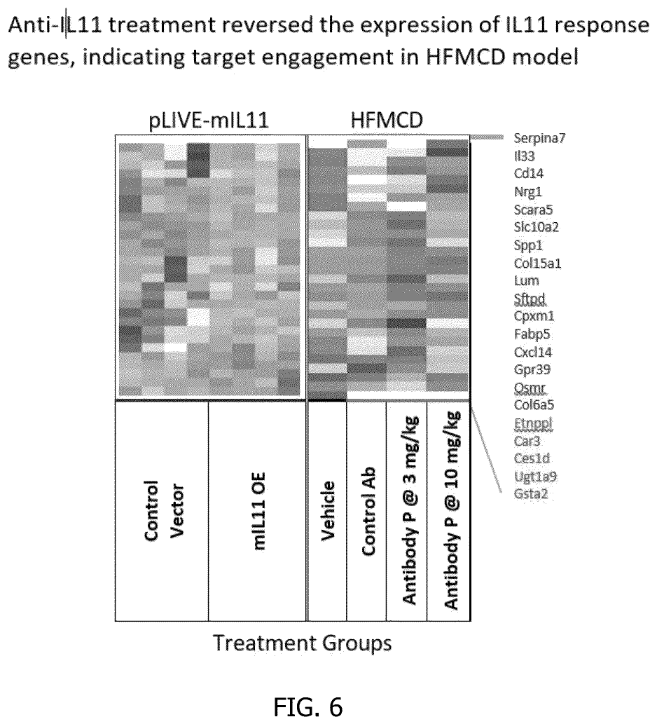

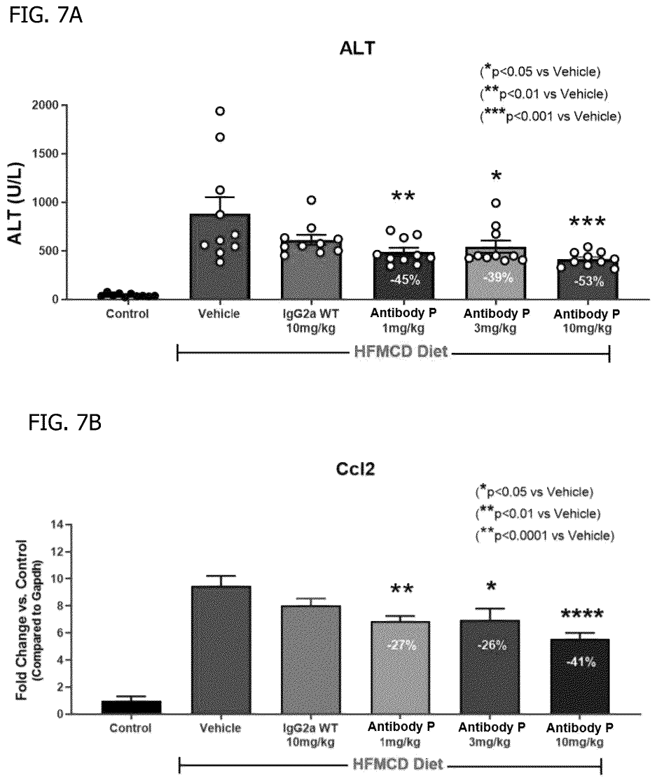

To test the efficacy of Antibody N in the mouse model of liver fibrosis and avoid an immune response against a human antibody, the human IgG1 Fc in Antibody N was replaced with mouse IgG2a Fc to generate a mouse IgG version of the antibody (Antibody P). The effects of Antibody P on hepatocyte injury, liver inflammation and liver fibrosis are shown in FIGS. 6-8. Liver expression of IL-11 signature genes are profoundly modulated, indicating blockade of IL-11 signaling in the mouse liver (FIG. 6). The anti-IL-11 antibody elicited significant reductions in serum liver injury marker ALT (FIG. 7A) at all doses tested, indicative of a reduction in hepatocyte injury. Anti-IL-11 antibody treatment also led to a dose-dependent reduction in Ccl2 mRNA (FIG. 7B), a marker of inflammation, supporting a reduction of liver inflammation. These results differentiate Antibody P from the anti-fibrotic positive control compound Alk5 inhibitor, which had no effect on reducing hepatocyte injury and liver inflammation.

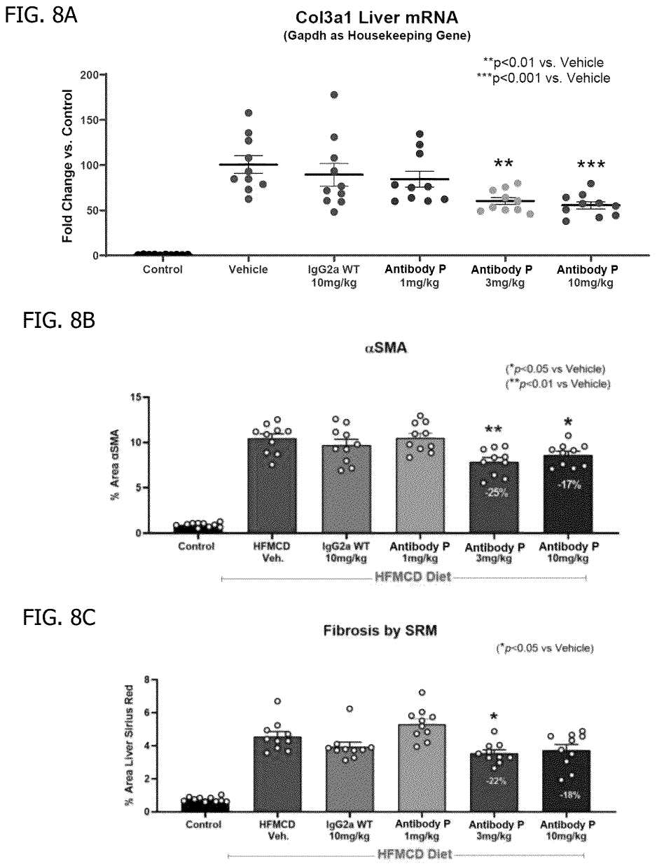

Antibody P also reduced liver fibrosis shown by a dose-dependent reduction in type III collagen (COL3A1) mRNA, a fibrosis marker (FIG. 8A). Antibody P also significantly reduced HSC activation as measured by the percent positive area of α-SMA, a marker of HSC activation (FIG. 8B) and sirius red morphometry (SRM), a liver fibrosis marker (FIG. 8C), at 3 mg/kg and 10 mg/kg doses versus vehicle. A dose-dependent modulation of Col3a1 mRNA and α-SMA positively correlated with liver fibrosis measured by SRM (FIGS. 8A-8C).

Specific embodiments provided herein can be further limited in the claims using “consisting of” or “consisting essentially of” language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments so claimed are inherently or expressly described and enabled herein.

In cases where numerical values are indicated herein, the skilled person will understand that the technical effect of the feature in question is ensured within an interval of accuracy, which typically encompasses a deviation of the numerical value given of ±10% or of ±5%. At the very least, and not as an attempt to limit the application of the doctrine of equivalents to the scope of the claims, each numerical parameter should at least be construed considering the number of reported significant digits and by applying ordinary rounding techniques.

Unless otherwise indicated, all numbers expressing quantities of ingredients, properties such as molecular weight and median size, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained.

The terms “a,” “an,” “the” and similar referents used in the context of the description herein (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples or exemplary language (e.g., “such as”) provided herein is intended merely to better illuminate the specification and does not pose a limitation on the scope of the claims. No language in the specification should be construed as indicating any non-claimed element essential to the practice of the description.

Unless specifically stated or obvious from context, as used herein, the term “or” is understood to be inclusive and covers both “or” and “and.”

Groupings of alternative elements or embodiments provided herein are not to be construed as limitations. Each group member can be referred to and claimed individually or in any combination with other members of the group or other elements found herein. It is anticipated that one or more members of a group can be included in, or deleted from, a group for reasons of convenience and/or patentability. When any such inclusion or deletion occurs, the specification is deemed to contain the group as modified, thus fulfilling the written description of all Markush groups used in the appended claims.

Certain embodiments are described herein, including the best mode known for carrying out methods provided herein. Of course, variations on these described embodiments will become apparent upon reading the foregoing description. One can be expected to employ such variations as appropriate and can be practiced other than as specifically described herein. Accordingly, this description includes all modifications and equivalents of the subject matter recited in the claims appended hereto as permitted by applicable law. Moreover, any combination of the above-described elements in all possible variations thereof is encompassed by the description unless otherwise indicated herein or otherwise clearly contradicted by context.

It is to be understood that the embodiments provided herein are illustrative of the principles of the description herein. Other modifications that can be employed are within the scope of the description. Thus, by way of example, but not of limitation, alternative configurations can be utilized in accordance with the teachings herein. Accordingly, the presented information is not limited to that precisely as shown and described.

While the present description has been described and illustrated herein by references to various specific materials, procedures, and examples, it is understood that the description is not restricted to the particular combinations of materials and procedures selected for that purpose. Numerous variations of such details can be implied as will be appreciated. It is intended that the specification and examples be considered as exemplary only, with the true scope and spirit of the specification being indicated by the following claims. All references, patents, and patent applications referred to in this application are herein incorporated by reference in their entirety.

Unless defined otherwise, all technical and scientific terms used herein have the same meaning as commonly understood. Although other probes, compositions, methods, and kits similar, or equivalent, to those described herein can be used in the practice described herein, the materials and methods are described herein. It is to be understood that the terminology used herein is for the purpose of describing embodiments only and is not intended to be limiting.

Unless specifically stated or obvious from context, as used herein, the term “about” is understood as within a range of normal tolerance, for example within 2 standard deviations of the mean. About is understood to be within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

A stated range is understood to be any value between and at the limits of the stated range. As examples, a range between 1 and 5 includes 1, 2, 3, 4, and 5; a range between 1 and 10 includes 1, 2, 3, 4, 5, 6, 7, 8, 9, and 10; and a range between 1 and 100 includes 1, 2, 3, 4, 5, 6, 7, 8, 9, 10, 11, 12, 13, 14, 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, and 100.

Any aspect or embodiment described herein can be combined with any other aspect or embodiment as described herein.

1-86. (canceled)

87. An anti-interleukin-11 (IL-11) antibody or an antigen-binding fragment thereof comprising:
a heavy chain variable region (VH) comprising the amino acid sequence of SEQ ID NO: 1 (H-CDR1) or SEQ ID NO: 2 (H-CDR1), the amino acid sequence of SEQ ID NO: 3 (H-CDR2), SEQ ID NO: 4 (H-CDR2), SEQ ID NO: 5 (H-CDR2), or SEQ ID NO: 6 (H-CDR2), and the amino acid sequence of SEQ ID NO: 7 (H-CDR3); and
a light chain variable region (VL) comprising the amino acid sequence of SEQ ID NO: 9 (L-CDR1), the amino acid sequence of SEQ ID NO: 10 (L-CDR2), SEQ ID NO: 11 (L-CDR2), or SEQ ID NO: 12 (L-CDR2) and the amino acid sequence of SEQ ID NO: 13 (L-CDR3),
with the proviso that if the VH comprises the amino acid sequence of SEQ ID NO:1 (H-CDR1), the amino acid sequence of SEQ ID NO: 4 (H-CDR2), and the amino acid sequence of SEQ ID NO: 7 (H-CDR3), then the VL comprises the amino acid sequence of SEQ ID NO: 10 (L-CDR2) or SEQ ID NO: 12 (L-CDR2).

88. The anti-IL-11 antibody or antigen-binding fragment thereof according to claim 87, wherein the antibody or an antigen-binding fragment thereof comprises:
i. a heavy chain variable region (VH) comprising the amino acid sequence of SEQ ID NO: 1 (H-CDR1), the amino acid sequence of SEQ ID NO: 4 (H-CDR2), and the amino acid sequence of SEQ ID NO: 7 (H-CDR3); and a light chain variable region (VL) comprising the amino acid sequence of SEQ ID NO: 9 (L-CDR1), the amino acid sequence of SEQ ID NO: 10 (L-CDR2), and the amino acid sequence of SEQ ID NO: 13 (L-CDR3); or
ii. a heavy chain variable region (VH) comprising the amino acid sequence of SEQ ID NO: 1 (H-CDR1), the amino acid sequence of SEQ ID NO: 4 (H-CDR2), and the amino acid sequence of SEQ ID NO: 7 (H-CDR3); and a light chain variable region (VL) comprising the amino acid sequence of SEQ ID NO: 9 (L-CDR1), the amino acid sequence of SEQ ID NO: 12 (L-CDR2), and the amino acid sequence of SEQ ID NO: 13 (L-CDR3); or
iii. a heavy chain variable region (VH) comprising the amino acid sequence of SEQ ID NO: 2 (H-CDR1), the amino acid sequence of SEQ ID NO: 4 (H-CDR2), and the amino acid sequence of SEQ ID NO: 7 (H-CDR3); and a light chain variable region (VL) comprising the amino acid sequence of SEQ ID NO: 9 (L-CDR1), the amino acid sequence of SEQ ID NO: 11 (L-CDR2), and the amino acid sequence of SEQ ID NO: 13 (L-CDR3); or
iv. a heavy chain variable region (VH) comprising the amino acid sequence of SEQ ID NO: 2 (H-CDR1), the amino acid sequence of SEQ ID NO: 4 (H-CDR2), and the amino acid sequence of SEQ ID NO: 7 (H-CDR3); and a light chain variable region (VL) comprising the amino acid sequence of SEQ ID NO: 9 (L-CDR1), the amino acid sequence of SEQ ID NO: 10 (L-CDR2), and the amino acid sequence of SEQ ID NO: 13 (L-CDR3); or
v. a heavy chain variable region (VH) comprising the amino acid sequence of SEQ ID NO: 2 (H-CDR1), the amino acid sequence of SEQ ID NO: 4 (H-CDR2), and the amino acid sequence of SEQ ID NO: 7 (H-CDR3); and a light chain variable region (VL) comprising the amino acid sequence of SEQ ID NO: 9 (L-CDR1), the amino acid sequence of SEQ ID NO: 12 (L-CDR2), and the amino acid sequence of SEQ ID NO: 13 (L-CDR3); or
vi. a heavy chain variable region (VH) comprising the amino acid sequence of SEQ ID NO: 1 (H-CDR1), the amino acid sequence of SEQ ID NO: 5 (H-CDR2), and the amino acid sequence of SEQ ID NO: 7 (H-CDR3); and a light chain variable region (VL) comprising the amino acid sequence of SEQ ID NO: 9 (L-CDR1), the amino acid sequence of SEQ ID NO: 11 (L-CDR2), and the amino acid sequence of SEQ ID NO: 13 (L-CDR3); or
vii. a heavy chain variable region (VH) comprising the amino acid sequence of SEQ ID NO: 1 (H-CDR1), the amino acid sequence of SEQ ID NO: 5 (H-CDR2), and the amino acid sequence of SEQ ID NO: 7 (H-CDR3); and a light chain variable region (VL) comprising the amino acid sequence of SEQ ID NO: 9 (L-CDR1), the amino acid sequence of SEQ ID NO: 10 (L-CDR2), and the amino acid sequence of SEQ ID NO: 13 (L-CDR3); or
viii. a heavy chain variable region (VH) comprising the amino acid sequence of SEQ ID NO: 1 (H-CDR1), the amino acid sequence of SEQ ID NO: 5 (H-CDR2), and the amino acid sequence of SEQ ID NO: 7 (H-CDR3); and a light chain variable region (VL) comprising the amino acid sequence of SEQ ID NO: 9 (L-CDR1), the amino acid sequence of SEQ ID NO: 12 (L-CDR2), and the amino acid sequence of SEQ ID NO: 13 (L-CDR3); or
ix. a heavy chain variable region (VH) comprising the amino acid sequence of SEQ ID NO: 1 (H-CDR1), the amino acid sequence of SEQ ID NO: 6 (H-CDR2), and the amino acid sequence of SEQ ID NO: 7 (H-CDR3); and a light chain variable region (VL) comprising the amino acid sequence of SEQ ID NO: 9 (L-CDR1), the amino acid sequence of SEQ ID NO: 11 (L-CDR2), and the amino acid sequence of SEQ ID NO: 13 (L-CDR3); or
x. a heavy chain variable region (VH) comprising the amino acid sequence of SEQ ID NO: 1 (H-CDR1), the amino acid sequence of SEQ ID NO: 6 (H-CDR2), and the amino acid sequence of SEQ ID NO: 7 (H-CDR3); and a light chain variable region (VL) comprising the amino acid sequence of SEQ ID NO: 9 (L-CDR1), the amino acid sequence of SEQ ID NO: 10 (L-CDR2), and the amino acid sequence of SEQ ID NO: 13 (L-CDR3); or
xi. a heavy chain variable region (VH) comprising the amino acid sequence of SEQ ID NO: 1 (H-CDR1), the amino acid sequence of SEQ ID NO: 6 (H-CDR2), and the amino acid sequence of SEQ ID NO: 7 (H-CDR3); and a light chain variable region (VL) comprising the amino acid sequence of SEQ ID NO: 9 (L-CDR1), the amino acid sequence of SEQ ID NO: 12 (L-CDR2), and the amino acid sequence of SEQ ID NO: 13 (L-CDR3); or
xii. a heavy chain variable region (VH) comprising the amino acid sequence of SEQ ID NO: 1 (H-CDR1), the amino acid sequence of SEQ ID NO: 3 (H-CDR2), and the amino acid sequence of SEQ ID NO: 7 (H-CDR3); and a light chain variable region (VL) comprising the amino acid sequence of SEQ ID NO: 9 (L-CDR1), the amino acid sequence of SEQ ID NO: 11 (L-CDR2), and the amino acid sequence of SEQ ID NO: 13 (L-CDR3); or
xiii. a heavy chain variable region (VH) comprising the amino acid sequence of SEQ ID NO: 1 (H-CDR1), the amino acid sequence of SEQ ID NO: 3 (H-CDR2), and the amino acid sequence of SEQ ID NO: 7 (H-CDR3); and a light chain variable region (VL) comprising the amino acid sequence of SEQ ID NO: 9 (L-CDR1), the amino acid sequence of SEQ ID NO: 10 (L-CDR2), and the amino acid sequence of SEQ ID NO: 13 (L-CDR3); or
xiv. a heavy chain variable region (VH) comprising the amino acid sequence of SEQ ID NO: 1 (H-CDR1), the amino acid sequence of SEQ ID NO: 3 (H-CDR2), and the amino acid sequence of SEQ ID NO: 7 (H-CDR3); and a light chain variable region (VL) comprising the amino acid sequence of SEQ ID NO: 9 (L-CDR1), the amino acid sequence of SEQ ID NO: 12 (L-CDR2), and the amino acid sequence of SEQ ID NO: 13 (L-CDR3).

89. The anti-IL-11 antibody or antigen-binding fragment thereof according to claim 87, wherein the antibody or antigen-binding fragment thereof comprises a VH that has at least about 80% sequence identity to the amino acid sequence of SEQ ID NO: 14, 15, 16, 17, or 18; and a VL that has at least about 80% sequence identity to the amino acid sequence of SEQ ID NO: 19, 20, or 21.

90. The anti-IL-11 antibody or antigen-binding fragment thereof according to claim 87, wherein the antibody or antigen-binding fragment thereof comprises:
i. a VH that has at least about 80% sequence identity to the amino acid sequence of SEQ ID NO: 15; and a VL that has at least about 80% sequence identity to the amino acid sequence of SEQ ID NO: 19; or
ii. a VH that has at least about 80% sequence identity to the amino acid sequence of SEQ ID NO: 15; and a VL that has at least about 80% sequence identity to the amino acid sequence of SEQ ID NO: 21; or
iii. a VH that has at least about 80% sequence identity to the amino acid sequence of SEQ ID NO: 16; and a VL that has at least about 80% sequence identity to the amino acid sequence of SEQ ID NO: 20; or
iv. a VH that has at least about 80% sequence identity to the amino acid sequence of SEQ ID NO: 16; and a VL that has at least about 80% sequence identity to the amino acid sequence of SEQ ID NO: 19; or
v. a VH that has at least about 80% sequence identity to the amino acid sequence of SEQ ID NO: 16; and a VL that has at least about 80% sequence identity to the amino acid sequence of SEQ ID NO: 21; or
vi. a VH that has at least about 80% sequence identity to the amino acid sequence of SEQ ID NO: 17; and a VL that has at least about 80% sequence identity to the amino acid sequence of SEQ ID NO: 20; or
vii. a VH that has at least about 80% sequence identity to the amino acid sequence of SEQ ID NO: 17; and a VL that has at least about 80% sequence identity to the amino acid sequence of SEQ ID NO: 19; or
viii. a VH that has at least about 80% sequence identity to the amino acid sequence of SEQ ID NO: 17; and a VL that has at least about 80% sequence identity to the amino acid sequence of SEQ ID NO: 21; or
ix. a VH that has at least about 80% sequence identity to the amino acid sequence of SEQ ID NO: 18; and a VL that has at least about 80% sequence identity to the amino acid sequence of SEQ ID NO: 20; or
x. a VH that has at least about 80% sequence identity to the amino acid sequence of SEQ ID NO: 18; and a VL that has at least about 80% sequence identity to the amino acid sequence of SEQ ID NO: 19; or
xi. a VH that has at least about 80% sequence identity to the amino acid sequence of SEQ ID NO: 18; and a VL that has at least about 80% sequence identity to the amino acid sequence of SEQ ID NO: 21; or
xii. a VH that has at least about 80% sequence identity to the amino acid sequence of SEQ ID NO: 14; and a VL that has at least about 80% sequence identity to the amino acid sequence of SEQ ID NO: 20; or
xiii. a VH that has at least about 80% sequence identity to the amino acid sequence of SEQ ID NO: 14; and a VL that has at least about 80% sequence identity to the amino acid sequence of SEQ ID NO: 19; or
xiv. a VH that has at least about 80% sequence identity to the amino acid sequence of SEQ ID NO: 14; and a VL that has at least about 80% sequence identity to the amino acid sequence of SEQ ID NO: 21.

91. The anti-IL-11 antibody or antigen-binding fragment thereof according to claim 87, wherein the antibody or antigen-binding fragment thereof comprises:
i. a VH that has at least about 85% sequence identity to the amino acid sequence of SEQ ID NO: 15; and a VL that has at least about 85% sequence identity to the amino acid sequence of SEQ ID NO: 19; or
ii. a VH that has at least about 85% sequence identity to the amino acid sequence of SEQ ID NO: 15; and a VL that has at least about 85% sequence identity to the amino acid sequence of SEQ ID NO: 21; or
iii. a VH that has at least about 85% sequence identity to the amino acid sequence of SEQ ID NO: 16; and a VL that has at least about 85% sequence identity to the amino acid sequence of SEQ ID NO: 20; or
iv. a VH that has at least about 85% sequence identity to the amino acid sequence of SEQ ID NO: 16; and a VL that has at least about 85% sequence identity to the amino acid sequence of SEQ ID NO: 19; or
v. a VH that has at least about 85% sequence identity to the amino acid sequence of SEQ ID NO: 16; and a VL that has at least about 85% sequence identity to the amino acid sequence of SEQ ID NO: 21; or
vi. a VH that has at least about 85% sequence identity to the amino acid sequence of SEQ ID NO: 17; and a VL that has at least about 85% sequence identity to the amino acid sequence of SEQ ID NO: 20; or
vii. a VH that has at least about 85% sequence identity to the amino acid sequence of SEQ ID NO: 17; and a VL that has at least about 85% sequence identity to the amino acid sequence of SEQ ID NO: 19; or
viii. a VH that has at least about 85% sequence identity to the amino acid sequence of SEQ ID NO: 17; and a VL that has at least about 85% sequence identity to the amino acid sequence of SEQ ID NO: 21; or
ix. a VH that has at least about 85% sequence identity to the amino acid sequence of SEQ ID NO: 18; and a VL that has at least about 85% sequence identity to the amino acid sequence of SEQ ID NO: 20; or
x. a VH that has at least about 85% sequence identity to the amino acid sequence of SEQ ID NO: 18; and a VL that has at least about 85% sequence identity to the amino acid sequence of SEQ ID NO: 19; or
xi. a VH that has at least about 85% sequence identity to the amino acid sequence of SEQ ID NO: 18; and a VL that has at least about 85% sequence identity to the amino acid sequence of SEQ ID NO: 21; or
xii. a VH that has at least about 85% sequence identity to the amino acid sequence of SEQ ID NO: 14; and a VL that has at least about 85% sequence identity to the amino acid sequence of SEQ ID NO: 20; or
xiii. a VH that has at least about 85% sequence identity to the amino acid sequence of SEQ ID NO: 14; and a VL that has at least about 85% sequence identity to the amino acid sequence of SEQ ID NO: 19; or
xiv. a VH that has at least about 85% sequence identity to the amino acid sequence of SEQ ID NO: 14; and a VL that has at least about 85% sequence identity to the amino acid sequence of SEQ ID NO: 21.

92. The anti-IL-11 antibody or antigen-binding fragment thereof according to claim 87, wherein the antibody or antigen-binding fragment thereof comprises:
i. a VH that has at least about 90% sequence identity to the amino acid sequence of SEQ ID NO: 15; and a VL that has at least about 90% sequence identity to the amino acid sequence of SEQ ID NO: 19; or
ii. a VH that has at least about 90% sequence identity to the amino acid sequence of SEQ ID NO: 15; and a VL that has at least about 90% sequence identity to the amino acid sequence of SEQ ID NO: 21; or
iii. a VH that has at least about 90% sequence identity to the amino acid sequence of SEQ ID NO: 16; and a VL that has at least about 90% sequence identity to the amino acid sequence of SEQ ID NO: 20; or
iv. a VH that has at least about 90% sequence identity to the amino acid sequence of SEQ ID NO: 16; and a VL that has at least about 90% sequence identity to the amino acid sequence of SEQ ID NO: 19; or
v. a VH that has at least about 90% sequence identity to the amino acid sequence of SEQ ID NO: 16; and a VL that has at least about 90% sequence identity to the amino acid sequence of SEQ ID NO: 21; or
vi. a VH that has at least about 90% sequence identity to the amino acid sequence of SEQ ID NO: 17; and a VL that has at least about 90% sequence identity to the amino acid sequence of SEQ ID NO: 20; or
vii. a VH that has at least about 90% sequence identity to the amino acid sequence of SEQ ID NO: 17; and a VL that has at least about 90% sequence identity to the amino acid sequence of SEQ ID NO: 19; or
viii. a VH that has at least about 90% sequence identity to the amino acid sequence of SEQ ID NO: 17; and a VL that has at least about 90% sequence identity to the amino acid sequence of SEQ ID NO: 21; or
ix. a VH that has at least about 90% sequence identity to the amino acid sequence of SEQ ID NO: 18; and a VL that has at least about 90% sequence identity to the amino acid sequence of SEQ ID NO: 20; or
x. a VH that has at least about 90% sequence identity to the amino acid sequence of SEQ ID NO: 18; and a VL that has at least about 90% sequence identity to the amino acid sequence of SEQ ID NO: 19; or
xi. a VH that has at least about 90% sequence identity to the amino acid sequence of SEQ ID NO: 18; and a VL that has at least about 90% sequence identity to the amino acid sequence of SEQ ID NO: 21; or
xii. a VH that has at least about 90% sequence identity to the amino acid sequence of SEQ ID NO: 14; and a VL that has at least about 90% sequence identity to the amino acid sequence of SEQ ID NO: 20; or
xiii. a VH that has at least about 90% sequence identity to the amino acid sequence of SEQ ID NO: 14; and a VL that has at least about 90% sequence identity to the amino acid sequence of SEQ ID NO: 19; or
xiv. a VH that has at least about 90% sequence identity to the amino acid sequence of SEQ ID NO: 14; and a VL that has at least about 90% sequence identity to the amino acid sequence of SEQ ID NO: 21.

93. The anti-IL-11 antibody or antigen-binding fragment thereof according to claim 87, wherein the antibody or antigen-binding fragment thereof comprises:
i. a VH that has at least about 95% sequence identity to the amino acid sequence of SEQ ID NO: 15; and a VL that has at least about 95% sequence identity to the amino acid sequence of SEQ ID NO: 19; or
ii. a VH that has at least about 95% sequence identity to the amino acid sequence of SEQ ID NO: 15; and a VL that has at least about 95% sequence identity to the amino acid sequence of SEQ ID NO: 21; or
iii. a VH that has at least about 95% sequence identity to the amino acid sequence of SEQ ID NO: 16; and a VL that has at least about 95% sequence identity to the amino acid sequence of SEQ ID NO: 20; or
iv. a VH that has at least about 95% sequence identity to the amino acid sequence of SEQ ID NO: 16; and a VL that has at least about 95% sequence identity to the amino acid sequence of SEQ ID NO: 19; or
v. a VH that has at least about 95% sequence identity to the amino acid sequence of SEQ ID NO: 16; and a VL that has at least about 95% sequence identity to the amino acid sequence of SEQ ID NO: 21; or
vi. a VH that has at least about 95% sequence identity to the amino acid sequence of SEQ ID NO: 17; and a VL that has at least about 95% sequence identity to the amino acid sequence of SEQ ID NO: 20; or
vii. a VH that has at least about 95% sequence identity to the amino acid sequence of SEQ ID NO: 17; and a VL that has at least about 95% sequence identity to the amino acid sequence of SEQ ID NO: 19; or
viii. a VH that has at least about 95% sequence identity to the amino acid sequence of SEQ ID NO: 17; and a VL that has at least about 95% sequence identity to the amino acid sequence of SEQ ID NO: 21; or
ix. a VH that has at least about 95% sequence identity to the amino acid sequence of SEQ ID NO: 18; and a VL that has at least about 95% sequence identity to the amino acid sequence of SEQ ID NO: 20; or
x. a VH that has at least about 95% sequence identity to the amino acid sequence of SEQ ID NO: 18; and a VL that has at least about 95% sequence identity to the amino acid sequence of SEQ ID NO: 19; or
xi. a VH that has at least about 95% sequence identity to the amino acid sequence of SEQ ID NO: 18; and a VL that has at least about 95% sequence identity to the amino acid sequence of SEQ ID NO: 21; or
xii. a VH that has at least about 95% sequence identity to the amino acid sequence of SEQ ID NO: 14; and a VL that has at least about 95% sequence identity to the amino acid sequence of SEQ ID NO: 20; or
xiii. a VH that has at least about 95% sequence identity to the amino acid sequence of SEQ ID NO: 14; and a VL that has at least about 95% sequence identity to the amino acid sequence of SEQ ID NO: 19; or
xiv. a VH that has at least about 95% sequence identity to the amino acid sequence of SEQ ID NO: 14; and a VL that has at least about 95% sequence identity to the amino acid sequence of SEQ ID NO: 21.

94. The anti-IL-11 antibody or antigen-binding fragment thereof according to claim 87, wherein the antibody or antigen-binding fragment thereof comprises:
i. a VH comprising the amino acid sequence of SEQ ID NO: 15 and a VL comprising the amino acid sequence of SEQ ID NO: 19;
ii. a VH comprising the amino acid sequence of SEQ ID NO: 15 and a VL comprising the amino acid sequence of SEQ ID NO: 21;
iii. a VH comprising the amino acid sequence of SEQ ID NO: 16 and a VL comprising the amino acid sequence of SEQ ID NO: 20;
iv. a VH comprising the amino acid sequence of SEQ ID NO: 16 and a VL comprising the amino acid sequence of SEQ ID NO: 19;
v. a VH comprising the amino acid sequence of SEQ ID NO: 16 and a VL comprising the amino acid sequence of SEQ ID NO: 21;
vi. a VH comprising the amino acid sequence of SEQ ID NO: 17 and a VL comprising the amino acid sequence of SEQ ID NO: 20;
vii. a VH comprising the amino acid sequence of SEQ ID NO: 17 and a VL comprising the amino acid sequence of SEQ ID NO: 19;
viii. a VH comprising the amino acid sequence of SEQ ID NO: 17 and a VL comprising the amino acid sequence of SEQ ID NO: 21;
ix. a VH comprising the amino acid sequence of SEQ ID NO: 18 and a VL comprising the amino acid sequence of SEQ ID NO: 20;
x. a VH comprising the amino acid sequence of SEQ ID NO: 18 and a VL comprising the amino acid sequence of SEQ ID NO: 19;
xi. a VH comprising the amino acid sequence of SEQ ID NO: 18 and a VL comprising the amino acid sequence of SEQ ID NO: 21;
xii. a VH comprising the amino acid sequence of SEQ ID NO: 14 and a VL comprising the amino acid sequence of SEQ ID NO: 20;
xiii. a VH comprising the amino acid sequence of SEQ ID NO: 14 and a VL comprising the amino acid sequence of SEQ ID NO: 19; or
xiv. a VH comprising the amino acid sequence of SEQ ID NO: 14 and a VL comprising the amino acid sequence of SEQ ID NO: 21.

95. The anti-IL-11 antibody or antigen-binding fragment thereof according to claim 87, wherein the antibody or antigen-binding fragment thereof comprises a heavy chain comprising the amino acid sequence of SEQ ID NO: 26, 27, 28, 29, or 30; and a light chain comprising the amino acid sequence of SEQ ID NO: 31, 32, or 33.

96. The anti-IL-11 antibody or antigen-binding fragment thereof according to claim 87 comprising:
i. a heavy chain comprising the amino acid sequence of SEQ ID NO: 27, and a light chain comprising the amino acid sequence of SEQ ID NO: 31;
ii. a heavy chain comprising the amino acid sequence of SEQ ID NO: 27, and a light chain comprising the amino acid sequence of SEQ ID NO: 33;
iii. a heavy chain comprising the amino acid sequence of SEQ ID NO: 28, and a light chain comprising the amino acid sequence of SEQ ID NO: 32;
iv. a heavy chain comprising the amino acid sequence of SEQ ID NO: 28, and a light chain comprising the amino acid sequence of SEQ ID NO: 31;
v. a heavy chain comprising the amino acid sequence of SEQ ID NO: 28, and a light chain comprising the amino acid sequence of SEQ ID NO: 33;
vi. a heavy chain comprising the amino acid sequence of SEQ ID NO: 29, and a light chain comprising the amino acid sequence of SEQ ID NO: 32;
vii. a heavy chain comprising the amino acid sequence of SEQ ID NO: 29, and a light chain comprising the amino acid sequence of SEQ ID NO: 31;
viii. a heavy chain comprising the amino acid sequence of SEQ ID NO: 29, and a light chain comprising the amino acid sequence of SEQ ID NO: 33;
ix. a heavy chain comprising the amino acid sequence of SEQ ID NO: 30, and a light chain comprising the amino acid sequence of SEQ ID NO: 32;
x. a heavy chain comprising the amino acid sequence of SEQ ID NO: 30, and a light chain comprising the amino acid sequence of SEQ ID NO: 31;
xi. a heavy chain comprising the amino acid sequence of SEQ ID NO: 30, and a light chain comprising the amino acid sequence of SEQ ID NO: 33;
xii. a heavy chain comprising the amino acid sequence of SEQ ID NO: 26, and a light chain comprising the amino acid sequence of SEQ ID NO: 32;
xiii. a heavy chain comprising the amino acid sequence of SEQ ID NO: 26, and a light chain comprising the amino acid sequence of SEQ ID NO: 31; or
xiv. a heavy chain comprising the amino acid sequence of SEQ ID NO: 26, and a light chain comprising the amino acid sequence of SEQ ID NO: 33.

97. An anti-interleukin-11 (IL-11) antibody or an antigen-binding fragment thereof comprising:
(a) a heavy chain variable region (VH) comprising the amino acid sequence of SEQ ID NO: 1 (H-CDR1), the amino acid sequence of SEQ ID NO: 3 (H-CDR2), and the amino acid sequence of SEQ ID NO: 7 (H-CDR3); and
a light chain variable region (VL) comprising the amino acid sequence of SEQ ID NO: 9 (L-CDR1), the amino acid sequence of SEQ ID NO: 10 (L-CDR2), and the amino acid sequence of SEQ ID NO: 13 (L-CDR3); or
(b) a heavy chain variable region comprising the amino acid sequence of SEQ ID NO: 14, and a light chain variable region comprising the amino acid sequence of SEQ ID NO: 19; or
(c) a heavy chain comprising the amino acid sequence of SEQ ID NO: 26, and a light chain comprising the amino acid sequence of SEQ ID NO: 31.

98. The anti-IL-11 antibody or antigen-binding fragment thereof according to claim 87, wherein the antibody or antigen-binding fragment thereof:
(a) is a monoclonal antibody;
(b) is humanized; and/or
(c) binds to human IL-11 at a KD≤0.50 nM.

99. A pharmaceutical composition comprising the anti-IL-11 antibody or antigen-binding fragment thereof according to claim 87 and a pharmaceutically acceptable carrier and optionally comprising one or more additional therapeutic agents.

100. A method of treating idiopathic pulmonary fibrosis (IPF) or progressive pulmonary fibrosis (PPF) in a subject in need thereof, the method comprising:
administering to the subject a therapeutically effective amount of the anti-IL-11 antibody or antigen-binding fragment thereof according to claim 87 or a pharmaceutical composition comprising said antibody or antigen-binding fragment thereof, wherein optionally said antibody or antigen-binding fragment thereof is administered intravenously.

101. A method of treating metabolic dysfunction-associated steatohepatitis (MASH) in a subject in need thereof, the method comprising:
administering to the subject a therapeutically effective amount of the anti-IL-11 antibody or antigen-binding fragment thereof according to claim 87 or a pharmaceutical composition comprising said antibody or antigen-binding fragment thereof, wherein optionally said antibody or antigen-binding fragment thereof is administered intravenously.

102. A method of treating and/or preventing an IL-11 pathway disease or disorder in a subject in need thereof, the method comprising: administering to the subject a therapeutically effective amount of the anti-IL-11 antibody or antigen-binding fragment thereof according to claim 87 or a pharmaceutical composition comprising said antibody or antigen-binding fragment thereof.

103. The method according to claim 102, wherein the IL-11 pathway disease or disorder is:
(i) an inflammatory disease, autoimmune disease, respiratory disease, infectious disease, pulmonary disease, or fibrosis;
(ii) a liver disease or disorder, a lung disease or disorder, a musculoskeletal disease or disorder, a cardiac disease or disorder, a kidney disease or disorder, a metabolic disease or disorder, a cancer, a CNS disease or disorder, a lymphatic disease or disorder, an eye-related disease or disorder, an inflammatory disease or disorder, an autoimmune disease or disorder, or an infectious disease; and/or
(iii) SLD, MASH, early stage MASH, late stage MASH, other steatotic liver disease conditions (e.g., MASLD, MetALD, ALD, AH, specific etiology SLD, DILI, cryptogenic SLD, monogenic forms of SLD, Chronic liver disease, PBC, PSC, liver cirrhosis, compensated liver cirrhosis, decompensated liver cirrhosis alcohol-induced cirrhosis, steatohepatitis, alcoholic steatohepatitis, steatosis, ARLD, AFL, alcoholic hepatitis, ASH, alcohol-induced liver fibrosis (including early stage alcoholic liver fibrosis and later-stage fibrosis), hepatotoxicity/toxic hepatitis (acute or chronic), chronic active hepatitis, DILI (e.g., intrinsic or idiosyncratic hepatotoxicity, including allergic and nonallergic reaction), APAP-induced hepatotoxicity, ALI, acute liver failure, acute-on-chronic liver failure, acute liver disease, liver damage, hepatitis, viral hepatitis, alcoholic hepatitis, liver IRI, WIR, IDILI, autoimmune liver injury, cholestatic liver disease, HIV-associated liver injury, Wilson's Disease, Haemochromatosis), schistosomal liver disease, radiation-induced liver disease (RILD), iatrogenic causes (e.g., surgical damage to liver tissue), fasting, malnutrition, infection by infectious agents (e.g., hepatitis virus, HIV), cancer or drug interactions, sepsis, herbal or dietary supplements), pulmonary artery hypertension (PAH), COPD (chronic obstructive pulmonary disease), acute respiratory distress syndrome (ARDS), leiomyoma, leiomyosarcoma, progressive massive fibrosis, obliterative bronchiolitis, asbestosis, silicosis, AIDS-associated pulmonary hypertension, sarcoidosis, tumor stroma in lung disease, Hutchinson-Gilford Progeria Syndrome (HGPS), Hermansky-Pudlak Syndrome (HPS), cystic fibrosis, asthma, lung disease, AIDS associated pulmonary hypertension, sarcopenia, muscular dystrophy such as Duchenne muscular dystrophy (DMD), hip replacement failure, Becker's muscular dystrophy (BMD), myopenia, anorexic disorders (protein-energy malnutrition), lipodystrophies (e.g. abnormal or degenerative condition of adipose tissue), cardiomyopathy, cardiac or myocardial fibrosis (e.g., fibrosis in the heart associated with dysfunction of the musculature leading to CHF), hypertrophic cardiomyopathy (HCM), fibrosis of the atrium, fibrosis of the ventricle, myocardial fibrosis, endomyocardial fibrosis, myocardial infarction, fibrotic vascular disease, hypertensive heart disease, tubulointerstitial and glomerular fibrosis, atherosclerosis, cerebral infarcts, hypertension, vascular aneurysm, aortic aneurysm, familial thoracic aortic aneurysm syndrome, cerebral aneurysm, vascular stenosis and restenosis, renal artery stenosis, atrial fibrillation, Marfan's syndrome, Furlong's syndrome, Sphrintzen-Goldberg syndrome, Loeys-Dietz syndrome, arterial tortuosity syndrome, plexiform lesions, fibromuscular dysplasia (FMD), supravalvular stenosis, telangiectasia, varicose veins, Brugada syndrome, arrhythmogenic right ventricular cardiomyopathy (ARVC), dilated cardiomyopathy (DCM), ventricular fibrillation, myocarditis, kidney disease/renal disease (e.g., chronic kidney disease, renal fibrosis, nephritic syndrome, Alport's syndrome, HIV associated nephropathy, polycystic kidney disease, Fabry's disease, diabetic nephropathy, chronic glomerulonephritis, lupus nephritis/nephritis associated with systemic lupus, or kidney interstitial fibrosis (IF)), focal and segmental glomerulosclerosis (FSGS), IgA nephropathy, crescentic glomerulonephritis, kidney injury, acute kidney injury/renal failure, acute kidney failure, acute kidney disease, chronic kidney disease, kidney damage, autoimmune kidney injury, membranous nephropathy, nephrotoxicity, bladder disease, obesity, type 2 diabetes (T2D), type 1 diabetes (TID)/type 1 diabetes mellitus, pre-diabetes, being overweight, metabolic syndrome, gestational diabetes, insulin deficiency, pancreas injury, pregnancy-associated hyperglycemia, cholestasis (i.e., a reduced flow of bile from the liver to the duodenum), cholestatic liver disease (e.g., primary biliary cholangitis (PBC)), hyperglycemia, hyperlipidaemia, hypertriglyceridemia, hypercholesterolemia, wasting, “mild muscle wasting disease,” cachexia, pre-cachexia, refractory cachexia, sarcopenia, muscular dystrophies, steatosis (macrovesicular or microvesicular), lipotoxicity (including cells of the liver, kidney, heart and/or skeletal muscle), fatty liver disease, metabolic dysfunction-associated steatotic liver disease (MASLD), metabolic dysfunction-associated steatotic liver (MASL), insulin deficiency, insulin resistance, lipodystrophy, lipohypertrophy, lipoatrophy, chemotherapy-associated weight loss, pancreatic insufficiency, acute pancreatitis, chronic pancreatitis, hyperglucagonemia, Glioblastoma, breast cancer, colon cancer, gastric cancer, Leukemia, NSCLC, prostate cancer, hepatocellular carcinoma, epithelial cell cancer, gastrointestinal cancer (including oesophageal cancer, stomach cancer, pancreatic cancer, liver cancer (HCC), gallbladder cancer, colorectal cancer, anal cancer, gastrointestinal carcinoid tumour), lung cancer (including non-small cell lung cancer (NSCLC) and small cell lung cancer (SCLC)), Crohn's disease, ulcerative colitis, achalasia, dysphagia, diarrhoea, constipation, inflammatory bowel disease (IBD), bowel stricture, pyloric stenosis, coeliac disease, irritable bowel syndrome (IBS, irritable bowel syndrome), diverticulitis, microscopic colitis, primary sclerosing cholangitis (PSC), systemic sclerosis/scleroderma, progressive systemic sclerosis (PSS), chronic graft versus host disease, nephrogenic systemic fibrosis, cutis keloid, arthrofibrosis, Dupuytren's contracture, mediastinal fibrosis, retroperitoneal fibrosis, Peyronie's disease, adhesive capsulitis, fibrotic pre-neoplastic and fibrotic neoplastic disease, fibrosis induced by chemical or environmental insult (e.g., cancer chemotherapy, pesticides, radiation/cancer radiotherapy), arthritis, multiple sclerosis, gliosis, Alzheimer's disease, myelofibrosis, myeloproliferative diseases, aplastic anemia, thyroid eye disease, retinopathy, diabetic retinopathy, chronic pulmonary hypertension, Grave's ophthalmopathy, epiretinal fibrosis (e.g. diabetic retinopathy (DR)), glaucoma, subretinal fibrosis (e.g. associated with macular degeneration (e.g. wet or dry age-related macular degeneration (AMD))), macular edema, drusen formation, choroidal neovascularization (CNV), post-surgical fibrosis (e.g. of the posterior capsule following cataract surgery, or of the bleb following trabeculectomy for glaucoma), conjunctival fibrosis, subconjunctival fibrosis, retinal fibrosis (e.g. associated with wet age-related macular degeneration (AMD)), an Interstitial lung disease (ILD), including idiopathic pulmonary fibrosis (IPF), progressive pulmonary fibrosis (PPF), fibrotic ILA, scleroderma affecting the lung, or scleroderma-associated ILD, an inflammatory disease or disorder, an autoimmune disease or disorder, or an infectious disease.

104. The method according to claim 102, wherein the IL-11 pathway disease or disorder is MASH, early stage MASH, late stage MASH, sarcopenia, cardiomyopathy, cardiac or myocardial fibrosis, kidney disease/renal disease (e.g., chronic kidney disease, renal fibrosis, nephritic syndrome, Alport's syndrome, HIV associated nephropathy, polycystic kidney disease, Fabry's disease, diabetic nephropathy, chronic glomerulonephritis, lupus nephritis/nephritis associated with systemic lupus, or kidney IF), sarcopenia, ILD, IPF, PPF, fibrotic ILA, scleroderma affecting the lung, or scleroderma-associated ILD.

105. A polynucleotide or plurality of polynucleotides encoding the heavy chain variable region and/or the light chain variable region according to claim 87.

106. A vector or plurality of vectors or a host cell or plurality of host cells or a mammalian host cell or plurality of mammalian host cells, wherein said vector(s), host cell(s), or mammalian host cell(s) comprise the polynucleotide or plurality of polynucleotides according to claim 105.

107. A polynucleotide or plurality of polynucleotides encoding the heavy chain and/or the light chain according to claim 95.

108. A vector or plurality of vectors or a host cell or plurality of host cells or a mammalian host cell or plurality of mammalian host cells, wherein said vector(s), host cell(s), or mammalian host cell(s) comprise the polynucleotide or plurality of polynucleotides according to claim 107.

109. A method for the production of an anti-IL-11 antibody or antigen-binding fragment thereof, comprising the steps:
(a) cultivating the host cell(s) or mammalian host cell(s) according to claim 106 under conditions suitable for expression of the anti-IL-11 antibody or antigen-binding fragment thereof; and
(b) recovering the anti-IL-11 antibody or antigen-binding fragment thereof; and optionally:
(i) further comprising the step: (c) purifying the anti-IL-11 antibody or antigen-binding fragment thereof or
(ii) further comprising the steps: (c) purifying the anti-IL-11 antibody or antigen-binding fragment thereof and (d) formulating the anti-IL-11 antibody or antigen-binding fragment thereof into a pharmaceutical composition.

110. A diagnostic kit or diagnostic method comprising the anti-human IL-11 antibody or antigen-binding fragment thereof according to claim 87.

111. An anti-human IL-11 antibody or antigen-binding fragment thereof, wherein when bound to human IL-11, the antibody or antigen-binding fragment thereof binds to amino acid residues R15, D19, L23, R26, S27 L29, A30, D31, R33, R40, N50, L57, A58, R111, H161, L162, D165, W166, V168, R169, L172, L173, K175, or T176 as set forth in SEQ ID NO: 49, and wherein the antibody blocks binding of IL-11 to IL-11Rα.
